(19) United States
(12) Patent Application Publication
(10) Pub. No.: US 2012/0165334 A1
(43) Pub. Date: Jun. 28, 2012

(54) Indole/Benzimidazole Compounds as mTOR Kinase Inhibitors

(75) Inventors: Alessandro Boezio, Somerville, MA (US); Alan C. Cheng, San Francisco, CA (US); James Robert Coats, Oak Harbor, WA (US); Katrina Woodin Copeland, Sudbury, MA (US); Russell Graceffa, Hampton, NH (US); Jean-Christophe Harmanage, Andover, MA (US); Hongbing Huang, Lexington, MA (US); Daniel La, Brookline, MA (US); Philip R. Olivieri, Charleston, MA (US); Emily Anne Peterson, Garobridge, MA (US); Laurie Schenkel, Boston, MA (US)

(73) Assignee: AMGEN INC., Thousand Oaks, CA (US)

(21) Appl. No.: 13/260,715

(22) PCT Filed: Feb. 10, 2010
(86) PCT No.: PCT/US2010/023764

§ 371 Date: Mar. 7, 2012

Related U.S. Application Data
(60) Provisional application No. 61/153,580, filed on Feb. 18, 2009.

(51) Int. Cl. A61K 31/53; A61P 35/00; C07D 403/04; A61K 31/506; C07D 401/14; C07D 403/14

(52) U.S. Cl. 514/245; 544/209; 544/328; 514/256; 544/324; 514/275

(57) ABSTRACT

The present invention provides compounds that are kinase inhibitors, specifically PIK kinase inhibitors, more specifically, mTOR inhibitors and are therefore useful for the treatment of diseases treatable by inhibition of kinases, specifically PIK kinase inhibitors, more specifically, mTOR such as cancer. Also provided are pharmaceutical compositions containing such compounds and processes for preparing such compounds.

### CROSS-REFERENCE

This application claims the benefit of U.S. Provisional Application No. 61/153,580, filed Feb. 18, 2009, the disclosure of which is incorporated herein by reference in its entirety.

### FIELD OF THE INVENTION

The present invention provides compounds that are kinase inhibitors, specifically PIK kinase inhibitors, more specifically, mTOR inhibitors and are therefore useful for the treatment of diseases treatable by inhibition of kinases, specifically PIK kinase inhibitors, more specifically, mTOR such as cancer. Also provided are pharmaceutical compositions containing such compounds and processes for preparing such compounds.

### BACKGROUND

Mammalian target of rapamycin (mTOR) is a serine/threonine kinase of approximately 289 kDa in size and a member of the evolutionary conserved eukaryotic TOR kinases. The mTOR protein is a member of the PI3-kinase like kinase (PIKK) family of proteins due to its C-terminal homology (catalytic domain) with PI3-kinase and the other family members, e.g. DNA dependent protein kinase (DNA-PKcs), Ataxia-telangiectasia mutated (ATM).

It has been demonstrated that mTOR kinase is a central regulator of cell growth and survival by mediating multiple important cellular functions including translation, cell cycle regulation, cytoskeleton reorganization, apoptosis and autophagy. mTOR resides in two biochemically and functionally distinct complexes that are conserved from yeast to human. The rapamycin sensitive mTOR-Raptor complex (mTORC1) regulates translation by activation of p70S6 kinase and inhibition of eIF4E binding protein 4EBP1 through phosphorylation, which is the best-described physiological function of mTOR signaling. mTORC1 activity is regulated by extracellular signals (growth factors and hormones) through the PI3K/AKT pathway, and by nutrient availability, intracellular energy status and oxygen through the regulators like LKB1 and AMPK. Rapamycin and its analogues inhibit mTORC1 activity by disrupting the interaction between mTOR and Raptor. The rapamycin-insensitive complex, mTORC2, was discovered only recently. Unlike mTORC1 which contains raptor, the mTORC2 complex contains other proteins including Rictor and mSin1. mTORC2 phosphorylates AKT at the hydrophobic Ser473 site, and appears to be essential for AKT activity. Other substrates of mTORC2 include PKCα and SGK1. How mTORC2 activity is regulated is not well understood.

The mTORC1 pathway can be activated by elevated PI3K/AKT signaling or mutations in the tumor suppressor genes PTEN or TSC2, providing cells with a growth advantage by promoting protein synthesis. Cancer cells treated with the mTORC1 inhibitor rapamycin show growth inhibition and, in some cases, apoptosis. Three rapamycin analogues, CCI-779 (Wyeth), RAD001 (Novartis) and AP23573 (Ariad) are in clinical trials for the treatment of cancer. However response rates vary among cancer types from a low of less than 10% in patients with glioblastoma and breast cancer to a high of around 40% in patients with mantle cell lymphoma. Recent studies demonstrated that rapamycin can actually induce a strong AKT phosphorylation in tumors by attenuating the feedback inhibition on receptor tyrosine kinases mediated by p70S6K, one of the downstream effectors of mTORC1. For example, in Phase I clinical trials of RAD001, an increase in pAKT (+22.2 to 63.1% of initial values) was observed after dosing. If mTORC1 inhibition-induced phospho-AKT leads to increased cancer cell survival and acquisition of additional lesions, this could counteract the effects of growth inhibition by rapamycin analogues and explain the variable response rate. Therefore, identifying and developing small molecules that target the catalytic activity of mTOR (inhibiting both mTORC1 and mTORC2) may lead to more effective therapeutics to treat cancer patients by preventing the activation of AKT that is caused by mTORC1 specific inhibitors like rapamycin and its analogues. Dysregulated mTOR activity has been shown to associate with variety of human cancers such as breast, lung, kidney, brain, ovarian, colon, cervical, endometrial, prostate, liver, thyroid, GI tract, blood and lymphoma and other diseases such as hamartoma syndromes, rheumatoid arthritis, multiple sclerosis. In view of the important role of mTOR in biological processes and disease states, catalytic inhibitors of this protein kinase are desirable. The present invention provides kinase inhibitors, specifically PIK kinase inhibitors, more specifically, mTOR inhibitors, which are useful for treating diseases mediated by kinases, specifically PIK kinases, more specifically, mTOR.

### SUMMARY

In one aspect, provided herein are compounds of Formula (I):

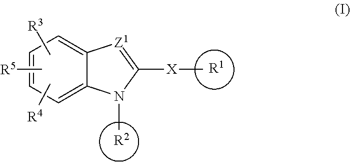

where:

Z¹ is —N— or —CH—;

X is —NR⁶— or —O— where R⁶ is hydrogen or alkyl;

R¹ is aryl, heteroaryl, cycloalkyl, fused cycloalkenyl, or heterocyclyl; each ring substituted with R^{a}, R^{b}, or R^{c} independently selected from hydrogen, alkyl, alkylthio, alkoxy, hydroxy, alkoxycarbonyl, carboxy, halo, haloalkyl, haloalkoxy, aminocarbonyl, aminosulfonyl, cycloalkyl, cycloalkylalkyl, acyl, cyano, aminoalkyl, hydroxyalkyl, optionally substituted heteroaryl, optionally substituted phenyl, amino, ureido, thioureido, monosubstituted, or disubstituted amino;

R² is:

(i)

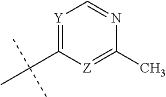

where Y and Z are independently —N═ or —C═; or

(ii) a five or six membered heterocyclyl ring;

each ring in (i) and (ii) is substituted with R^{d} and R^{e} where R^{d} and R^{e} are independently hydrogen, alkyl, alkoxy, halo, haloalkyl, haloalkoxy, amino, monosubstituted amino or disubstituted amino;

R³ and R⁴ are independently hydrogen, alkyl, halo, alkoxy, haloalkyl, hydroxyalkyl, alkoxyalkyl, cyano, carboxy, alkoxycarbonyl, aryl, heteroaryl, heterocyclyl, aralkyl, heteroaralkyl, heterocyclylalkyl, amino, monosubstituted amino, disubstituted amino, sulfonyl, acyl, hydroxyalkyloxy, alkoxyalkyloxy, aminoalkyl, aminoalkoxy, aryloxy, heteroaryloxy, heterocyclyloxy, aralkoxy, heteroaralkoxy, heterocyclylalkyloxy, aminosulfonyl, aminocarbonyl, or acylamino, where the aromatic or alicyclic ring in R³ and R⁴ is optionally substituted with R^{f}, R^{g} or R^{h} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; and

R⁵ is hydrogen, alkyl, halo, hydroxyl, alkoxy, haloalkyl, hydroxyalkyl, alkoxyalkyl, cyano, carboxy, alkoxycarbonyl, amino, alkylamino, or dialkylamino; or

a pharmaceutically acceptable salt thereof; provided that:

(i) when Z¹ is —N═, R² is piperidin-4-yl, 4-methylpiperidin-1-yl, or 1-methylpiperidin-4-yl; X is —NH—, R³ is hydrogen, and R¹ is phenyl substituted at the 4-position with ethyl or —COR where R is methylamino, methoxy, methyl, or amino; 3,4,5-trimethyloxyphenyl, or 3,5-dimethoxyphenyl, then R⁴ is not —CON(CH₂CH₂CH(CH₃)₂)₂; or —CON(i-Bu)₂;

(ii) when Z¹ is —N═, R² is 6-chloro-5-methylpyrimidin-4-yl, 5-methyl-6-[4-diethylaminobutylamino]-pyrimidin-4-yl, or 6-amino-5-methylpyrimidin-4-yl, X is —NH—, R³ and R⁴ are hydrogen, then R¹ is not 6-methyl-3-(3-trifluoromethylphenyl)carbonylamino)-phenyl;

(iii) when Z¹ is —N═, R² is tetrahydropyran-2-yl, X is —NH—, R³ and R⁴ are hydrogen, then R¹ is not piperidin-4-yl or 1-ethoxycarbonylpiperidin-4-yl; and

(iv) the compound is not 1-(4-amino-6-methyl-1,3,5-triazin-2-yl)-N-3-pyrrolidinyl-1H-benzimidazole-2-amine and 1-(4-amino-6-methyl-1,3,5-triazin-2-yl)-N-1H-imidazol-2-yl-1H-benzimidazole-2-amine

In another aspect, the compound of Formula (I) is where R³ and R⁴ is substituted with R^{f}, R^{g} or R^{h} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; and

R⁵ is hydrogen, alkyl, halo, alkoxy, haloalkyl, hydroxyalkyl, alkoxyalkyl, cyano, carboxy, alkoxycarbonyl, amino, alkylamino, or dialkylamino

In a second aspect, provided is a pharmaceutical composition comprising a compound of Formula (I), a pharmaceutically acceptable salt thereof, or a mixture of a compound of Formula (I) and a pharmaceutically acceptable salt thereof; and a pharmaceutically acceptable excipient.

In a third aspect, this invention is directed to a method of treatment of a disease mediated by kinases, specifically PIK kinases, more specifically mTOR, in a patient which method comprises administering to the patient a pharmaceutical composition comprising a compound of Formula (I) or a pharmaceutically acceptable salt thereof, or a mixture of a compound of Formula (I) and a pharmaceutically acceptable salt thereof and a pharmaceutically acceptable excipient. In one embodiment the disease is human cancers such as breast, lung, kidney, brain, ovarian, colon, cervical, endometrial, prostate, liver, thyroid, GI tract, blood and lymphoma and other diseases such as hamartoma syndromes, rheumatoid arthritis, and multiple sclerosis.

In a fourth aspect, this invention is directed to use of a compound of Formula (I) in the manufacture of a medicament for the treatment of a disease mediated by kinases, specifically PIK kinases, more specifically mTOR, even more specifically for the treatment of cancers, more specifically in the treatment of cancers such as breast, lung, kidney, brain, ovarian, colon, cervical, endometrial, prostate, liver, thyroid, GI tract, blood and lymphoma and other diseases such as hamartoma syndromes, rheumatoid arthritis, and multiple sclerosis.

In a fifth aspect, this invention is directed to compounds of Formula (I) for use in therapy, preferably the therapy is treatment of cancers, more specifically in the treatment of cancers such as breast, lung, kidney, brain, ovarian, colon, cervical, endometrial, prostate, liver, thyroid, GI tract, blood and lymphoma and other diseases such as hamartoma syndromes, rheumatoid arthritis, and multiple sclerosis provided that:

(i) when Z¹ is —N═, R² is piperidin-4-yl, 4-methylpiperidin-1-yl, or 1-methylpiperidin-4-yl; X is —NH—, R³ is hydrogen, and R¹ is phenyl substituted at the 4-position with ethyl or —COR where R is methylamino, methoxy, methyl, or amino; 3,4,5-trimethyloxyphenyl, or 3,5-dimethoxyphenyl, then R⁴ is not —CON(CH₂CH₂CH(CH₃)₂)₂; or —CON(i-Bu)₂,

(ii) when Z¹ is —N═, R² is 6-chloro-5-methylpyrimidin-4-yl, 5-methyl-6-[4-diethylaminobutylamino]-pyrimidin-4-yl, or 6-amino-5-methylpyrimidin-4-yl, X is —NH—, R³ and R⁴ are hydrogen, then R¹ is not 6-methyl-3-(3-trifluoromethylphenylcarbonylamino)-phenyl; and

(iii) when Z¹ is —N═, R² is tetrahydropyran-2-yl, X is —NH—, R³ and R⁴ are hydrogen, then R¹ is not piperidin-4-yl or 1-ethoxycarbonylpiperidin-4-yl

(iii) when Z¹ is —N═, R² is tetrahydropyran-2-yl, X is —NH—, R³ and R⁴ are hydrogen, then R¹ is not piperidin-4-yl or 1-ethoxycarbonylpiperidin-4-yl; and

(iv) the compound if not 1-(4-amino-6-methyl-1,3,5-triazin-2-yl)-N-3-pyrrolidinyl-1H-benzimidazole-2-amine and 1-(4-amino-6-methyl-1,3,5-triazin-2-yl)-N-1H-imidazol-2-yl-1H-benzimidazole-2-amine

### DETAILED DESCRIPTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring e.g., cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means an —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with one or two alkoxy groups, as defined above, e.g., 2-methoxyethyl, 1-, 2-, or 3-methoxypropyl, 2-ethoxyethyl, and the like.

“Alkoxyalkyloxy” or “alkoxyalkoxy” means an —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with one or two, —NRR′ where R is hydrogen, alkyl, or —COR″ where R″ is alkyl, each as defined above, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical where R is aminoalkyl as defined above, e.g., 2-aminoethoxy, 2-dimethylaminopropoxy, and the like.

“Aminocarbonyl” means a —CONRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, aminoalkyl, substituted aryl, or substituted heteroaryl, each as defined herein. Preferably, R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, e.g., —CONH₂, methylaminocarbonyl, dimethylaminocarbonyl, and the like.

“Aminosulfonyl” means a —SO₂NRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, aminoalkyl, substituted aryl or substituted heteroaryl, each as defined herein. Preferably, R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, e.g., —SO₂NH₂, methylaminosulfonyl, dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, heterocyclylalkyl, substituted aryl or substituted heteroaryl, each as defined herein. Preferably R is alkyl, haloalkyl, cycloalkyl, cycloalkylalkyl, aryl, aralkyl, heteroaryl, heteroaralkyl, heterocyclyl, or heterocyclylalkyl, e.g., acetyl, propionyl, benzoyl, pyridinylcarbonyl, and the like. When R is alkyl, the radical is also referred to herein as alkylcarbonyl.

“Acylamino” means an —NHCOR radical where R is alkyl, haloalkyl, cycloalkyl, cycloalkylalkyl, amino, monosubstituted or disubstituted amino, aryl, aralkyl, heteroaryl, heteroaralkyl, heterocyclyl, heterocyclylalkyl, substituted aryl or substituted heteroaryl, each as defined herein. Preferably R is alkyl, haloalkyl, cycloalkyl, cycloalkylalkyl, amino, monosubstituted or disubstituted amino, aryl, aralkyl, heteroaryl, heteroaralkyl, heterocyclyl, or heterocyclylalkyl, e.g., acetylamino, propionylamino, and the like.

“Aryl” means a monovalent monocyclic or bicyclic aromatic hydrocarbon radical of 6 to 10 ring atoms e.g., phenyl or naphthyl.

“Aralkyl” means an -(alkylene)-R radical where R is aryl as defined above.

“Aryloxy” means an —OR radical where R is aryl as defined above, e.g., phenoxy, naphthyloxy.

“Aralkyloxy” means an —OR radical where R is aralkyl as defined above, e.g., benzyloxy, and the like.

“Cyanoalkyl” means an -(alkylene)-CN radical e.g., cyanomethyl, and the like.

“Cycloalkyl” means a cyclic saturated monovalent hydrocarbon radical of three to ten carbon atoms, e.g., cyclopropyl, cyclobutyl, cyclopentyl, or cyclohexyl, and the like.

“Cycloalkylalkyl” means an -(alkylene)-R radical where R is cycloalkyl as defined above; e.g., cyclopropylmethyl, cyclobutylmethyl, cyclopentylethyl, or cyclohexylmethyl, and the like.

“Carboxy” means —COOH.

“Disubstituted amino” means a —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, aminoalkyl, substituted aryl or substituted heteroaryl, each as defined herein. Preferably R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, e.g., dimethylamino, phenylmethylamino, and the like. When R and R′ are alkyl, it is also referred to herein as dialkylamino

“Fused cycloalkenyl” means an unsaturated cyclic monovalent hydrocarbon radical of three to ten carbon atoms that is fused to phenyl and wherein one or two of the carbon atoms are replaced by a —C═O group, e.g., indenyl, 1-oxo-2,3-dihydroindenyl, and the like.

“Halo” means fluoro, chloro, bromo, or iodo, preferably fluoro or chloro.

“Haloalkyl” means alkyl radical as defined above, which is substituted with one or more halogen atoms, preferably one to five halogen atoms, preferably fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it is referred to in this Application as fluoroalkyl.

“Haloalkoxy” means an —OR radical where R is haloalkyl as defined above e.g., —OCF₃, —OCHF₂, and the like. When R is haloalkyl where the alkyl is substituted with only fluoro, it is referred to in this Application as fluoroalkoxy.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means an —OR radical where R is hydroxyalkyl as defined above.

“Heterocyclyl” means a saturated or unsaturated monovalent monocyclic group of 4 to 8, preferably 5 to 8, ring atoms in which one or two ring atoms are heteroatom selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring and is a subset of fused heterocyclyl. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

“Heterocyclylalkyl” means an -(alkylene)-R radical where R is heterocyclyl ring as defined above e.g., tetraydrofuranylmethyl, piperazinylmethyl, morpholinylethyl, and the like.

“Heterocyclyloxy” means an —OR radical where R is heteroacyclyl as defined above, e.g., piperidinyloxy, and the like.

“Heterocyclylalkyloxy” means an —O-(alkylene)-R radical where R is heterocyclyl ring as defined above e.g., tetraydrofuranylmethyloxy, piperazinylmethyloxy, morpholinylethyloxy, and the like.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like.

“Heteroaralkyl” means an -(alkylene)-R radical where R is heteroaryl as defined above.

“Heteraryloxy” means an —OR radical where R is heteroaryl as defined above, e.g., pyridinyloxy, thiophenyloxy, and the like.

“Heteroaralkyloxy” means an —O-(alkylene)-R radical where R is heteroaryl as defined above.

“Monosubstituted amino” means a —NHR radical where R is alkyl, cyanoalkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, aminoalkyl, substituted aryl or substituted heteroaryl, each as defined herein. Preferably, R is alkyl, cyanoalkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, e.g., methylamino, phenylamino, hydroxyethylamino, and the like.

The present invention also includes the prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I).

For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

The present invention also includes deuterium analogs of compounds of Formula (I).

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Oxo” or “carbonyl” means —C═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

“Optional substituted phenyl” means phenyl ring that is optionally substituted with one, two, or three substitutents independently selected from alkyl, halo, haloalkyl, hydroxyl, haloalkoxy, alkoxy, alkoxycarbonyl, amino, alkylamino, cyano, or dialkylamino, preferably alkyl, halo, haloalkyl, hydroxyl, haloalkoxy, alkoxy, amino, alkylamino, cyano, or dialkylamino

“Optional substituted heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon, that is optionally substituted with one, two, or three substitutents independently selected from alkyl, halo, haloalkyl, haloalkoxy, alkoxy, alkoxycarbonyl, amino, alkylamino, cyano, or dialkylamino. Preferably, alkyl, halo, haloalkyl, haloalkoxy, alkoxy, amino, alkylamino, cyano, or dialkylamino

“Optional substituted heterocyclyl” means heterocyclyl as defined above, that is optionally substituted with one, two, or three substitutents independently selected from alkyl, halo, haloalkyl, haloalkoxy, alkoxy, alkoxycarbonyl, amino, alkylamino, cyano, or dialkylamino

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfonyl” means a —SO₂R radical where R is alkyl, haloalkyl, aryl, aralkyl, heteroaryl, heteroaralkyl, heterocyclyl, heterocyclylalkyl, substituted aryl or substituted heteroaryl, each as defined herein. Preferably, R is alkyl, haloalkyl, aryl, aralkyl, heteroaryl, heteroaralkyl, heterocyclyl, or heterocyclylalkyl, e.g., methylsulfonyl, phenylsulfonyl, benzylsulfonyl, pyridinylsulfonyl, and the like.

“Substituted aryl” means aryl ring as defined above that is substituted with one, two, or three substitutents independently selected from alkyl, halo, haloalkyl, haloalkoxy, alkoxy, alkoxycarbonyl, amino, alkylamino, cyano, or dialkylamino

“Substituted heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon, that is substituted with one, two, or three substitutents independently selected from alkyl, halo, haloalkyl, haloalkoxy, alkoxy, alkoxycarbonyl, amino, alkylamino, cyano, or dialkylamino

The phrase in the definition of groups Ar¹ and Ar² in the claims and in the specification of this Application “ . . . wherein the aforementioned rings are optionally substituted with R^{a}, R^{b}, or R^{c} independently selected from” and similar phrases used for others groups in the claims and in the specification with respect to the compound of Formula (I) means that the rings can be mono-, di-, or trisubstituted unless indicated otherwise.

“Treating” or “treatment” of a disease includes:

preventing the disease, i.e. causing the clinical symptoms of the disease not to develop in a mammal that may be exposed to or predisposed to the disease but does not yet experience or display symptoms of the disease;

inhibiting the disease, i.e., arresting or reducing the development of the disease or its clinical symptoms; or

relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

“Thioureido” means a —NHCSNHR radical where R is hydrogen, alkyl, optionally substituted phenyl, or optionally substituted heteroaryl as defined above e.g., 3-methylureido, 3-ethylureido, and the like.

“Ureido” means a —NHCONHR radical where R is hydrogen, alkyl, optionally substituted phenyl, or optionally substituted heteroaryl as defined above e.g., 3-methylureido, 3-ethylureido, and the like.

Representative compounds of the Invention where X¹ is nitrogen and R⁵ is hydrogen, and other groups are as shown in shown in Table 1 below:

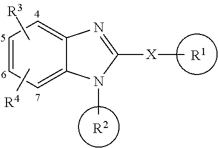

| | | | | | | | Mass |
|---|---|---|---|---|---|---|---|
| Cpd | | | | | | Mass | obs. |
| # | X | R¹ | R² | R³ | R⁴ | cal. | (M + H) |
| 1 | —NH— | 3,5- | 4-amino-6-methyl- | H | H | 349.3 | 350.4 |
| | | dihydroxyphenyl | 1,3,5-triazin-2-yl | | | | |
| 2 | —NH— | 3-hydroxyphenyl | 4-amino-6-methyl- | H | 6- | 377.4 | 378 |
| | | | 1,3,5-triazin-2-yl | | CO₂H | | |
| 3 | —NH— | 3-hydroxyphenyl | 4-amino-6-methyl- | H | H | 333.4 | 334.0 |
| | | | 1,3,5-triazin-2-yl | | | | |
| 4 | —NH— | 3-(—NHCONHCH₃)- | 4-amino-6-methyl- | H | H | 389.4 | 390.2 |
| | | phenyl | 1,3,5-triazin-2-yl | | | | |
| 5 | —NH— | 3-(—NHCONHCH₂CH₃)- | 4-amino-6-methyl- | H | H | 403.4 | 404.3 |
| | | phenyl | 1,3,5-triazin-2-yl | | | | |
| 6 | —NH— | 3-hydroxyphenyl | 4-cyanomethylamino- | H | H | 372.4 | 373 |
| | | | 6-methyl-1,3,5-triazin- | | | | |
| | | | 2-yl | | | | |
| 7 | —NH— | 3-(3-hydroxyphenyl- | 4-amino-6-methyl- | H | H | 467.5 | 468.2 |
| | | NHCONH—)phenyl | 1,3,5-triazin-2-yl | | | | |
| 8 | —NH— | 3-hydroxy-5- | 4-amino-6-methyl- | H | H | 391.4 | 392.3 |
| | | methoxycarbonyl- | 1,3,5-triazin-2-yl | | | | |
| | | phenyl | | | | | |
| 9 | —NH— | pyrazol-5-yl | 4-amino-6-methyl- | 5-F | 6-F | 343.3 | 344.2 |
| | | | 1,3,5-triazin-2-yl | | | | |
| 10 | —NH— | pyrazol-3-yl | 4-amino-6-methyl- | H | H | 307.3 | 308.4 |
| | | | 1,3,5-triazin-2-yl | | | | |
| 11 | —NH— | pyrazol-5-yl | 4-amino-6-methyl- | H | mixture | 325.3 | 326.4 |
| | | | 1,3,5-triazin-2-yl | | of 5 | | |
| | | | | | and 6-F | | |
| 12 | —NH— | 3-hydroxy-4- | 4-amino-6-methyl- | H | H | 363.4 | 364.2 |
| | | methoxy-phenyl | 1,3,5-triazin-2-yl | | | | |
| 13 | —NH— | 3,5- | 4-amino-6-ethyl-1,3,5- | H | H | 363.4 | 364.3 |
| | | dihydroxyphenyl | triazin-2-yl | | | | |
| 14 | —NH— | indazol-6-yl | 4-amino-6-methyl- | H | H | 357.4 | 358.2 |
| | | | 1,3,5-triazin-2-yl | | | | |
| 15 | —NH— | indol-6-yl | 4-amino-6-methyl- | H | H | 356.4 | 357.2 |
| | | | 1,3,5-triazin-2-yl | | | | |
| 16 | —NH— | 3-cyclopropyl- | 4-amino-6-methyl- | H | H | 347.4 | 348.3 |
| | | pyrazol-5-yl | 1,3,5-triazin-2-yl | | | | |
| 17 | —NH— | 3-(3- | 4-amino-6-methyl- | H | H | 481.5 | 482.2 |
| | | methoxyphenyl- | 1,3,5-triazin-2-yl | | | | |
| | | NHCONH—)phenyl | | | | | |
| 18 | —NH— | 3-(4- | 4-amino-6-methyl- | H | H | 509.5 | 510.2 |
| | | methoxycarbonylphenyl- | 1,3,5-triazin-2-yl | | | | |
| | | NHCONH—)phenyl | | | | | |
| 19 | —NH— | 3-hydroxyphenyl | 4-amino-6-methyl- | 5-CH₃ | 6-CH₃ | 361.4 | 362.4 |
| | | | 1,3,5-triazin-2-yl | | | | |
| 20 | —NH— | 2-aminopyridin-5-yl | 4-amino-6-methyl- | H | H | 333.4 | 334.2 |
| | | | 1,3,5-triazin-2-yl | | | | |
| 21 | —NH— | indazol-3-yl | 4-amino-6-methyl- | H | H | 357.4 | 358.2 |
| | | | 1,3,5-triazin-2-yl | | | | |
| 22 | —NH— | 4-carboxy-2- | 4-amino-6-methyl- | H | H | 377.4 | 378.3 |
| | | hydroxyphenyl | 1,3,5-triazin-2-yl | | | | |
| 23 | —NH— | indol-4-yl | 4-amino-6-methyl- | H | H | 356.4 | 357 |
| | | | 1,3,5-triazin-2-yl | | | | |
| 24 | —NH— | 4-fluoroindazol-3-yl | 4-amino-6-methyl- | H | H | 375.4 | 376.2 |
| | | | 1,3,5-triazin-2-yl | | | | |
| 25 | —NH— | 4-hydroxyphenyl | 4-amino-6-methyl- | H | H | 333.3 | 334 |
| | | | 1,3,5-triazin-2-yl | | | | |
| 26 | —NH— | 3-carboxyphenyl | 4-amino-6-methyl- | H | H | 361.4 | 362 |
| | | | 1,3,5-triazin-2-yl | | | | |
| 27 | —NH— | pyrazol-3-yl | 4-amino-6-methyl- | H | mixture | 386.2 | 386.1 |
| | | | 1,3,5-triazin-2-yl | | of 5 or | | |
| | | | | | 6-Br | | |
| 28 | —NH— | pyrazol-5-yl | 4-amino-6-methyl- | 5-CH₃ | 6-CH₃ | 335.4 | 336.3 |
| | | | 1,3,5-triazin-2-yl | | | | |
| 29 | —NH— | pyridin-3-yl | 4-amino-6-methyl- | H | H | 318.3 | 319.2 |
| | | | 1,3,5-triazin-2-yl | | | | |
| 30 | —NH— | isoxazol-3-yl | 4-amino-6-methyl- | H | H | 308.3 | 309.2 |
| | | | 1,3,5-triazin-2-yl | | | | |
| 31 | —NH— | pyrazol-4-yl | 4-amino-6-methyl- | H | H | 307.3 | 308.4 |
| | | | 1,3,5-triazin-2-yl | | | | |
| 32 | —NH— | 4-fluoro-3-hydroxy- | 4-amino-6-methyl- | H | H | 351.3 | 352 |
| | | phenyl | 1,3,5-triazin-2-yl | | | | |
| 33 | —NH— | 3-methoxyphenyl | 4-amino-6-methyl- | H | 5- | 405.4 | 406 |
| | | | 1,3,5-triazin-2-yl | | COOCH₃ | | |
| 34 | —NH— | 3-CONH₂phenyl | 4-amino-6-methyl- | H | H | 360.4 | 361.2 |
| | | | 1,3,5-triazin-2-yl | | | | |
| 35 | —NH— | 3-hydroxy-4- | 4-amino-6-methyl- | H | H | 347.4 | 348 |
| | | methyl-phenyl | 1,3,5-triazin-2-yl | | | | |
| 36 | —NH— | 3-methoxyphenyl | 4-amino-6-methyl- | H | 5- | 377.4 | 378 |
| | | | 1,3,5-triazin-2-yl | | CH₂OH | | |
| 37 | —NH— | 4-(4-fluorophenyl- | 4-amino-6-methyl- | H | H | 491.2 | 490.1 |
| | | sulfonyl)- | 1,3,5-triazin-2-yl | | | | |
| | | aminophenyl | | | | | |
| 38 | —NH— | 6-aminopyridin-2-yl | 4-amino-6-methyl- | H | H | 333.4 | 334.2 |
| | | | 1,3,5-triazin-2-yl | | | | |
| 39 | —NH— | 1-oxo-2,3- | 4-amino-6-methyl- | H | H | 371.4 | 372.2 |
| | | dihydroinden-6-yl | 1,3,5-triazin-2-yl | | | | |
| 40 | —NH— | 1-methylpyrazol-3- | 4-amino-6-methyl- | H | H | 321.3 | 322.2 |
| | | yl | 1,3,5-triazin-2-yl | | | | |
| 41 | —NH— | 3-methoxyphenyl | 4-amino-6-methyl- | H | H | 347.4 | 348 |
| | | | 1,3,5-triazin-2-yl | | | | |
| 42 | —NH— | 3-cyclopropyl- | 4-amino-6-methyl- | 5-CH₃ | 6-CH₃ | 375.4 | 376.2 |
| | | pyrazol-5-yl | 1,3,5-triazol-2-yl | | | | |
| 43 | —NH— | 4-(4-methylphenyl- | 4-amino-6-methyl- | H | H | 487.2 | 486.2 |
| | | sulfonyl)- | 1,3,5-triazin-2-yl | | | | |
| | | aminophenyl | | | | | |
| 44 | —NH— | 3-methylisothiazol- | 4-amino-6-methyl- | H | H | 338.4 | 339.2 |
| | | 5-yl | 1,3,5-triazin-2-yl | | | | |
| 45 | —NH— | 3-SO₂NH₂phenyl | 4-amino-6-methyl- | H | H | 396.4 | 397.2 |
| | | | 1,3,5-triazin-2-yl | | | | |
| 46 | —NH— | phenyl | 4-amino-6-methyl- | H | H | 317.3 | 318.3 |
| | | | 1,3,5-triazin-2-yl | | | | |
| 47 | —NH— | 4- | 4-amino-6-methyl- | H | H | 411.3 | 410.1 |
| | | methylsulfonylamino- | 1,3,5-triazin-2-yl | | | | |
| | | phenyl | | | | | |
| 48 | —NH— | 3-methoxyphenyl | 4-amino-6-methyl- | 6-CH₂OH | H | 377.4 | 378 |
| | | | 1,3,5-triazin-2-yl | | | | |
| 49 | —NH— | 3-hydroxy-4- | 4-amino-6-methyl- | H | H | 391.4 | 392.2 |
| | | methoxycarbonylphenyl | 1,3,5-triazin-2-yl | | | | |
| 50 | —NH— | 5-methylpyrazol-3-yl | 4-amino-6-methyl- | H | H | 321.3 | 322.2 |
| | | | 1,3,5-triazin-2-yl | | | | |
| 51 | —NH— | pyrazol-3-yl | 4-methylamino-6- | H | H | 321.3 | 322.2 |
| | | | methyl-1,3,5-triazin-2- | | | | |
| | | | yl | | | | |
| 52 | —NH— | 3-furan-2-ylpyrazol- | 4-amino-6-methyl- | H | H | 373.4 | 374.4 |
| | | 5-yl | 1,3,5-triazin-2-yl | | | | |
| 53 | —NH— | 4- | 4-amino-6-methyl- | H | H | 388.4 | 389.2 |
| | | ethylcarbonylamino- | 1,3,5-triazin-2-yl | | | | |
| | | phenyl | | | | | |
| 54 | —NH— | 3-hydroxyphenyl | 2-methylpyrimidin-4- | H | H | 317.3 | 318.3 |
| | | | yl | | | | |
| 55 | —NH— | 3-methylphenyl | 4-amino-6-methyl- | H | H | 331.4 | 332 |
| | | | 1,3,5-triazin-2-yl | | | | |
| 56 | —NH— | 3-methylpyrazol-5- | 4-amino-6-methyl- | 5-CH₃ | 6-CH₃ | 349.4 | 350.4 |
| | | yl | 1,3,5-triazin-2-yl | | | | |
| 57 | —NH— | 3-isopropylpyrazol- | 4-amino-6-methyl- | H | H | 349.4 | 350.2 |
| | | 5-yl | 1,3,5-triazin-2-yl | | | | |
| 58 | —NH— | 3-chlorophenyl | 4-amino-6-methyl- | H | H | 351.8 | 352; |
| | | | 1,3,5-triazin-2-yl | | | | 354 |
| 59 | —NH— | 3-fluorophenyl | 4-amino-6-methyl- | H | H | 335.3 | 336 |
| | | | 1,3,5-triazin-2-yl | | | | |
| 60 | —NH— | 3-thiophen-2-yl- | 4-amino-6-methyl- | H | H | 389.4 | 390.2 |
| | | pyrazol-5-yl | 1,3,5-triazin-2-yl | | | | |
| 61 | —NH— | tetrahydrofuran-3-yl | 4-amino-6-methyl- | H | H | 311.3 | 312.3 |
| | | | 1,3,5-triazin-2-yl | | | | |
| 62 | —NH— | cyclopropyl | 4-amino-6-methyl- | H | H | 281.3 | 282.3 |
| | | | 1,3,5-triazin-2-yl | | | | |
| 63 | —NH— | 1,2,4-triazol-3-yl | 4-amino-6-methyl- | H | H | 308.3 | 309.3 |
| | | | 1,3,5-triazin-2-yl | | | | |
| 64 | —NH— | 5-hydroxypyridin-3- | 4-amino-6-methyl- | H | H | 334.3 | 335.4 |
| | | yl | 1,3,5-triazin-2-yl | | | | |
| 65 | —NH— | 5-methyl-1.2.4- | 4-amino-6-methyl- | H | H | 322.3 | 323.4 |
| | | triazol-3-yl | 1,3,5-triazol-2-yl | | | | |
| 66 | —NH— | tetrahydropyran-4-yl | 4-amino-6-methyl- | H | H | 325.4 | 362.2 |
| | | | 1,3,5-triazin-2-yl | | | | |
| 67 | —NH— | 7-azaindazol-3-yl | 4-amino-6-methyl- | H | H | 358.4 | 359.2 |
| | | | 1,3,5-triazin-2-yl | | | | |
| 68 | —NH— | 1-methylindazol-3- | 4-amino-6-methyl- | H | H | 371.4 | 372.2 |
| | | yl | 1,3,5-triazin-2-yl | | | | |
| 69 | —NH— | 2-hydroxypyridin-5- | 4-amino-6-methyl- | H | H | 334.3 | 335.4 |
| | | yl | 1,3,5-triazin-2-yl | | | | |
| 70 | —NH— | 3-cyanophenyl | 4-amino-6-methyl- | H | H | 342.4 | 343.2 |
| | | | 1,3,5-triazin-2-yl | | | | |
| 71 | —NH— | 4- | 4-amino-6-methyl- | H | H | 374.4 | 375.2 |
| | | acetylaminophenyl | 1,3,5-triazin-2-yl | | | | |
| 72 | —NH— | 2,3-dihydroinden-2- | 4-amino-6-methyl- | H | H | 357.4 | 358.2 |
| | | yl | 1,3,5-triazin-2-yl | | | | |
| 73 | —NH— | 3-hydroxypyrazol-5- | 4-amino-6-methyl- | H | H | 323.3 | 324 |
| | | yl | 1,3,5-triazin-2-yl | | | | |
| 74 | —NH— | 1- | 4-amino-6-methyl- | H | H | 411.5 | 412.2 |
| | | cyclopropylmethyl- | 1,3,5-triazin-2-yl | | | | |
| | | indazol-3-yl | | | | | |
| 75 | —NH— | 2,3-dihydroinden-1- | 4-amino-6-methyl- | H | H | 357.4 | 358.2 |
| | | yl | 1,3,5-triazin-2-yl | | | | |
| 76 | —NH— | 3-hydroxyphenyl | 4-amino-6-methyl- | 5-Cl | 6-Cl | 402.2 | 402 |
| | | | 1,3,5-triazin-2-yl | | | | |
| 77 | —NH— | 4-cyanopyrazol-5-yl | 4-amino-6-methyl- | H | H | 332.3 | 333.3 |
| | | | 1,3,5-triazin-2-yl | | | | |
| 78 | —NH— | 1-methylpyrazol-5- | 4-amino-6-methyl- | H | H | 321.3 | 322.2 |
| | | yl | 1,3,5-triazin-2-yl | | | | |
| 79 | —NH— | 4-ethoxycarbonyl- | 4-amino-6-methyl- | H | H | 379.4 | 380.2 |
| | | pyrazol-5-yl | 1,3,5-triazin-2-yl | | | | |
| 80 | —NH— | 3-methoxycarbonyl- | 4-amino-6-methyl- | H | H | 375.4 | 376 |
| | | phenyl | 1,3,5-triazin-2-yl | | | | |
| 81 | —O— | 3-aminopyrazol-5-yl | 4-amino-6-methyl- | H | H | 323.3 | 324 |
| | | | 1,3,5-triazin-2-yl | | | | |
| 82 | —O— | 3-methoxyphenyl | 4-amino-6-methyl- | H | H | 348.4 | 349.3 |
| | | | 1,3,5-triazin-2-yl | | | | |
| 83 | —NH— | imidazol-2-yl | 4-amino-6-methyl- | H | H | 307.3 | 308.4 |
| | | | 1,3,5-triazin-2-yl | | | | |
| 84 | —NH— | isoindolin-1-one-4- | 4-amino-6-methyl- | H | H | 372.4 | 373.2 |
| | | yl | 1,3,5-triazin-2-yl | | | | |
| 85 | —NH— | 1,3-thiazol-2-yl | 4-amino-6-methyl- | H | H | 324.4 | 325.2 |
| | | | 1,3,5-triazin-2-yl | | | | |
| 86 | —NH— | 3-hydroxy-4- | 4-amino-6-methyl- | H | H | 375.4 | 376.2 |
| | | acetylphenyl | 1,3,5-triazin-2-yl | | | | |
| 87 | —NH— | 1-methoxycarbonyl- | 4-amino-6-methyl- | H | H | 339.4 | 340.2 |
| | | cyclopropyl | 1,3,5-triazin-2-yl | | | | |
| 88 | —NH— | 3-tert-butylpyrazol- | 4-amino-6-methyl- | H | H | 363.4 | 364.2 |
| | | 5-yl | 1,3,5-triazin-2-yl | | | | |
| 89 | —NH— | 5- | 4-amino-6-methyl- | H | H | 425.4 | 425.2 |
| | | trifluoromethylindazol- | 1,3,5-triazin-2-yl | | | | |
| | | 3-yl | | | | | |
| 90 | —NH— | 1-(4-chlorophenyl)- | 4-amino-6-methyl- | H | H | 391.9 | 392.4 |
| | | cyclopropyl | 1,3,5-triazin-2-yl | | | | |
| 91 | —NH— | 3-methoxyphenyl | 4-amino-6-methyl- | H | 6- | 405.4 | 460 |
| | | | 1,3,5-triazin-2-yl | | COOCH₃ | | |
| 92 | —NH— | 3- | 4-amino-6-methyl- | H | H | 389.5 | 390.3 |
| | | cyclohexylpyrazol- | 1,3,5-triazin-2-yl | | | | |
| | | 5-yl | | | | | |
| 93 | —NH— | 3-hydroxyphenyl | 6-amino-2- | H | H | 332.4 | 333.2 |
| | | | methylpyrimidin-4-yl | | | | |
| 94 | —NH— | 3-hydroxyphenyl | 6-pyrimidin-5- | H | H | 410.2 | 411.3 |
| | | | ylamino-2- | | | | |
| | | | methylpyrimidin-4-yl | | | | |
| 95 | —NH— | pyrazol-3-yl | 6-amino-2- | H | H | 306.3 | 307.4 |
| | | | methylpyrimidin-4-yl | | | | |
| 96 | —NH— | 3-hydroxyphenyl | 6-pyrimidin-2- | H | H | 410.2 | 411.3 |
| | | | ylamino-2- | | | | |
| | | | methylpyrimidin-4-yl | | | | |
| 97 | —NH— | 3- | 6-amino-2- | H | H | 346.4 | 347.4 |
| | | cyclopropylpyrazol- | methylpyrimidin-4-yl | | | | |
| | | 5-yl | | | | | |
| 98 | —O— | 3-hydroxyphenyl | 2-amino-6- | H | H | 333.3 | 334.2 |
| | | | methylpyrimidin-4-yl | | | | |
| 99 | —O— | 3-methoxyphenyl | 2-amino-6- | H | H | 347.4 | 348.4 |
| | | | methylpyrimidin-4-yl | | | | |
| 100 | —NH— | pyrazol-5-yl | 4-amino-6-methyl- | 5-Cl | 6-Cl | 376.2 | 375.9 |
| | | | 1,3,5-triazin-2-yl | | | | |
| 101 | —NH— | 4-[4- | 4-amino-6-methyl- | H | H | 497.5 | 498.2 |
| | | methoxyphenyl- | 1,3,5-triazin-2-yl | | | | |
| | | NHCSNH—]phenyl | | | | | |
| 102 | —NH— | 4-[3- | 4-amino-6-methyl- | H | H | 497.5 | 498.2 |
| | | methoxyphenyl- | 1,3,5-triazin-2-yl | | | | |
| | | NHCSNH—]phenyl | | | | | |
| 103 | —NH— | pyrazol-5-yl | 4-amino-6-methyl- | 4-CH₃ | H | 321.3 | 322.2 |
| | | | 1,3,5-triazin-2-yl | | | | |
| 104 | —NH— | 2,6-dichlorophenyl- | 4-amino-6-methyl- | H | H | 541.4 | 541 |
| | | sulfonyl- | 1,3,5-triazin-2-yl | | | | |
| | | aminophenyl | | | | | |
| 105 | —NH— | pyrazol-3-yl | 4-cyanomethylamino- | H | H | 346.3 | 347.2 |
| | | | 6-methyl-1,3,5-triazin- | | | | |
| | | | 2-yl | | | | |
| 106 | —NH— | isoxazol-3-yl | 4-methylamino-6- | H | H | 322.3 | 323.2 |
| | | | methyl-1,3,5-triazin-2- | | | | |
| | | | yl | | | | |
| 107 | —NH— | isoxazol-3-yl | 4-cyanomethylamino- | H | H | 347.3 | 348.2 |
| | | | 6-methyl-1,3,5-triazin- | | | | |
| | | | 2-yl | | | | |
| 108 | —NH— | 4-[ethylNHCSNH—]- | 4-amino-6-methyl- | H | H | 419.5 | 420.3 |
| | | phenyl | 1,3,5-triazin-2-yl | | | | |
| 109 | —NH— | 3-[3,5- | 4-amino-6-methyl- | H | H | 536.4 | 536 |
| | | dichlorophenyl- | 1,3,5-triazin-2-yl | | | | |
| | | NHCSNH—]phenyl | | | | | |
| 110 | —NH— | 4-[2- | 4-amino-6-methyl- | H | H | 540.5 | 541 |
| | | trifluoromethyl- | 1,3,5-triazin-2-yl | | | | |
| | | phenyl- | | | | | |
| | | sulfonylamino]]- | | | | | |
| | | phenyl | | | | | |
| 111 | —NH— | 4-[—NHCONHCH₃]- | 4- | H | H | 428.45 | 429.2 |
| | | phenyl | ((cyanomethyl)amino)- | | | | |
| | | | 6-methyl-1,3,5- | | | | |
| | | | triazin-2-yl | | | | |
| 112 | —NH— | 4-[—NHCONHCH2CH3]- | 4- | H | H | 442.45 | 443.1 |
| | | phenyl | 6-methyl-1,3,5- | | | | |
| | | | triazin-2-yl | | | | |
| 113 | —NH— | pyrazol-3-yl | 4-(methylamino)-6- | 6- | H | 399.41 | 400.2 |
| | | | methyl-1,3,5-triazin-2- | pyrimidin- | | | |
| | | | yl | 5-yl | | | |
| 115 | —NH— | 4-[—NHCONHpyridin-4- | 4-amino-6-methyl- | H | H | 452.47 | 453.3 |
| | | yl]phenyl | 1,3,5-triazin-2-yl | | | | |
| 116 | —NH— | pyrazol-3-yl | 4-(methylamino)-6- | mixture of | H | 412.45 | 413.4 |
| | | | methyl-1,3,5-triazin-2- | 5- and 6-(2- | | | |
| | | | yl | methylpyridin- | | | |
| | | | | 4-yl) | | | |
| 117 | —NH— | pyrazol-5-yl | 4-amino-6-methyl- | mixture of | H | 373.38 | 374.4 |
| | | | 1,3,5-triazin-2-yl | 5- and 6- | | | |
| | | | | pyrazol-4-yl | | | |
| 118 | —NH— | pyrazol-3-yl | 4-methylamino-6- | mixture of 5 | H | 399.41 | 400.0 |
| | | | methyl-1,3,5-triazin-2- | and 6- | | | |
| | | | yl | pyrimidin- | | | |
| | | | | 5-yl | | | |
| 119 | —NH— | 4-methylcarbonyl- | 4-methylamino-6- | H | H | 388.43 | 389.2 |
| | | aminophenyl | methyl-1,3,5-triazin-2- | | | | |
| | | | yl | | | | |
| 120 | —NH— | pyrazol-5-yl | 4-amino-6-methyl- | mixture of | H | 384.4 | 385.2 |
| | | | 1,3,5-triazin-2-yl | 5- and 6- | | | |
| | | | | pyridin-4-yl | | | |
| 121 | —NH— | pyrazol-5-yl | 4-amino-6-methyl- | 6-pyridin-3- | H | 384.4 | 385.2 |
| | | | 1,3,5-triazin-2-yl | yl | | | |
| 122 | —NH— | pyrazol-3-yl | 4-methylamino-6- | mixture of | H | 402.46 | 403.0 |
| | | | methyl-1,3,5-triazin-2- | 5- and 6- | | | |
| | | | yl | (1,2,3,6- | | | |
| | | | | tetrahydropyridin- | | | |
| | | | | 4-yl | | | |
| 123 | —NH— | pyrazol-5-yl | 4-amino-6-methyl- | 6-pyridin-4- | H | 384.4 | 385.2 |
| | | | 1,3,5-triazin-2-yl | yl | | | |
| 124 | —NH— | pyrazol-5-yl | 4-amino-6-methyl- | mixture of | H | 443.46 | 444.3 |
| | | | 1,3,5-triazin-2-yl | 5- and 6-(3- | | | |
| | | | | hydroxymethyl-4- | | | |
| | | | | 4- | | | |
| | | | | methoxy-) | | | |
| | | | | phenyl | | | |
| 125 | —NH— | pyrazol-3-yl | 4-methylamino-6- | mixture of | H | 454.49 | 455.0 |
| | | | methyl-1,3,5-triazin-2- | 5- and 6- | | | |
| | | | yl | (4-methyl- | | | |
| | | | | aminocarbonyl)- | | | |
| | | | | phenyl | | | |
| 126 | —NH— | pyrazol-3-yl | 4-methylamino-6- | mixture of | H | 472.48 | 473.0 |
| | | | methyl-1,3,5-triazin-2- | 5- and 6-(3- | | | |
| | | | yl | F-4-methylamino- | | | |
| | | | | carbonyl) | | | |
| | | | | phenyl | | | |
| 127 | —NH— | 4-methylcarbonyl | 4- | H | H | 413.44 | 414.2 |
| | | aminophenyl | (cyanomethylamino)- | | | | |
| | | | 6-methyl-1,3,5-triazin- | | | | |
| | | | 2-yl | | | | |
| 128 | —NH— | pyrazol-3-yl | 4- | mixture of | H | 482.50 | 483.0 |
| | | | (cyanomethylamino)- | 5- and 6-(3- | | | |
| | | | 6-methyl-1,3,5-triazin- | hydroxymethyl-4- | | | |
| | | | 2-yl | methoxy)phenyl | | | |
| 129 | —NH— | pyrazol-3-yl | 4-methylamino-6- | mixture of | H | 454.49 | 455.0 |
| | | | methyl-1,3,5-triazin-2- | 5- and 6-(4-methyl- | | | |
| | | | yl | carbonylamino) | | | |
| | | | | phenyl | | | |
| 130 | —NH— | pyrazol-3-yl | 4-methylamino-6- | mixture of | H | 398.42 | 399.3 |
| | | | methyl-1,3,5-triazin-2- | 5- and 6- | | | |
| | | | yl | pyridin-3-yl | | | |
| 131 | —NH— | pyrazol-5-yl | 4-amino-6-methyl- | mixture of | H | 323.31 | 324.3 |
| | | | 1,3,5-triazin-2-yl | 5- and 6- | | | |
| | | | | hydroxy | | | |
| 132 | —NH— | pyrazol-3-yl | 4-cyanomethylamino- | mixture of | H | 423.43 | 424.2 |
| | | | 6-methyl-1,3,5-triazin- | 5- and 6- | | | |
| | | | 2-yl | pyridin-4-yl | | | |
| 133 | —NH— | pyrazol-5-yl | 4-amino-6-methyl- | mixture of | H | 337.34 | 338.0 |
| | | | 1,3,5-triazin-2-yl | 5- and 6- | | | |
| | | | | methoxy | | | |
| 134 | —NH— | pyrazol-3-yl | 4-methylamino-6- | mixture of | H | 457.50 | 458.0 |
| | | | methyl-1,3,5-triazin-2- | 5- and 6-(3- | | | |
| | | | yl | hydroxy- | | | |
| | | | | methyl-4- | | | |
| | | | | methoxy)phenyl | | | |
| 135 | —NH— | 2-aminopyrimidin- | 4-amino-6-methyl- | H | H | 334.34 | 335.1 |
| | | 5-yl | 1,3,5-triazin-2-yl | | | | |
| 136 | —NH— | pyrazol-3-yl | 4-cyanomethylamino- | 5-methyl | 6-CH₃ | 374.40 | 375.3 |
| | | | 6-methyl-1,3,5-triazin- | | | | |
| | | | 2-yl | | | | |
| 137 | —NH— | 4- | 4-amino-6-methyl- | H | H | 374.4 | 375.3 |
| | | methylaminocarbonyl- | 1,3,5-triazin-2-yl | | | | |
| | | phenyl | | | | | |
| 138 | —NH— | pyrazol-3-yl | 4-methylamino-6- | 5-methyl | 6-CH₃ | 349.39 | 350.4 |
| | | | methyl-1,3,5-triazin-2-yl | | | | |
| 139 | —NH— | pyrazol-3-yl | 4-methylamino-6- | mixture of | H | 398.42 | 399.3 |
| | | | methyl-1,3,5-triazin-2- | 5- and 6- | | | |
| | | | yl | pyridin-4-yl | | | |
| 140 | —NH— | pyrazol-5-yl | 4-amino-6-methyl- | 5-(3- | H | 443.46 | 444.3 |
| | | | 1,3,5-triazin-2-yl | hydroxy- | | | |
| | | | | methyl-4- | | | |
| | | | | methoxy)phenyl | | | |
| 141 | —NH— | 4- | 4-amino-6-methyl- | H | H | 400.43 | 401.2 |
| | | cyclopropylcarbonyl- | 1,3,5-triazin-2-yl | | | | |
| | | amino-phenyl | | | | | |
| 142 | —NH— | pyrazol-3-yl | 4-methylamino-6- | mixture of | H | 427.46 | 428.0 |
| | | | methyl-1,3,5-triazin-2- | 5- and 6-(4- | | | |
| | | | yl | methoxy)- | | | |
| | | | | phenyl | | | |
| 143 | —NH— | pyrazol-3-yl | 4-(2- | H | H | 360.38 | 361.2 |
| | | | cyanoethylamino)-6- | | | | |
| | | | methyl-1,3,5-triazin-2- | | | | |
| | | | yl | | | | |
| 144 | —NH— | pyrazol-3-yl | 4-(2-hydroxyethyl- | H | H | 351.37 | 352.0 |
| | | | amino)-6-methyl- | | | | |
| | | | 1,3,5-triazin-2-yl | | | | |
| 145 | —NH— | pyrazol-5-yl | 4-methylamino-6- | 5-F | 6-F | 357.32 | 358 |
| | | | methyl-1,3,5-triazin-2- | | | | |
| | | | yl | | | | |
| 146 | —NH— | pyrazol-5-yl | 4-amino-6-methyl- | 5-pyridin-4- | H | 384.4 | 385.2 |
| | | | 1,3,5-triazin-2-yl | yl | | | |
| 147 | —NH— | pyrazol-3-yl | 4-methylamino-6- | mixture of | H | 415.43 | 416.0 |
| | | | methyl-1,3,5-triazin-2- | 5- and 6-(4- | | | |
| | | | yl | fluorophenyl)- | | | |
| 148 | —NH— | 3-F-4- | 4-amino-6-methyl- | H | H | 351.34 | 352.2 |
| | | hydroxyphenyl | 1,3,5-triazin-2-yl | | | | |
| 149 | —NH— | 1H-2- | 4-amino-6-methyl- | H | H | 373.37 | 374.1 |
| | | oxobenzimidazol-5-yl | 1,3,5-triazin-2-yl | | | | |
| 150 | —NH— | pyrazol-3-yl | 4-methylamino-6- | 5- | H | 399.41 | 400.2 |
| | | | methyl-1,3,5-triazin-2- | pyrimidin- | | | |
| | | | yl | 5-yl | | | |
| 151 | —NH— | pyrazol-3-yl | 4-(2-methoxyethyl- | H | H | 365.39 | 366.0 |
| | | | amino)-6-methyl- | | | | |
| | | | 1,3,5-triazin-2-yl | | | | |
| 152 | —NH— | pyrazol-5-yl | 4-cyclopropylamino- | H | H | 347.38 | 348.3 |
| | | | 6-methyl-1,3,5-triazin- | | | | |
| | | | 2-yl | | | | |
| 153 | —NH— | 6-methoxypyridin-3- | 4-amino-6-methyl- | H | H | 348.36 | 349.2 |
| | | yl | 1,3,5-triazin-2-yl | | | | |
| 154 | —NH— | 4- | 4-amino-6-methyl- | H | H | 388.43 | 389.4 |
| | | (ethylaminocarbonyl)- | 1,3,5-triazin-2-yl | | | | |
| | | phenyl | | | | | |
| 155 | —NH— | pyrazol-3-yl | 4-benzylamino-6- | H | H | 397.44 | 398.1 |
| | | | methyl-1,3,5-triazin-2-yl | | | | |
| 156 | —NH— | 1H-indazol-4-yl | 4-amino-6-methyl- | H | H | 357.37 | 358.1 |
| | | | 1,3,5-triazin-2-yl | | | | |
| 157 | —NH— | pyrazol-3-yl | 4-(2-piperazin-1- | H | H | 419.49 | 420.0 |
| | | | ylethylamino)-6- | | | | |
| | | | methyl-1,3,5-triazin-2- | | | | |
| | | | yl | | | | |
| 158 | —NH— | pyrazol-3-yl | 4-(2-morpholin-4- | H | H | 420.47 | 421.2 |
| | | | ylethylamino)-6- | | | | |
| | | | methyl-1,3,5-triazin-2-yl | | | | |
| 159 | —NH— | 4-(pyridin-4- | 4-amino-6-methyl- | H | H | 437.46 | 438.3 |
| | | ylaminocarbonyl)- | 1,3,5-triazin-2-yl | | | | |
| | | phenyl | | | | | |
| 160 | —NH— | pyrazol-3-yl | 4-ethylamino-6- | H | H | 335.37 | 336.3 |
| | | | methyl-1,3,5-triazin-2- | | | | |
| | | | yl | | | | |
| 161 | —NH— | 3- | 4-amino-6-methyl- | H | H | 374.4 | 375.1 |
| | | methylcarbonylamino- | 1,3,5-triazin-2-yl | | | | |
| | | phenyl | | | | | |
| 162 | —NH— | pyrazol-3-yl | 4-(tetrahydropyran-4- | H | H | 405.46 | 406.2 |
| | | | ylmethylamino)-6- | | | | |
| | | | methyl-1,3,5-triazin-2-yl | | | | |
| 163 | —NH— | pyrazol-3-yl | 4-(tetrahydropyran-4- | H | H | 391.43 | 392.2 |
| | | | ylamino)-6-methyl- | | | | |
| | | | 1,3,5-triazin-2-yl | | | | |
| 164 | —NH— | 4-(2,3-dihydro-1- | 4-amino-6-methyl- | H | H | 478.51 | 479.2 |
| | | benzofuran-5- | 1,3,5-triazin-2-yl | | | | |
| | | ylcarbonylamino) | | | | | |
| | | phenyl | | | | | |
| 165 | —NH— | pyrazol-3-yl | 4-(2-propylamino)-6- | H | H | 349.39 | 350.2 |
| | | | methyl-1,3,5-triazin-2-yl | | | | |
| | | | yl | | | | |
| 166 | —NH— | pyrazol-3-yl | 4-(imidazol-2- | H | H | 387.40 | 388.0 |
| | | | ylmethyl-amino)-6- | | | | |
| | | | methyl-1,3,5-triazin-2-yl | | | | |
| 167 | —NH— | 4-aminophenyl | 4-amino-6-methyl- | H | H | 332.36 | 333.2 |
| | | | 1,3,5-triazin-2-yl | | | | |
| 168 | —NH— | 4-(3-dimethylamino- | 4-amino-6-methyl- | H | H | 479.54 | 480.2 |
| | | phenyl- | 1,3,5-triazin-2-yl | | | | |
| | | carbonylamino)- | | | | | |
| | | phenyl | | | | | |
| 169 | —NH— | 1-methylpyrazol-4- | 4-amino-6-methyl- | H | H | 321.34 | 322.2 |
| | | yl | 1,3,5-triazin-2-yl | | | | |
| 170 | —NH— | 4- | 4-amino-6-methyl- | H | H | 428.49 | 429.3 |
| | | (cyclopentylcarbonyl- | 1,3,5-triazin-2-yl | | | | |
| | | amino)-phenyl | | | | | |
| 171 | —NH— | 4-(tert- | 4-amino-6-methyl- | H | H | 416.48 | 417.2 |
| | | butylcarbonylamino)- | 1,3,5-triazin-2-yl | | | | |
| | | phenyl | | | | | |
| 172 | —NH— | pyrazol-3-yl | 4-(cyclopropylmethyl- | H | H | 361.40 | 362.4 |
| | | | amino)-6-methyl- | | | | |
| | | | 1,3,5-triazin-2-yl | | | | |
| 173 | —NH— | pyrimidin-2-yl | 4-methyl-1,3,5-triazin- | H | H | 319.32 | 320.2 |
| | | | 2-yl | | | | |
| 174 | —NH— | pyrazol-3-yl | 4-amino-6-methyl- | H | H | 292.30 | 293.0 |
| | | | 1,3,5-triazin-2-yl | | | | |
| 175 | —NH— | 4-(2.1.3- | 4-amino-6-methyl- | H | H | 494.53 | 495.2 |
| | | benzothiadiazol-4- | 1,3,5-triazin-2-yl | | | | |
| | | ylcarbonylamino) | | | | | |
| | | phenyl | | | | | |
| 176 | —NH— | pyrazol-3-yl | 4-(pyrimidin-2- | H | H | 385.39 | 386.0 |
| | | | ylamino)-6-methyl- | | | | |
| | | | 1,3,5-triazin-2-yl | | | | |
| 177 | —NH— | 4-(2- | 4-amino-6-methyl- | H | H | 402.45 | 403.2 |
| | | isopropylamino- | 1,3,5-triazin-2-yl | | | | |
| | | carbonyl)-phenyl | | | | | |
| 178 | —NH— | 3-methylphenyl | 4-amino-6-methyl- | H | H | 331.4 | |
| | | | 1,3,5-triazin-2-yl | | | | |
| 179 | —NH— | 1-(2,2,2- | 4-amino-6-methyl- | H | H | 389.34 | 390.2 |
| | | trifluoroethyl)- | 1,3,5-triazin-2-yl | | | | |
| | | pyrazol-3-yl | | | | | |
| 180 | —NH— | pyrimidin-4-yl | 4-amino-6-methyl- | H | H | 319.32 | 320.2 |
| | | | 1,3,5-triazin-2-yl | | | | |
| 181 | —NH— | 3- | 4-amino-6-methyl- | H | H | 364.37 | 365.2 |
| | | (methylaminocarbonyl)- | 1,3,5-triazin-2-yl | | | | |
| | | yl)-pyrazol-5-yl | | | | | |
| 182 | —NH— | 4-(3,5-diClphenyl- | 4-(cyanomethyl- | H | H | 575.48 | 575.0 |
| | | NHCSNH—)phenyl | amino)-6-methyl- | | | | |
| | | | 1,3,5-triazin-2-yl | | | | |
| 183 | —NH— | pyrazin-2-yl | 4-amino-6-methyl- | H | H | 319.32 | 320.2 |
| | | | 1,3,5-triazin-2-yl | | | | |
| 184 | —NH— | 3-cyanomethyl | 4-amino-6-methyl- | H | H | 342.36 | 343.2 |
| | | | 1,3,5-triazin-2-yl | | | | |
| 185 | —NH— | pyridazin-4-yl | 4-amino-6-methyl- | H | H | 319.32 | 319.9 |
| | | | 1,3,5-triazin-2-yl | | | | |
| 186 | —NH— | 2-oxopyrrolidin-4yl | 4-amino-6-methyl- | H | H | 324.34 | 325.1 |
| | | | 1,3,5-triazin-2-yl | | | | |
| 187 | —NH— | 3- | 4-amino-6-methyl- | H | H | 390.40 | 391.2 |
| | | (cyclopropylamino- | 1,3,5-triazin-2-yl | | | | |
| | | carbonyl)-pyrazol-5- | | | | | |
| | | yl | | | | | |
| 188 | —NH— | cyclopentyl | 4-amino-6-methyl- | H | H | 309.37 | 310.1 |
| | | | 1,3,5-triazin-2-yl | | | | |
| 189 | —NH— | 3-cyanophenyl | 4-amino-6-methyl- | H | H | 342.36 | 343.2 |
| | | | 1,3,5-triazin-2-yl | | | | |
| 190 | —NH— | pyridazin-3-yl | 4-amino-6-methyl- | H | H | 319.32 | 320.1 |
| | | | 1,3,5-triazin-2-yl | | | | |
| 191 | —NH— | cyclohexyl | 4-amino-6-methyl- | H | H | 323.4 | 324.2 |
| | | | 1,3,5-triazin-2-yl | | | | |
| 1902 | —NH— | 2-oxopyrrolidin-3yl | 4-amino-6-methyl- | H | H | 324.34 | 325.1 |
| | | | 1,3,5-triazin-2-yl | | | | |
| 193 | —NH— | pyrrolidin-3yl | 4-amino-6-methyl- | H | H | 310.36 | 311.1 |
| | | | 1,3,5-triazin-2-yl | | | | |
| 194 | —NH— | pyrazol-5-yl | 4-dimethylamino-6- | H | H | 335.37 | 336.0 |
| | | | methyl-1,3,5-triazin-2-yl | | | | |
| 195 | —NH— | 4-(4-tert- | 4-amino-6-methyl- | H | H | 492.58 | 493.2 |
| | | butylphenyl- | 1,3,5-triazin-2-yl | | | | |
| | | carbonyl- | | | | | |
| | | amino)phenyl | | | | | |
| 196 | —NH— | 4- | 4-amino-6-methyl- | H | H | 442.52 | 443.2 |
| | | (cyclohexylcarbonyl- | 1,3,5-triazin-2-yl | | | | |
| | | amino)phenyl | | | | | |
| 197 | —NH— | 4-(3- | 4-amino-6-methyl- | H | H | 504.47 | 505.2 |
| | | CF₃phenylcarbonyl- | 1,3,5-triazin-2-yl | | | | |
| | | amino)phenyl | | | | | |
| 198 | —NH— | 4-(4- | 4-amino-6-methyl- | H | H | 520.47 | 521.2 |
| | | OCF₃phenylcarbonyl | 1,3,5-triazin-2-yl | | | | |
| | | amino)phenyl | | | | | |
| 199 | —NH— | 3- | 4-amino-6-methyl- | H | H | 379.38 | 380.4 |
| | | ethoxycarbonylpyrazol- | 1,3,5-triazin-2-yl | | | | |
| | | 5-yl | | | | | |
| 200 | —NH— | 4-(4- | 4-amino-6-methyl- | H | H | 490.51 | 491.2 |
| | | fluorophenylsulfonyl- | 1,3,5-triazin-2-yl | | | | |
| | | amino)phenyl | | | | | |
| 201 | —NH— | 1-(morpholin-4- | 4-amino-6-methyl- | H | H | 420.43 | 421.2 |
| | | ylcarbonyl)-pyrazol- | 1,3,5-triazin-2-yl | | | | |
| | | 4-yl | | | | | |
| 202 | —NH— | 1,3-benzoxazol-6-yl | 4-amino-6-methyl- | H | H | 358.36 | 359.4 |
| | | | 1,3,5-triazin-2-yl | | | | |
| 203 | —NH— | 1-(pyridin-4- | 4-amino-6-methyl- | H | H | 427.42 | 428.2 |
| | | ylaminocarbonyl)- | 1,3,5-triazin-2-yl | | | | |
| | | pyrazol-4-yl | | | | | |

and are named as:
5-((1-(4-amino-6-methyl-1,3,5-triazin-2-yl)-1H-benzimidazol-2-yl)amino)-1,3-benzenediol;
1-(4-amino-6-methyl-1,3,5-triazin-2-yl)-2-((3-hydroxyphenyl)amino)-1H-benzimidazole-6-carboxylic acid;
3-((1-(4-amino-6-methyl-1,3,5-triazin-2-yl)-1H-benzimidazol-2-yl)amino)phenol;
1-(4-((1-(4-amino-6-methyl-1,3,5-triazin-2-yl)-1H-benzimidazol-2-yl)amino)phenyl)-3-methylurea;
1-(4-((1-(4-amino-6-methyl-1,3,5-triazin-2-yl)-1H-benzimidazol-2-yl)amino)phenyl)-3-ethylurea;
((4-(2-((3-hydroxyphenyl)amino)-1H-benzimidazol-1-yl)-6-methyl-1,3,5-triazin-2-yl)amino)acetonitrile;
1-(4-((1-(4-amino-6-methyl-1,3,5-triazin-2-yl)-1H-benzimidazol-2-yl)amino)phenyl)-3-(3-hydroxyphenyl)urea;
methyl 3-((1-(4-amino-6-methyl-1,3,5-triazin-2-yl)-1H-benzimidazol-2-yl)amino)-5-hydroxybenzoate;
1-(4-amino-6-methyl-1,3,5-triazin-2-yl)-5,6-difluoro-N-1H-pyrazol-5-yl-1H-benzimidazol-2-amine;
1-(4-amino-6-methyl-1,3,5-triazin-2-yl)-N-1H-pyrazol-3-yl-1H-benzimidazol-2-amine;
Mixture of 1-(4-amino-6-methyl-1,3,5-triazin-2-yl)-5-fluoro-N-1H-pyrazol-5-yl-1H-benzimidazol-2-amine and 1-(4-amino-6-methyl-1,3,5-triazin-2-yl)-6-fluoro-N-1H-pyrazol-5-yl-1H-benzimidazol-2-amine;
5-((1-(4-amino-6-methyl-1,3,5-triazin-2-yl)-1H-benzimidazol-2-yl)amino)-2-methoxyphenol;
N-(1-(4-amino-6-methyl-1,3,5-triazin-2-yl)-1H-benzimidazol-2-yl)-1H-indazol-6-amine;
1-(4-amino-6-methyl-1,3,5-triazin-2-yl)-N-1H-indol-6-yl-1H-benzimidazol-2-amine;
1-(4-amino-6-methyl-1,3,5-triazin-2-yl)-N-(3-cyclopropyl-1H-pyrazol-5-yl)-1H-benzimidazol-2-amine;
1-(4-((1-(4-amino-6-methyl-1,3,5-triazin-2-yl)-1H-benzimidazol-2-yl)amino)phenyl)-3-(3-methoxyphenyl)urea;
methyl 4-(((4-((1-(4-amino-6-methyl-1,3,5-triazin-2-yl)-1H-benzimidazol-2-yl)amino)phenyl)carbamoyl)amino)benzoate;
3-((1-(4-amino-6-methyl-1,3,5-triazin-2-yl)-5,6-dimethyl-1H-benzimidazol-2-yl)amino)phenol;
N˜5˜-(1-(4-amino-6-methyl-1,3,5-triazin-2-yl)-1H-benzimidazol-2-yl)-2,5-pyridinediamine;
N-(1-(4-amino-6-methyl-1,3,5-triazin-2-yl)-1H-benzimidazol-2-yl)-1H-indazol-3-amine;
4-((1-(4-amino-6-methyl-1,3,5-triazin-2-yl)-1H-benzimidazol-2-yl)amino)-2-hydroxybenzoic acid;
1-(4-amino-6-methyl-1,3,5-triazin-2-yl)-N-1H-indol-4-yl-1H-benzimidazol-2-amine;
N-(1-(4-amino-6-methyl-1,3,5-triazin-2-yl)-1H-benzimidazol-2-yl)-4-fluoro-1H-indazol-3-amine;
4-((1-(4-amino-6-methyl-1,3,5-triazin-2-yl)-1H-benzimidazol-2-yl)amino)phenol;
3-((1-(4-amino-6-methyl-1,3,5-triazin-2-yl)-1H-benzimidazol-2-yl)amino)benzoic acid;
mixture of 1-(4-amino-6-methyl-1,3,5-triazin-2-yl)-5-bromo-N-1H-pyrazol-3-yl-1H-benzimidazol-2-amine and 1-(4-amino-6-methyl-1,3,5-triazin-2-yl)-6-bromo-N-1H-pyrazol-3-yl-1H-benzimidazol-2-amine;
1-(4-amino-6-methyl-1,3,5-triazin-2-yl)-5,6-dimethyl-N-1H-pyrazol-5-yl-1H-benzimidazol-2-amine;
1-(4-amino-6-methyl-1,3,5-triazin-2-yl)-N-3-pyridinyl-1H-benzimidazol-2-amine;
1-(4-amino-6-methyl-1,3,5-triazin-2-yl)-N-3-isoxazolyl-1H-benzimidazol-2-amine;
1-(4-amino-6-methyl-1,3,5-triazin-2-yl)-N-1H-pyrazol-4-yl-1H-benzimidazol-2-amine;
5-((1-(4-amino-6-methyl-1,3,5-triazin-2-yl)-1H-benzimidazol-2-yl)amino)-2-fluorophenol;
methyl 1-(4-amino-6-methyl-1,3,5-triazin-2-yl)-2-((3-methoxyphenyl)amino)-1H-benzimidazole-5-carboxylate;
3-((1-(4-amino-6-methyl-1,3,5-triazin-2-yl)-1H-benzimidazol-2-yl)amino)benzamide;
5-((1-(4-amino-6-methyl-1,3,5-triazin-2-yl)-1H-benzimidazol-2-yl)amino)-2-methylphenol;
(1-(4-amino-6-methyl-1,3,5-triazin-2-yl)-2-((3-methoxyphenyl)amino)-1H-benzimidazol-5-yl)methanol;
N-(4-((1-(4-amino-6-methyl-1,3,5-triazin-2-yl)-1H-benzimidazol-2-yl)amino)phenyl)-4-fluorobenzenesulfonamide;
N-(1-(4-amino-6-methyl-1,3,5-triazin-2-yl)-1H-benzimidazol-2-yl)-2,6-pyridinediamine;
6-((1-(4-amino-6-methyl-1,3,5-triazin-2-yl)-1H-benzimidazol-2-yl)amino)-2,3-dihydro-1H-inden-1-one;
1-(4-amino-6-methyl-1,3,5-triazin-2-yl)-N-(1-methyl-1H-pyrazol-3-yl)-1H-benzimidazol-2-amine;
1-(4-amino-6-methyl-1,3,5-triazin-2-yl)-N-(3-methoxyphenyl)-1H-benzimidazol-2-amine;
1-(4-amino-6-methyl-1,3,5-triazin-2-yl)-N-(3-cyclopropyl-1H-pyrazol-5-yl)-5,6-dimethyl-1H-benzimidazol-2-amine;
N-(4-((1-(4-amino-6-methyl-1,3,5-triazin-2-yl)-1H-benzimidazol-2-yl)amino)phenyl)-4-methylbenzenesulfonamide;
1-(4-amino-6-methyl-1,3,5-triazin-2-yl)-N-(3-methyl-5-isothiazolyl)-1H-benzimidazol-2-amine;
3-((1-(4-amino-6-methyl-1,3,5-triazin-2-yl)-1H-benzimidazol-2-yl)amino)benzenesulfonamide;
1-(4-amino-6-methyl-1,3,5-triazin-2-yl)-N-phenyl-1H-benzimidazol-2-amine;
N-(4-((1-(4-amino-6-methyl-1,3,5-triazin-2-yl)-1H-benzimidazol-2-yl)amino)phenyl)methanesulfonamide;

(1-(4-amino-6-methyl-1,3,5-triazin-2-yl)-2-(3-methoxyphenylamino)-1H-benzo[d]imidazol-6-yl)methanol;
methyl 4-((1-(4-amino-6-methyl-1,3,5-triazin-2-yl)-1H-benzimidazol-2-yl)amino)-2-hydroxybenzoate;
1-(4-amino-6-methyl-1,3,5-triazin-2-yl)-N-(5-methyl-1H-pyrazol-3-yl)-1H-benzimidazol-2-amine;
1-(4-methyl-6-(methylamino)-1,3,5-triazin-2-yl)-N-1H-pyrazol-3-yl-1H-benzimidazol-2-amine;
1-(4-amino-6-methyl-1,3,5-triazin-2-yl)-N-(3-(2-furanyl)-1H-pyrazol-5-yl)-1H-benzimidazol-2-amine;
N-(4-((1-(4-amino-6-methyl-1,3,5-triazin-2-yl)-1H-benzimidazol-2-yl)amino)phenyl)propanamide;
3-((1-(2-methyl-4-pyrimidinyl)-1H-benzimidazol-2-yl)amino)phenol;
1-(4-amino-6-methyl-1,3,5-triazin-2-yl)-N-(3-methylphenyl)-1H-benzimidazol-2-amine;
1-(4-amino-6-methyl-1,3,5-triazin-2-yl)-5,6-dimethyl-N-(3-methyl-1H-pyrazol-5-yl)-1H-benzimidazol-2-amine;
1-(4-amino-6-methyl-1,3,5-triazin-2-yl)-N-(3-(1-methylethyl)-1H-pyrazol-5-yl)-1H-benzimidazol-2-amine;
1-(4-amino-6-methyl-1,3,5-triazin-2-yl)-N-(3-chlorophenyl)-1H-benzimidazol-2-amine;
1-(4-amino-6-methyl-1,3,5-triazin-2-yl)-N-(3-fluorophenyl)-1H-benzimidazol-2-amine;
1-(4-amino-6-methyl-1,3,5-triazin-2-yl)-N-(3-(2-thiophenyl)-1H-pyrazol-5-yl)-1H-benzimidazol-2-amine;
1-(4-amino-6-methyl-1,3,5-triazin-2-yl)-N-((3RS)-tetrahydro-3-furanyl)-1H-benzimidazol-2-amine;
1-(4-amino-6-methyl-1,3,5-triazin-2-yl)-N-cyclopropyl-1H-benzimidazol-2-amine;
1-(4-amino-6-methyl-1,3,5-triazin-2-yl)-N-1H-1,2,4-triazol-3-yl-1H-benzimidazol-2-amine;
5-((1-(4-amino-6-methyl-1,3,5-triazin-2-yl)-1H-benzimidazol-2-yl)amino)-3-pyridinol;
1-(4-amino-6-methyl-1,3,5-triazin-2-yl)-N-(5-methyl-4H-1,2,4-triazol-3-yl)-1H-benzimidazol-2-amine;
1-(4-amino-6-methyl-1,3,5-triazin-2-yl)-N-(tetrahydro-2H-pyran-4-yl)-1H-benzimidazol-2-amine;
N-(1-(4-amino-6-methyl-1,3,5-triazin-2-yl)-1H-benzimidazol-2-yl)-1H-pyrazolo[3,4-b]pyridin-3-amine;
N-(1-(4-amino-6-methyl-1,3,5-triazin-2-yl)-1H-benzimidazol-2-yl)-1-methyl-1H-indazol-3-amine;
5-((1-(4-amino-6-methyl-1,3,5-triazin-2-yl)-1H-benzimidazol-2-yl)amino)-2-pyridinol;
3-((1-(4-amino-6-methyl-1,3,5-triazin-2-yl)-1H-benzimidazol-2-yl)amino)benzonitrile;
N-(4-((1-(4-amino-6-methyl-1,3,5-triazin-2-yl)-1H-benzimidazol-2-yl)amino)phenyl)acetamide;
1-(4-amino-6-methyl-1,3,5-triazin-2-yl)-N-(2,3-dihydro-1H-inden-2-yl)-1H-benzimidazol-2-amine;
5-((1-(4-amino-6-methyl-1,3,5-triazin-2-yl)-1H-benzimidazol-2-yl)amino)-1H-pyrazol-3-ol;
N-(1-(4-amino-6-methyl-1,3,5-triazin-2-yl)-1H-benzimidazol-2-yl)-1-(cyclopropylmethyl)-1H-indazol-3-amine;
1-(4-amino-6-methyl-1,3,5-triazin-2-yl)-N-(2,3-dihydro-1H-inden-1-yl)-1H-benzimidazol-2-amine;
3-((1-(4-amino-6-methyl-1,3,5-triazin-2-yl)-5,6-dichloro-1H-benzimidazol-2-yl)amino)phenol;
5-((1-(4-amino-6-methyl-1,3,5-triazin-2-yl)-1H-benzimidazol-2-yl)amino)-1H-pyrazole-4-carbonitrile;
1-(4-amino-6-methyl-1,3,5-triazin-2-yl)-N-(1-methyl-1H-pyrazol-5-yl)-1H-benzimidazol-2-amine;
ethyl 5-((1-(4-amino-6-methyl-1,3,5-triazin-2-yl)-1H-benzimidazol-2-yl)amino)-1H-pyrazole-4-carboxylate;
methyl 3-((1-(4-amino-6-methyl-1,3,5-triazin-2-yl)-1H-benzimidazol-2-yl)amino)benzoate;
4-(2-((3-amino-1H-pyrazol-5-yl)oxy)-1H-benzimidazol-1-yl)-6-methyl-1,3,5-triazin-2-amine;
4-(2-(3-methoxyphenoxy)-1H-benzimidazol-1-yl)-6-methyl-1,3,5-triazin-2-amine;
1-(4-amino-6-methyl-1,3,5-triazin-2-yl)-N-1H-imidazol-2-yl-1H-benzimidazol-2-amine;
4-((1-(4-amino-6-methyl-1,3,5-triazin-2-yl)-1H-benzimidazol-2-yl)amino)-2,3-dihydro-1H-isoindol-1-one;
1-(4-amino-6-methyl-1,3,5-triazin-2-yl)-N-1,3-thiazol-2-yl-1H-benzimidazol-2-amine;
1-(4-((1-(4-amino-6-methyl-1,3,5-triazin-2-yl)-1H-benzimidazol-2-yl)amino)-2-hydroxyphenyl)ethanone;
methyl 1-((1-(4-amino-6-methyl-1,3,5-triazin-2-yl)-1H-benzimidazol-2-yl)amino)cyclopropanecarboxylate;
1-(4-amino-6-methyl-1,3,5-triazin-2-yl)-N-(3-tert-butyl-1H-pyrazol-5-yl)-1H-benzimidazol-2-amine;
N-(1-(4-amino-6-methyl-1,3,5-triazin-2-yl)-1H-benzimidazol-2-yl)-5-(trifluoromethyl)-1H-indazol-3-amine;
1-(4-amino-6-methyl-1,3,5-triazin-2-yl)-N-(1-(4-chlorophenyl)cyclopropyl)-1H-benzimidazol-2-amine;
methyl 1-(4-amino-6-methyl-1,3,5-triazin-2-yl)-2-((3-methoxyphenyl)amino)-1H-benzimidazole-6-carboxylate;
1-(4-amino-6-methyl-1,3,5-triazin-2-yl)-N-(3-cyclohexyl-1H-pyrazol-5-yl)-1H-benzimidazol-2-amine;
3-((1-(6-amino-2-methyl-4-pyrimidinyl)-1H-benzimidazol-2-yl)amino)phenol;
3-((1-(6-pyrimidin-5-ylamino-2-methyl-4-pyrimidinyl)-1H-benzimidazol-2-yl)amino)phenol
1-(6-amino-2-methyl-4-pyrimidinyl)-N-1H-pyrazol-3-yl-1H-benzimidazol-2-amine;
3-((1-(6-pyrimidin-2-ylamino-2-methyl-4-pyrimidinyl)-1H-benzimidazol-2-yl)amino)phenol
1-(6-amino-2-methyl-4-pyrimidinyl)-N-(3-cyclopropyl-1H-pyrazol-5-yl)-1H-benzimidazol-2-amine;
3-((1-(2-amino-6-methyl-4-pyrimidinyl)-1H-benzimidazol-2-yl)oxy)phenol;
4-(2-(3-methoxyphenoxy)-1H-benzimidazol-1-yl)-6-methyl-2-pyrimidinamine;
1-(4-amino-6-methyl-1,3,5-triazin-2-yl)-5,6-dichloro-N-1H-pyrazol-5-yl-1H-benzimidazol-2-amine;
1-(4-((1-(4-amino-6-methyl-1,3,5-triazin-2-yl)-1H-benzimidazol-2-yl)amino)phenyl)-3-(4-methoxyphenyl)thiourea;
1-(4-((1-(4-amino-6-methyl-1,3,5-triazin-2-yl)-1H-benzimidazol-2-yl)amino)phenyl)-3-(3-methoxyphenyl)thiourea;
1-(4-amino-6-methyl-1,3,5-triazin-2-yl)-4-methyl-N-1H-pyrazol-5-yl-1H-benzimidazol-2-amine;
N-(4-((1-(4-amino-6-methyl-1,3,5-triazin-2-yl)-1H-benzimidazol-2-yl)amino)phenyl)-2,6-dichlorobenzenesulfonamide;
((4-methyl-6-(2-(1H-pyrazol-3-ylamino)-1H-benzimidazol-1-yl)-1,3,5-triazin-2-yl)amino)acetonitrile;
N-3-isoxazolyl-1-(4-methyl-6-(methylamino)-1,3,5-triazin-2-yl)-1H-benzimidazol-2-amine;

((4-(2-(3-isoxazolylamino)-1H-benzimidazol-1-yl)-6-methyl-1,3,5-triazin-2-yl)amino)acetonitrile;
1-(4-((1-(4-amino-6-methyl-1,3,5-triazin-2-yl)-1H-benzimidazol-2-yl)amino)phenyl)-3-ethylthiourea;
1-(4-((1-(4-amino-6-methyl-1,3,5-triazin-2-yl)-1H-benzimidazol-2-yl)amino)phenyl)-3-(3,5-dichlorophenyl)thiourea;
N-(4-((1-(4-amino-6-methyl-1,3,5-triazin-2-yl)-1H-benzimidazol-2-yl)amino)phenyl)-2-(trifluoromethyl)benzenesulfonamide;
1-(4-((1-(4-((cyanomethyl)amino)-6-methyl-1,3,5-triazin-2-yl)-1H-benzimidazol-2-yl)amino)phenyl)-3-methylurea;
1-(4-((1-(4-((cyanomethyl)amino)-6-methyl-1,3,5-triazin-2-yl)-1H-benzimidazol-2-yl)amino)phenyl)-3-ethylurea;
1-(4-methyl-6-(methylamino)-1,3,5-triazin-2-yl)-N-1H-pyrazol-3-yl-6-(5-pyrimidinyl)-1H-benzimidazol-2-amine;
1-(4-((1-(4-amino-6-methyl-1,3,5-triazin-2-yl)-1H-benzimidazol-2-yl)amino)phenyl)-3-(4-pyridinyl)urea;
mixture of 1-(4-methyl-6-(methylamino)-1,3,5-triazin-2-yl)-5-(2-methyl-4-pyridinyl)-N-1H-pyrazol-3-yl-1H-benzimidazol-2-amine and 1-(4-methyl-6-(methylamino)-1,3,5-triazin-2-yl)-6-(2-methyl-4-pyridinyl)-N-1H-pyrazol-3-yl-1H-benzimidazol-2-amine;
mixture of 1-(4-amino-6-methyl-1,3,5-triazin-2-yl)-5-(1H-pyrazol-4-yl)-N-1H-pyrazol-5-yl-1H-benzimidazol-2-amine and 1-(4-amino-6-methyl-1,3,5-triazin-2-yl)-6-(1H-pyrazol-4-yl)-N-1H-pyrazol-5-yl-1H-benzimidazol-2-amine;
mixture of 1-(4-methyl-6-(methylamino)-1,3,5-triazin-2-yl)-N-1H-pyrazol-3-yl-5-(5-pyrimidinyl)-1H-benzimidazol-2-amine and 1-(4-methyl-6-(methylamino)-1,3,5-triazin-2-yl)-N-1H-pyrazol-3-yl-6-(5-pyrimidinyl)-1H-benzimidazol-2-amine;
N-(4-((1-(4-methyl-6-(methylamino)-1,3,5-triazin-2-yl)-1H-benzimidazol-2-yl)amino)phenyl)acetamide;
mixture of 1-(4-amino-6-methyl-1,3,5-triazin-2-yl)-N-1H-pyrazol-5-yl-5-(4-pyridinyl)-1H-benzimidazol-2-amine; and 1-(4-amino-6-methyl-1,3,5-triazin-2-yl)-N-1H-pyrazol-5-yl-6-(4-pyridinyl)-1H-benzimidazol-2-amine;
1-(4-amino-6-methyl-1,3,5-triazin-2-yl)-N-1H-pyrazol-5-yl-6-(3-pyridinyl)-1H-benzimidazol-2-amine;
mixture of 1-(4-methyl-6-(methylamino)-1,3,5-triazin-2-yl)-N-1H-pyrazol-3-yl-5-(1,2,3,6-tetrahydro-4-pyridinyl)-1H-benzimidazol-2-amine; and 1-(4-methyl-6-(methylamino)-1,3,5-triazin-2-yl)-N-1H-pyrazol-3-yl-6-(1,2,3,6-tetrahydro-4-pyridinyl)-1H-benzimidazol-2-amine;
1-(4-amino-6-methyl-1,3,5-triazin-2-yl)-N-1H-pyrazol-5-yl-6-(4-pyridinyl)-1H-benzimidazol-2-amine;
mixture of (5-(1-(4-amino-6-methyl-1,3,5-triazin-2-yl)-2-(1H-pyrazol-5-ylamino)-1H-benzimidazol-6-yl)-2-methoxyphenyl)methanol; and (5-(1-(4-amino-6-methyl-1,3,5-triazin-2-yl)-2-(1H-pyrazol-5-ylamino)-1H-benzimidazol-5-yl)-2-methoxyphenyl)methanol;
mixture of N-methyl-4-(1-(4-methyl-6-(methylamino)-1,3,5-triazin-2-yl)-2-(1H-pyrazol-3-ylamino)-1H-benzimidazol-5-yl)benzamide; and N-methyl-4-(1-(4-methyl-6-(methylamino)-1,3,5-triazin-2-yl)-2-(1H-pyrazol-3-ylamino)-1H-benzimidazol-6-yl)benzamide;
mixture of 2-fluoro-N-methyl-4-(1-(4-methyl-6-(methylamino)-1,3,5-triazin-2-yl)-2-(1H-pyrazol-3-ylamino)-1H-benzimidazol-5-yl)benzamide; and 2-fluoro-N-methyl-4-(1-(4-methyl-6-(methylamino)-1,3,5-triazin-2-yl)-2-(1H-pyrazol-3-ylamino)-1H-benzimidazol-6-yl)benzamide;
N-(4-((1-(4-((cyanomethyl)amino)-6-methyl-1,3,5-triazin-2-yl)-1H-benzimidazol-2-yl)amino)phenyl)acetamide;
mixture of ((4-(5-(3-(hydroxymethyl)-4-methoxyphenyl)-2-(1H-pyrazol-3-ylamino)-1H-benzimidazol-1-yl)-6-methyl-1,3,5-triazin-2-yl)amino)acetonitrile; and ((4-(6-(3-(hydroxymethyl)-4-methoxyphenyl)-2-(1H-pyrazol-3-ylamino)-1H-benzimidazol-1-yl)-6-methyl-1,3,5-triazin-2-yl)amino)acetonitrile;
mixture of N-(4-(1-(4-methyl-6-(methylamino)-1,3,5-triazin-2-yl)-2-(1H-pyrazol-3-ylamino)-1H-benzimidazol-5-yl)phenyl)acetamide; and N-(4-(1-(4-methyl-6-(methylamino)-1,3,5-triazin-2-yl)-2-(1H-pyrazol-3-ylamino)-1H-benzimidazol-6-yl)phenyl)acetamide;
mixture of 1-(4-methyl-6-(methylamino)-1,3,5-triazin-2-yl)-N-1H-pyrazol-3-yl-5-(3-pyridinyl)-1H-benzimidazol-2-amine; and 1-(4-methyl-6-(methylamino)-1,3,5-triazin-2-yl)-N-1H-pyrazol-3-yl-6-(3-pyridinyl)-1H-benzimidazol-2-amine;
mixture of 1-(4-amino-6-methyl-1,3,5-triazin-2-yl)-2-(1H-pyrazol-5-ylamino)-1H-benzimidazol-5-ol; and -(4-amino-6-methyl-1,3,5-triazin-2-yl)-2-(1H-pyrazol-5-ylamino)-1H-benzimidazol-6-ol;
mixture of ((4-methyl-6-(2-(1H-pyrazol-3-ylamino)-5-(4-pyridinyl)-1H-benzimidazol-1-yl)-1,3,5-triazin-2-yl)amino)acetonitrile; and ((4-methyl-6-(2-(1H-pyrazol-3-ylamino)-6-(4-pyridinyl)-1H-benzimidazol-1-yl)-1,3,5-triazin-2-yl)amino)acetonitrile;
mixture of 1-(4-amino-6-methyl-1,3,5-triazin-2-yl)-5-methoxy-N-1H-pyrazol-5-yl-1H-benzimidazol-2-amine; and 1-(4-amino-6-methyl-1,3,5-triazin-2-yl)-6-methoxy-N-1H-pyrazol-5-yl-1H-benzimidazol-2-amine;
mixture of (2-methoxy-5-(1-(4-methyl-6-(methylamino)-1,3,5-triazin-2-yl)-2-(1H-pyrazol-3-ylamino)-1H-benzimidazol-5-yl)phenyl)methanol; and (2-methoxy-5-(1-(4-methyl-6-(methylamino)-1,3,5-triazin-2-yl)-2-(1H-pyrazol-3-ylamino)-1H-benzimidazol-6-yl)phenyl)methanol;
N˜5˜-(1-(4-amino-6-methyl-1,3,5-triazin-2-yl)-1H-benzimidazol-2-yl)-2,5-pyrimidinediamine;
((4-(5,6-dimethyl-2-(1H-pyrazol-3-ylamino)-1H-benzimidazol-1-yl)-6-methyl-1,3,5-triazin-2-yl)amino)acetonitrile;
4-((1-(4-amino-6-methyl-1,3,5-triazin-2-yl)-1H-benzimidazol-2-yl)amino)-N-methylbenzamide;
5,6-dimethyl-1-(4-methyl-6-(methylamino)-1,3,5-triazin-2-yl)-N-1H-pyrazol-3-yl-1H-benzimidazol-2-amine;
mixture of 1-(4-methyl-6-(methylamino)-1,3,5-triazin-2-yl)-N-1H-pyrazol-3-yl-5-(4-pyridinyl)-1H-benzimidazol-2-amine; and 1-(4-methyl-6-(methylamino)-1,3,5-triazin-2-yl)-N-1H-pyrazol-3-yl-6-(4-pyridinyl)-1H-benzimidazol-2-amine;
mixture of (5-(1-(4-amino-6-methyl-1,3,5-triazin-2-yl)-2-(1H-pyrazol-5-ylamino)-1H-benzimidazol-5-yl)-2-methoxyphenyl)methanol;
N-(4-((1-(4-amino-6-methyl-1,3,5-triazin-2-yl)-1H-benzimidazol-2-yl)amino)phenyl)cyclopropanecarboxamide;
5-(4-methoxyphenyl)-1-(4-methyl-6-(methylamino)-1,3,5-triazin-2-yl)-N-1H-pyrazol-3-yl-1H-benzimidazol-2-amine;
3-((4-methyl-6-(2-(1H-pyrazol-3-ylamino)-1H-benzimidazol-1-yl)-1,3,5-triazin-2-yl)amino)propanenitrile;
2-((4-methyl-6-(2-(1H-pyrazol-3-ylamino)-1H-benzimidazol-1-yl)-1,3,5-triazin-2-yl)amino)ethanol;
5,6-difluoro-1-(4-methyl-6-(methylamino)-1,3,5-triazin-2-yl)-N-1H-pyrazol-5-yl-1H-benzimidazol-2-amine;
1-(4-amino-6-methyl-1,3,5-triazin-2-yl)-N-1H-pyrazol-5-yl-5-(4-pyridinyl)-1H-benzimidazol-2-amine;
mixture of 5-(4-fluorophenyl)-1-(4-methyl-6-(methylamino)-1,3,5-triazin-2-yl)-N-1H-pyrazol-3-yl-1H-benzimidazol-2-amine; and 6-(4-fluorophenyl)-1-(4-methyl-6-(methylamino)-1,3,5-triazin-2-yl)-N-1H-pyrazol-3-yl-1H-benzimidazol-2-amine;
4-((1-(4-amino-6-methyl-1,3,5-triazin-2-yl)-1H-benzimidazol-2-yl)amino)-2-fluorophenol;
5-((1-(4-amino-6-methyl-1,3,5-triazin-2-yl)-1H-benzimidazol-2-yl)amino)-1,3-dihydro-2H-benzimidazol-2-one;
1-(4-methyl-6-(methylamino)-1,3,5-triazin-2-yl)-N-1H-pyrazol-3-yl-5-(5-pyrimidinyl)-1H-benzimidazol-2-amine;
1-(4-((2-methoxyethyl)amino)-6-methyl-1,3,5-triazin-2-yl)-N-1H-pyrazol-3-yl-1H-benzimidazol-2-amine;
1-(4-(cyclopropylamino)-6-methyl-1,3,5-triazin-2-yl)-N-1H-pyrazol-5-yl-1H-benzimidazol-2-amine;
1-(4-amino-6-methyl-1,3,5-triazin-2-yl)-N-(6-methoxy-3-pyridinyl)-1H-benzimidazol-2-amine;
4-((1-(4-amino-6-methyl-1,3,5-triazin-2-yl)-1H-benzimidazol-2-yl)amino)-N-ethylbenzamide;
1-(4-(benzylamino)-6-methyl-1,3,5-triazin-2-yl)-N-1H-pyrazol-3-yl-1H-benzimidazol-2-amine;
N-(1-(4-amino-6-methyl-1,3,5-triazin-2-yl)-1H-benzimidazol-2-yl)-1H-indazol-4-amine;
1-(4-methyl-6-((2-(1-piperazinyl)ethyl)amino)-1,3,5-triazin-2-yl)-N-1H-pyrazol-3-yl-1H-benzimidazol-2-amine;
1-(4-methyl-6-((2-(4-morpholinyl)ethyl)amino)-1,3,5-triazin-2-yl)-N-1H-pyrazol-3-yl-1H-benzimidazol-2-amine;
4-((1-(4-amino-6-methyl-1,3,5-triazin-2-yl)-1H-benzimidazol-2-yl)amino)-N-4-pyridinylbenzamide;
1-(4-(ethylamino)-6-methyl-1,3,5-triazin-2-yl)-N-1H-pyrazol-3-yl-1H-benzimidazol-2-amine;
N-(3-((1-(4-amino-6-methyl-1,3,5-triazin-2-yl)-1H-benzimidazol-2-yl)amino)phenyl)acetamide;
1-(4-methyl-6-((tetrahydro-2H-pyran-4-ylmethyl)amino)-1,3,5-triazin-2-yl)-N-1H-pyrazol-3-yl-1H-benzimidazol-2-amine;
1-(4-methyl-6-(tetrahydro-2H-pyran-4-ylamino)-1,3,5-triazin-2-yl)-N-1H-pyrazol-3-yl-1H-benzimidazol-2-amine;
N-(4-((1-(4-amino-6-methyl-1,3,5-triazin-2-yl)-1H-benzimidazol-2-yl)amino)phenyl)-2,3-dihydro-1-benzofuran-5-carboxamide;
1-(4-methyl-6-((1-methylethyl)amino)-1,3,5-triazin-2-yl)-N-1H-pyrazol-3-yl-1H-benzimidazol-2-amine;
1-(4-((1H-imidazol-2-ylmethyl)amino)-6-methyl-1,3,5-triazin-2-yl)-N-1H-pyrazol-3-yl-1H-benzimidazol-2-amine;
N-(1-(4-amino-6-methyl-1,3,5-triazin-2-yl)-1H-benzimidazol-2-yl)-1,4-benzenediamine;
N-(4-((1-(4-amino-6-methyl-1,3,5-triazin-2-yl)-1H-benzimidazol-2-yl)amino)phenyl)-3-(dimethylamino)benzamide;
1-(4-amino-6-methyl-1,3,5-triazin-2-yl)-N-(1-methyl-1H-pyrazol-4-yl)-1H-benzimidazol-2-amine;
N-(4-((1-(4-amino-6-methyl-1,3,5-triazin-2-yl)-1H-benzimidazol-2-yl)amino)phenyl)cyclopentanecarboxamide;
N-(4-((1-(4-amino-6-methyl-1,3,5-triazin-2-yl)-1H-benzimidazol-2-yl)amino)phenyl)-2,2-dimethylpropanamide;
1-(4-((cyclopropylmethyl)amino)-6-methyl-1,3,5-triazin-2-yl)-N-1H-pyrazol-3-yl-1H-benzimidazol-2-amine;
1-(4-amino-6-methyl-1,3,5-triazin-2-yl)-N-2-pyrimidinyl-1H-benzimidazol-2-amine;
1-(4-methyl-1,3,5-triazin-2-yl)-N-1H-pyrazol-3-yl-1H-benzimidazol-2-amine;
N-(4-((1-(4-amino-6-methyl-1,3,5-triazin-2-yl)-1H-benzimidazol-2-yl)amino)phenyl)-2,1,3-benzothiadiazole-4-carboxamide;
1-(4-methyl-6-(2-pyrimidinylamino)-1,3,5-triazin-2-yl)-N-1H-pyrazol-3-yl-1H-benzimidazol-2-amine;
4-((1-(4-amino-6-methyl-1,3,5-triazin-2-yl)-1H-benzimidazol-2-yl)amino)-N-(1-methylethyl)benzamide;
1-(4-amino-6-methyl-1,3,5-triazin-2-yl)-N-(3-methylphenyl)-1H-benzimidazol-2-amine;
1-(4-amino-6-methyl-1,3,5-triazin-2-yl)-N-(1-(2,2,2-trifluoroethyl)-1H-pyrazol-3-yl)-1H-benzimidazol-2-amine;
1-(4-amino-6-methyl-1,3,5-triazin-2-yl)-N-4-pyrimidinyl-1H-benzimidazol-2-amine;
5-((1-(4-amino-6-methyl-1,3,5-triazin-2-yl)-1H-benzimidazol-2-yl)amino)-N-methyl-1H-pyrazole-3-carboxamide;
1-(4-((1-(4-((cyanomethyl)amino)-6-methyl-1,3,5-triazin-2-yl)-1H-benzimidazol-2-yl)amino)phenyl)-3-(3,5-dichlorophenyl)thiourea;
1-(4-amino-6-methyl-1,3,5-triazin-2-yl)-N-2-pyrazinyl-1H-benzimidazol-2-amine;
3-((1-(4-amino-6-methyl-1,3,5-triazin-2-yl)-1H-benzimidazol-2-yl)amino)benzonitrile;
1-(4-amino-6-methyl-1,3,5-triazin-2-yl)-N-4-pyridazinyl-1H-benzimidazol-2-amine;
4-((1-(4-amino-6-methyl-1,3,5-triazin-2-yl)-1H-benzimidazol-2-yl)amino)-2-pyrrolidinone;
5-((1-(4-amino-6-methyl-1,3,5-triazin-2-yl)-1H-benzimidazol-2-yl)amino)-N-cyclopropyl-1H-pyrazole-3-carboxamide;
1-(4-amino-6-methyl-1,3,5-triazin-2-yl)-N-cyclopentyl-1H-benzimidazol-2-amine
1-(4-amino-6-methyl-1,3,5-triazin-2-yl)-N-3-pyridazinyl-1H-benzimidazol-2-amine;
1-(4-amino-6-methyl-1,3,5-triazin-2-yl)-N-cyclohexyl-1H-benzimidazol-2-amine;
3-((1-(4-amino-6-methyl-1,3,5-triazin-2-yl)-1H-benzimidazol-2-yl)amino)-2-pyrrolidinone;
1-(4-amino-6-methyl-1,3,5-triazin-2-yl)-N-3-pyrrolidinyl-1H-benzimidazol-2-amine;
1-(4-(dimethylamino)-6-methyl-1,3,5-triazin-2-yl)-N-1H-pyrazol-5-yl-1H-benzimidazol-2-amine;
N-(4-((1-(4-amino-6-methyl-1,3,5-triazin-2-yl)-1H-benzimidazol-2-yl)amino)phenyl)-4-tert-butylbenzamide
N-(4-((1-(4-amino-6-methyl-1,3,5-triazin-2-yl)-1H-benzimidazol-2-yl)amino)phenyl)cyclohexanecarboxamide;
N-(4-((1-(4-amino-6-methyl-1,3,5-triazin-2-yl)-1H-benzimidazol-2-yl)amino)phenyl)-3-(trifluoromethyl)benzamide;
N-(4-((1-(4-amino-6-methyl-1,3,5-triazin-2-yl)-1H-benzimidazol-2-yl)amino)phenyl)-4-(trifluoromethoxy)benzamide;
ethyl 5-((1-(4-amino-6-methyl-1,3,5-triazin-2-yl)-1H-benzimidazol-2-yl)amino)-1H-pyrazole-3-carboxylate;
N-(4-((1-(4-amino-6-methyl-1,3,5-triazin-2-yl)-1H-benzimidazol-2-yl)amino)phenyl)-4-fluorobenzenesulfonamide;
1-(4-amino-6-methyl-1,3,5-triazin-2-yl)-N-(1-(4-morpholinylcarbonyl)-1H-pyrazol-4-yl)-1H-benzimidazol-2-amine;
N-(1-(4-amino-6-methyl-1,3,5-triazin-2-yl)-1H-benzimidazol-2-yl)-1,3-benzoxazol-6-amine; and
4-((1-(4-amino-6-methyl-1,3,5-triazin-2-yl)-1H-benzimidazol-2-yl)amino)-N-4-pyridinyl-1H-pyrazole-1-carboxamide.

### Embodiments

A. In one embodiment provided are compounds of Formula (I) as defined in Summary.
B. In another embodiment provided are compounds of Formula (I):

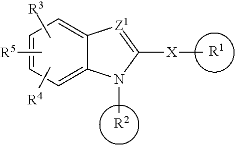

where:

Z¹ is —N— or —CH—;

X is —NR⁶— or —O— where R⁶ is hydrogen or alkyl;

R¹ is aryl, heteroaryl, cycloalkyl, fused cycloalkenyl, or heterocyclyl; each ring substituted with R^{a}, R^{b}, or R^{c} independently selected from hydrogen, alkyl, alkylthio, alkoxy, hydroxy, alkoxycarbonyl, carboxy, halo, haloalkyl, haloalkoxy, aminocarbonyl, aminosulfonyl, cycloalkyl, cycloalkylalkyl, acyl, cyano, aminoalkyl, hydroxyalkyl, optionally substituted heteroaryl, optionally substituted phenyl, amino, ureido, thioureido, monosubstituted, or disubstituted amino;

R² is:

(i)

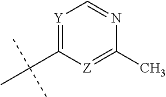

where Y and Z are independently —N═ or —C═; or

(ii) a five or six membered heterocyclyl ring; each ring substituted with R^{d} and R^{e} where R^{d} and R^{e} are independently hydrogen, alkyl, alkoxy, halo, haloalkyl, haloalkoxy, amino, monosubstituted amino or disubstituted amino;

R³ and R⁴ are independently hydrogen, alkyl, halo, alkoxy, haloalkyl, hydroxyalkyl, alkoxyalkyl, cyano, carboxy, alkoxycarbonyl, aryl, heteroaryl, heterocyclyl, aralkyl, heteroaralkyl, heterocyclylalkyl, amino, monosubstituted amino, disubstituted amino, sulfonyl, acyl, hydroxyalkyloxy, alkoxyalkyloxy, aminoalkyl, aminoalkoxy, aryloxy, heteroaryloxy, heterocyclyloxy, aralkoxy, heteroaralkoxy, heterocyclylalkyloxy, aminosulfonyl, aminocarbonyl, or acylamino where the aromatic or alicyclic ring in R³ and R⁴ is substituted with R^{f}, R^{g} or R^{h} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; and

R⁵ is hydrogen, alkyl, halo, alkoxy, haloalkyl, hydroxyalkyl, alkoxyalkyl, cyano, carboxy, alkoxycarbonyl, amino, alkylamino, or dialkylamino; or

a pharmaceutically acceptable salt thereof;

where the following terms within the scope of (B) either alone or as part of another term have the definitions given below:

“Aminocarbonyl” means a —CONRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl;

“Aminosulfonyl” means a —SO₂NRR′ radical where R is independently hydrogen, alkyl, hydroxyalkyl, alkoxyalkyl, or aminoalkyl, each as defined herein and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl;

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl;

“Acylamino” means a —NHCOR radical where R is alkyl, haloalkyl, cycloalkyl, cycloalkylalkyl, amino, monosubstituted or disubstituted amino, aryl, aralkyl, heteroaryl, heteroaralkyl, heterocyclyl, or heterocyclylalkyl;

“Disubstituted amino” means an —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl;

“Monosubstituted amino” means an —NHR radical where R is alkyl, cyanoalkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl;

“Optional substituted phenyl” means phenyl ring that is optionally substituted with one, two, or three substitutents independently selected from alkyl, halo, haloalkyl, hydroxyl, haloalkoxy, alkoxy, amino, alkylamino, cyano, or dialkylamino;

“Optional substituted heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon, that is optionally substituted with one, two, or three substitutents independently selected from alkyl, halo, haloalkyl, haloalkoxy, alkoxy, amino, alkylamino, cyano, or dialkylamino;

“Sulfonyl” means a —SO₂R radical where R is alkyl, haloalkyl, aryl, aralkyl, heteroaryl, heteroaralkyl, heterocyclyl, or heterocyclylalkyl;

“Heterocyclyl” means a saturated or unsaturated monovalent monocyclic group of 5 to 8 ring atoms in which one or two ring atoms are heteroatom selected from N, O, or S(O)ₙ, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this Application as “bicyclic heterocyclyl” ring and is a subset of fused heterocyclyl. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

and other groups are as defined in the Definitions section.

I. Within embodiments A and B in one embodiment, the compound of Formula (I) is where:

Z¹ is —N— and X is —NR⁶—. Preferably R⁶ is alkyl, more preferably methyl.

II. Within embodiments A and B, in another embodiment, the compound of Formula (I) is where:

Z¹ is —N— and X is —O—.

III. Within embodiments A and B, in yet another embodiment, the compound of Formula (I) is where:

Z¹ is —CH— and X is —NR⁶—. Preferably R⁶ is alkyl, more preferably methyl.

IV. Within embodiments A and B, in yet another embodiment, the compound of Formula (I) is where:

Z¹ is —CH— and X is —O—.

V. Within embodiments A and B, in yet another embodiment, the compound of Formula (I) is where:

Z¹ is —N— and X is —NH—.

(a) With the above embodiments A, B, (I), (II), (III), (IV) and (V), in one group of compounds R¹ is phenyl substituted as defined in the Summary of the Invention.
(b) With the above embodiments A, B, (I), (II), (III), (IV) and (V), in another group of compounds R¹ is heteroaryl, preferably pyrazolyl, substituted as defined in the Summary of the Invention.
(c) With the above embodiments A, B, (I), (II), (III), (IV) and (V), in another group of compounds R¹ is cycloalkyl substituted as defined in the Summary of the Invention.
(d) With the above embodiments A, B, (I), (II), (III), (IV) and (V), in another group of compounds R¹ is heterocyclyl substituted as defined in the Summary of the Invention.
(i) With the above embodiments A, B, (I), (II), (III), (IV), (V), (a), (b), (c) and (d), and groups contained therein, in one group of compound, R² is

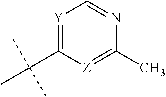

where Y and Z are independently —N═ or —C═; preferably Z is nitrogen, more preferably both Y and Z are nitrogen, and is substituted with R^{d} where R^{d} is hydrogen, alkyl, alkoxy, halo, haloalkyl, haloalkoxy, amino, monosubstituted amino or disubstituted amino, preferably R^{d} is amino or monosubstituted amino and is located at the 4-position of the triazin-2-yl ring and R^{e} is hydrogen. Preferably, R² is 4-amino-6-methyl-1,3,5-triazin-2-yl.
(ii) With the above embodiments A, B, (I), (II), (III), (IV), (V), (a), (b), (c) and (d), and groups contained therein, in one group of compound, R² is

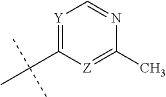

where Y and Z are independently —N═ or —C═; preferably Z is nitrogen, more preferably both Y and Z are nitrogen and is substituted with R^{d} where R^{d} is hydrogen, alkyl, alkoxy, halo, haloalkyl, haloalkoxy, amino, monosubstituted amino or disubstituted amino. Preferably R² is 4-cyanomethylamino-6-methyl-1,3,5-triazin-2-yl.
(1) With the above embodiments A, B, (I), (II), (III), (IV), (V), (a), (b), (c) and (d), (i) and (ii) and groups contained therein, in one group of compound, R¹ is phenyl substituted with R^{a}, R^{b} or R^{c} where R^{a} is hydrogen, R^{b} is hydrogen or hydroxy, and R^{c} is hydrogen, cyano, acyl, ureido, thioureido, alkoxycarbonyl, alkoxy, hydroxy, amino, cycloalkyl, carboxy, halo, aminocarbonyl, aminosulfonyl, alkyl, or monosubstituted amino (—NRR′ where R is hydrogen and R′ is hydrogen acyl, or sulfonyl). Preferably, R¹ is 3,5-dihydroxyphenyl; 3-(—NHCONHCH₃)phenyl; 3-(—NHCONHCH₂CH₃)-phenyl; 3-(3-hydroxyphenyl-NHCONH—) phenyl; 3-hydroxy-5-methoxycarbonyl-phenyl; 3-hydroxy-4-methoxyphenyl; 3-(3-methoxyphenyl-NHCONH—)phenyl; 3-(4-methoxycarbonylphenyl-NHCONH—)phenyl; 4-carboxy-2-hydroxyphenyl; 4-hydroxyphenyl; 3-carboxyphenyl; 4-fluoro-3-hydroxyphenyl; 3-methoxyphenyl; 3-CONH₂-phenyl; 3-hydroxy-4-methylphenyl; 4-(4-fluorophenylsulfonyl)-aminophenyl; 4-(4-methylphenylsulfonyl)-aminophenyl; 3-SO₂NH₂-phenyl; phenyl; 4-methylsulfonylaminophenyl; 3-hydroxy-4-methoxycarbonylphenyl; 4-ethylcarbonylaminophenyl; 3-methylphenyl; 3-chlorophenyl; 3-fluorophenyl; 3-cyanophenyl; 4-acetylaminophenyl; 3-hydroxyphenyl; 3-methoxycarbonylphenyl; 3-hydroxy-4-acetylphenyl; 4-[4-methoxyphenylNHCSNH—]phenyl; 4-[3-methoxyphenylNHCSNH—]phenyl; 2,6-dichlorophenylsulfonyl-aminophenyl; 4-[ethylNHCSNH—]phenyl; 3-[3,5-dichlorophenylNHCSNH—]phenyl; 4-[2-trifluoromethylphenyl-sulfonylamino]]phenyl; 4-[—NHCONHCH₃]phenyl; 4-[—NHCONHCH₂CH₃]phenyl; 4-[—NHCONHpyridin-4-yl]phenyl; 4-methylcarbonylaminophenyl; 4-cyclopropylcarbonylamino-phenyl; 3-F-4-hydroxyphenyl; 4-(ethylaminocarbonyl)phenyl; 4-(pyridin-4-ylaminocarbonyl)-phenyl; 3-methylcarbonylamino-phenyl; 4-(2,3-dihydro-1-benzofuran-5-ylcarbonylamino)phenyl; 4-aminophenyl; 4-(3-dimethylaminophenyl-carbonylamino)phenyl; 4-(cyclopentylcarbonylamino)-phenyl; 4-(tert-butylcarbonylamino)-phenyl; 4-(2.1.3-benzothiadiazol-4-ylcarbonylamino)phenyl; 4-(2-isopropylaminocarbonyl)-phenyl; 4-(3,54-diClphenylNHCSNH—)phenyl; 4-(4-tert-butylphenylcarbonyl-amino)phenyl; 4-(cyclohexylcarbonyl-amino)phenyl; 4-(3-CF₃-phenylcarbonyl-amino)phenyl; 4-(4-OCF₃phenylcarbonyl-amino)phenyl; or 4-(4-fluorophenylsulfonyl-amino)phenyl.
(2) With the above embodiments A, B, (I), (II), (III), (IV), (V), (VI), (VII), (a), (b), (c) and (d), (i) and (ii) and groups contained therein, in one group of compound, in one group of compounds R¹ is heteroaryl, preferably pyrazolyl, indazolyl, indolyl, pyridinyl, isothiazolyl, 1.2.4-triazolyl, azaindazolyl, imidazolyl, thiazolyl, isoxazolyl, pyrimidinyl, pyrazinyl, pyridazinyl, or benzoxazolyl, preferably pyrazol-3-yl, substituted with R^{a}, R^{b} or R^{c} where R^{a} and R^{b} are hydrogen, and R^{c} is hydrogen, cyano, acyl, ureido, thioureido, alkoxycarbonyl, alkoxy, hydroxy, amino, cycloalkyl, cycloalkylalkyl, optionally substituted heteroaryl, carboxy, halo, aminocarbonyl, alkyl, haloalkyl, or monosubstituted amino (—NRR′ where R is hydrogen and R′ is hydrogen acyl, or sulfonyl). Preferably, R^{c} is hydrogen, cyano, acyl, alkoxycarbonyl, alkoxy, hydroxy, amino, cycloalkyl, cycloalkylalkyl, optionally substituted heteroaryl, halo, aminocarbonyl, alkyl, or haloalkyl. Preferably R¹ is pyrazol-5-yl; pyrazol-3-yl; indazol-6-yl; indol-6-yl; 3-cyclopropylpyrazol-5-yl; 2-aminopyridin-5-yl; indazol-3-yl; indol-4-yl; 4-fluoroindazol-3-yl; pyridin-3-yl; pyrazol-4-yl; 6-aminopyridin-2-yl; 1-methylpyrazol-3-yl; 3-cyclopropylpyrazol-5-yl; 3-methylisothiazol-5-yl; 5-methylpyrazol-3-yl; 3-furan-2-ylpyrazol-5-yl; 3-methylpyrazol-5-yl; 3-isopropylpyrazol-5-yl; 3-thiophen-2-ylpyrazol-5-yl; 1.2.4-triazol-3-yl; 5-hydroxypyridin-3-yl; 5-methyl-1.2.4-triazol-3-yl; 7-azaindazol-3-yl; 1-methylindazol-3-yl; 2-hydroxypyridin-5-yl; 3-hydroxypyrazol-5-yl; 1-cyclopropylmethyl-indazol-3-yl; 4-cyanopyrazol-5-yl; 1-methylpyrazol-5-yl; 4-ethoxycarbonylpyrazol-5-yl; 3-aminopyrazol-5-yl; imidazol-2-yl; 1,3-thiazol-2-yl; 3-tert-butylpyrazol-5-yl; 5-trifluoromethylindazol-3-yl; 3-cyclohexylpyrazol-5-yl; isoxazol-3-yl; 2-aminopyrimidin-4-yl; 6-methoxypyridin-3-yl; 1H-indazol-4-yl; 1-methylpyrazol-4-yl; pyrimidin-2-yl; 1-(2,2,2-trifluoroethyl)pyrazol-3-ylpyrimidin-4-yl; 3-(methylaminocarbonyl)-pyrazol-5-yl; pyrazin-2-yl; pyridazin-4-yl; 3-(cyclopropylamino-carbonyl)-pyrazol-5-yl; pyridazin-3-yl; 3-ethoxycarbonylpyrazol-5-yl; 1-(morpholin-4-ylcarbonyl)-pyrazol-4-yl; 1,3-benzoxazol-6-yl; or 1-(pyridin-4-ylaminocarbonyl)-pyrazol-4-yl.
(3) With the above embodiments A, B, (I), (II), (III), (IV), (V), (a), (b), (c) and (d), (i) and (ii) and (1) and (2) and groups contained therein, in one group of compound, R³ is hydrogen, R⁴ is hydrogen, halo, alkoxy, carboxy, alkoxycarbonyl, aryl, heteroaryl or heterocyclyl where the aromatic or alicyclic ring in R⁴ is optionally substituted with R^{f} where R^{f} is alkyl, halo or alkoxy and R⁵ is hydrogen, alkyl, halo, hydroxyl or hydroxyalkyl; preferably R⁴ and R⁵ are hydrogen.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data. Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (I) where Z¹ is —N—, X is —NH— and other groups are as defined in the Summary of the Invention can be prepared as described in Scheme A below.

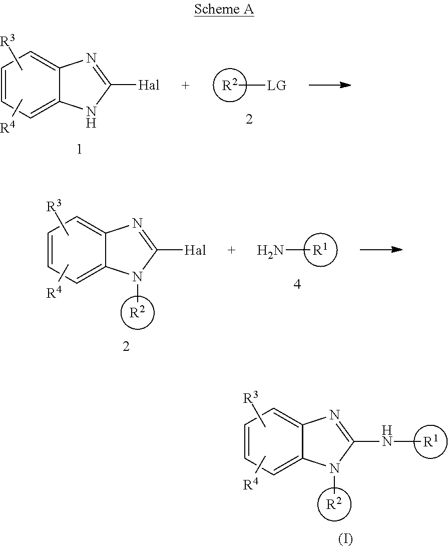

Treatment of a benzimidazole compound of formula 1 where Hal is a halo group such as chloro or bromo, with a compound of formula 2 where LG is a suitable leaving group such as halo under nucleophilic substitution reaction conditions provides a compound of formula 3. The reaction is carried out in the presence of a non-nucleophilic base such as DIPEA, pyridine, and the like in a suitable organic solvent such as tetrahydrofuran, and the like.

Compounds of formula 1 and 2 are either commercially available or they can be prepared by methods well known in the art. For example, compounds of formula 1 such as 2-chlorobenzimidazole, 2-chloro-5-methoxybenzimidazole, 2,5-dichloro-1H-benzoimidazole, 2-chloro-5,6-dimethylbenzimidazole, 2-chloro-5-nitro-1H-1,3-benzimidazole, 5-bromo-2-chloro-1H-1,3-benzimidazole, 2-chloro-5-(trifluoromethyl)benzimidazole, 2-chloro-4,5-dimethylbenzimidazole, 2-chloro-5-fluorobenzimidazole, 2-chloro-3H-benzoimidazole-5-carbonitrile, 2-chloro-1H-benzimidazole-5-sulfonyl chloride, 2-chloro-6-iodo-1H-benzoimidazole, and 2-chloro-4-methyl-1H-benzoimidazole are commercially available. Compounds of formula 2 such as 2,4-dichloro-6-alkyl-1,3,5-triazine can be prepared by reacting commercially available 2,4,6-trichloro-1,3,5-triazine with RMgX where R is an alkyl group. Compound of formula 2 such as 2,4-dichloro-6-methylpyrimidine, 2-amino-4-chloro-6-methylpyrimidine, 4-chloro-2-picoline, 2-anilino-4-chloro-6-methylpyrimidine, 4,6-dichloro-2-methylpyrimidine, 4-chloro-2-methylpyrimidine, 4-chloro-2,6-dimethylpyrimidine, N1-(4-chloro-6-methylpyrimidin-2-yl)-2,2-dimethylpropanamide, N-benzyl-4-chloro-6-methylpyrimidin-2-amine, 4-chloro-6-methylpyridin-2-amine, 4-chloro-2,6-dimethylpyridine, 4-chloro-6-methylpyrimidine, 4-chloro-2-isopropyl-6-methylpyrimidine, 4-chloro-2-methylpyridine hydrochloride, 4-chloro-2-methyl-6-trifluoromethylpyrimidine, 4-chloro-N-(4-chlorophenyl)-6-methyl-2-pyrimidinamine, 4-chloro-6-ethyl-2-methylpyrimidine, 4-amino-6-chloro-2-methylpyrimidine, 4-(N,N-dimethylamino)-6-chloro-2-methylpyrimidine, N-(4-chloro-6-methylpyrimidin-2-yl)-N-methyl, 4-chloro-n,6-dimethyl-2-pyrimidinamine, 4-chloro-N,N-6-trimethyl-2-pyrimidinamine, 2,4-dichloro-6-picoline, 4-chloro-6-methyl-2-propylpyrimidine, 4-chloro-6-methyl-2-trifluoromethylpyrimidine, 4,5-dichloro-2-methylpyridine, 4-chloro-5-iodo-2-methyl-pyrimidine, 4-chloro-5-ethyl-2-methylpyrimidine, 4-chloro-2,5-dimethylpyrimidine, (4-chloro-6-methylpyridin-2-yl)methanamine, 4-chloro-5-fluoro-2-methylpyrimidine, 3,4-dichloro-2-picoline, 5-bromo-4-chloro-2-picoline, 4-chloro-5-fluoro-2-methylpyridine, 4,5-dichloro-6-methylpyrimidine, 6-chloro-N-2-dimethyl-4-pyrimidinamine, and 2-[(6-chloro-2-methyl-4-pyrimidinyl)amino]-1-ethanol are commercially available. Compounds of formula 1 where R³ and R⁴ are halogen such as fluoro can be prepared as described in Working Example 2 below.

Treatment of compound of formula 3 with an amino compound of formula 4, then provides a compound of Formula (I). The reaction is carried out in a high boiling solvent such as DMSO, and the like and upon heating. Compounds of formula 4 such as 2-aminopyridine, 5-aminopyrazole, 3,4-dihydroxyaniline, 3-hydroxyaniline, 3-hydroxy-4-methoxyaniline, 6-aminoindazole, 5-aminoindole, 3-amino-4-fluoroindazole, 3-carboxyaniline, 3-aminoisoxazole, 4-fluoro-3-hydroxyaniline, 3-methoxyaniline, 3-hydroxy-4-methylaniline, 2,6-diaminopyridine, 1-methyl-3-aminopyrazole, and 5-amino-3-cyclopropylpyrazole are commercially available. Compounds of Formula (I) can be converted to other compounds of Formula (I) by methods well know in the art. Some such transformations are described below.

For example, compound of Formula (I) where R³ and/or R⁴ is aryl, heteroaryl, or cyano can be prepared from corresponding compound of Formula (I) where R³ and/or R⁴ is bromo, iodo or triflate. For example, treatment of a compound of Formula (I) where R³ and/or R⁴ is halo with an aryl or heteroarylboronic acid or aryl or heteroarylboronic esters employing a transition metal catalyst such as Pd(OAc)₂, Pd₂(dba)₃ or Pd(PPh₃)₄ with an appropriate ligand such as RuPhos or X-Phos provides a compound of Formula (I) substituted with an aryl or heteroaryl group. Treatment with CuCN provides a compound of Formula (I) where R³ and/or R⁴ is cyano. The cyano group can be hydrolyzed to give a carboxy group with can be converted to various carboxy derivatives such as alkoxycarbonyl, aminocarbonyl, hydroxymethyl, and alkoxymethyl, by methods well known in the art. For example, aminocarbonyl and alkoxycarbonyl can be prepared by coupling with alcohols and amines in the presence of a coupling agent such as EDCI or DCC and an amine base such as Hunig's base or TEA.

Alternatively, a compound of Formula (I) where R³ and/or R⁴ is halo can be converted to boronic acid or ester derivative and then can be reacted with aryl or heteroaryl halide under conditions described above to give a corresponding compound of Formula (I) where R³ and/or R⁴ is an aryl or heteroaryl ring. The boronic ester derivative of Formula (I) can also be reacted with an alcohol, phenol, or primary or secondary amides to prove a corresponding compound of Formula (I) where R³ and/or R⁴ is alkoxy, aryloxy, or acylamino group, respectively. The boronic ester can also be reacted with a peroxide such as hydrogen peroxide to give a corresponding compound of Formula (I) substituted with hydroxyl group which can then be converted to hydroxyalkoxy, aralkyloxy, heteroaralkoxy, or heterocyclylalkoxy groups using methods well known in the art.

Compounds of Formula (I) where R³ and/or R⁴ is amino, mono or disubstituted amino, and acylamino can be prepared by reacting a compound of Formula (I) where R³ or R⁴ is halo with a Pd(0) source and benzophenone imine to give the imine adduct which upon hydrolysis of the imine group provides the corresponding amine compound. Treatment of amine with a substituted thionylchloride or acid halide can give compounds of Formula (I) having the sulfonylamino or acylamino group, respectively. Aryl or heteroaryl substituted amines can be prepared by reacting the amine compound with aryl or heteroaryl halide in the presence of a transition metal catalyst such as Pd(OAc)₂, Pd₂(dba)₃ or Pd(PPh₃)₄ with an appropriate ligand such as RuPhos or X-Phos.

Compounds of Formula (I) where Z¹ is —N—, X is —NH— or —O— and other groups are as defined in the Summary of the Invention can be prepared as described in Scheme B below.

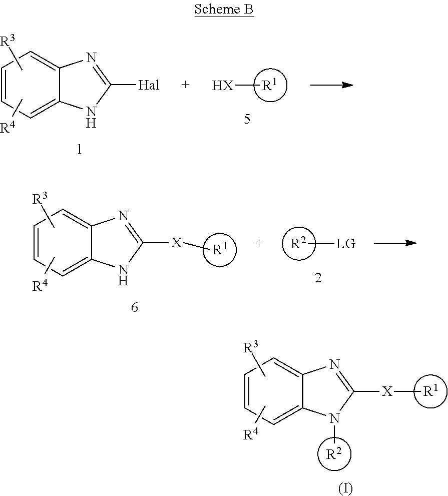

Treatment of a benzimidazole compound of formula 1 where Hal is a halo group such as chloro or bromo, with a compound of formula 5 where X is —NH— or —O— under nucleophilic substitution reaction conditions provides a compound of formula 6. The reaction is carried out in the presence of a non-nucleophilic base such as DIPEA, pyridine, and the like or inorganic base such as cesium carbonate and the like, in a suitable organic solvent such as alcoholic solvent, and the like.

Compounds of formula 1 and 5 are either commercially available or they can be prepared by methods well known in the art. Treatment of compound of formula 6 with a compound of formula 2 in the presence of a strong base such as sodium hydride in a suitable organic solvent such as tetrahydrofuran then provides a compound of Formula (I).

Compounds of Formula (I) where Z¹ is —CH—, X is —NH— or —O— and other groups are as defined in the Summary of the Invention can be prepared as described in Scheme C below.

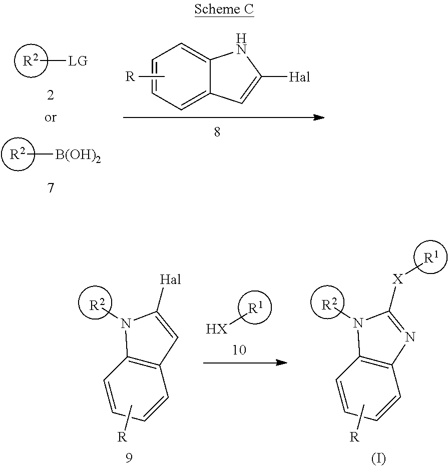

Base mediated nucleophilic addition of the indole halide of formula 8 with a compound of formula 2 provides a compound of formula 9. Alternatively, compound 9 can be prepared by addition of boronic acid of formula 7 where R² is as defined in the Summary of the Invention, to compound 8 in the presence of copper acetate and an amine base (see U.S. Pat. Appl. Publ., No. 2005054631). Compound 9 can be converted to compound of Formula (I) by reacting it with a compound of formula 10 where R1 and X are as defined in the Summary, under nucleophile aromatic substitution reaction conditions. The reaction is carried out in the presence of a non-nucleophilic base such as DIPEA, pyridine, and the like or inorganic base such as cesium carbonate and the like, in a suitable organic solvent such as alcoholic solvent, and the like. Alternatively, the reaction can employ amine 4 and a transition metal catalyst such as Pd(OAc)₂, Pd₂(dba)₃ or Pd(PPh₃)₄ with an appropriate ligand such as RuPhos or X-Phos.

### Utility

The compounds of the invention are kinase inhibitors, specifically PIK kinase inhibitors, more specifically, mTOR inhibitors and hence are useful in the treatment of cancers such as breast, lung, kidney, brain, ovarian, colon, cervical, endometrial, prostate, liver, thyroid, GI tract, blood and lymphoma and other diseases such as hamartoma syndromes, rheumatoid arthritis, and multiple sclerosis.

### Testing

The mTOR inhibitory activity of the compounds of the present invention can be tested using the in vitro described in Biological Example 1 below.

### Administration and Pharmaceutical Composition

In general, the compounds of this invention will be administered in a therapeutically effective amount by any of the accepted modes of administration for agents that serve similar utilities. Therapeutically effective amounts of compounds of Formula (I) may range from about 0.01 to about 500 mg per kg patient body weight per day, which can be administered in single or multiple doses. Preferably, the dosage level will be about 0.1 to about 250 mg/kg per day; more preferably about 0.5 to about 100 mg/kg per day. A suitable dosage level may be about 0.01 to about 250 mg/kg per day, about 0.05 to about 100 mg/kg per day, or about 0.1 to about 50 mg/kg per day. Within this range the dosage can be about 0.05 to about 0.5, about 0.5 to about 5 or about 5 to about 50 mg/kg per day.

For oral administration, the compositions are preferably provided in the form of tablets containing about 1.0 to about 1000 milligrams of the active ingredient, particularly about 1.0, 5.0, 10, 15, 20, 25, 50, 75, 100, 150, 200, 250, 300, 400, 500, 600, 750, 800, 900, and 1000 milligrams of the active ingredient. The actual amount of the compound of this invention, i.e., the active ingredient, will depend upon numerous factors such as the severity of the disease to be treated, the age and relative health of the subject, the potency of the compound utilized, the route and form of administration, and other factors.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

### EXAMPLES

The following preparations of compounds of Formula (I) and intermediates (References) are given to enable those skilled in the art to more clearly understand and to practice the present invention. They should not be considered as limiting the scope of the invention, but merely as being illustrative and representative thereof.

### Example 1

### Synthesis of 1-(4-amino-6-methyl-1,3,5-triazin-2-yl)-N-pyrimin-2-yl-1H-benzo[d]imidazol-2-amine

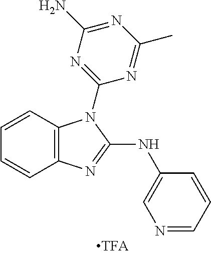

### Step 1

To a solution of 2,4,6-trichloro-1,3,5-triazine (20.4 g, 111 mmol) in DCM (221 mL) at 0° C., was added a solution of methylmagnesium bromide (3M) in ether (36.9 mL, 111 mmol) slowly over 15 minutes at 0° C. The resulting mixture turned bright yellow and was slowly warmed to room temperature. The solution was stirred overnight and then water was added and stirred for 5 minutes—slight exotherm. The resulting mixture was transferred to a sep. funnel containing water and the aqueous layer was washed with DCM. The organic layers were combined, dried with MgSO₄, filtered and concentrated to give 2,4-dichloro-6-methyl-1,3,5-triazine (16.1 grams, 89% yield). This material was taken onward to the following step without further purification.

### Step 2

To a solution of 2,4-dichloro-6-methyl-1,3,5-triazine (8.2 g, 50 mmol) and 2-chloro-1H-benzo[d]imidazole (7.6 g, 50 mmol) in THF (152 ml, 50 mmol) was added Hunig's base (9.6 ml, 55 mmol) at room temperature and the resulting mixture was stirred overnight. The reaction mixture was then concentrated and purified by the MPLC (100% DCM to 40% 90:10:1 DCM:MeOH:NH₄OH) to give 2-chloro-1-(4-chloro-6-methyl-1,3,5-triazin-2-yl)-1H-benzo[d]imidazole eluted with 100% DCM (10.2 g, 73% yield).

### Step 3

To 2-chloro-1-(4-chloro-6-methyl-1,3,5-triazin-2-yl)-1H-benzo[d]imidazole (4.7 g, 17 mmol) in a roundbottom flask was added ammonia in MeOH (7 N) (7.2 ml, 50 mmol) at room temperature. The resulting solution was stirred for 10 minutes and then concentrated to a white solid. The solid was then triturated with MeOH and filtered through a membrane filter to yield 4-(2-chloro-1H-benzo[d]imidazol-1-yl)-6-methyl-1,3,5-triazin-2-amine (3.2 grams). The liquid filtrate was concentrated and the trituration/filtration process was repeated to yield 700 mgs of clean compound (89% yield).

### Step 4

A solution of 4-(2-chloro-1H-benzo[d]imidazol-1-yl)-6-methyl-1,3,5-triazin-2-amine (0.058 g, 0.22 mmol) and 2-aminopyridine (0.021 g, 0.22 mmol), dissolved in DMSO (1.1 ml, 0.22 mmol) was heated to 135° C. for 25 minutes in the microwave. The resulting crude product was transferred directly to an HPLC sample tube and the crude mixture was purified by HPLC and the product was free-based with aqueous bicarbonate to provide 1-(4-amino-6-methyl-1,3,5-triazin-2-yl)-N-pyrimidin-2-yl-1H-benzo[d]imidazol-2-amine as a solid (5.9 mg, 8.3% yield). LCMS (formic acid modifier, ESI) m/z: 318.3 (M+1); 1H NMR (400 MHz, DMSO-d₆) δ ppm 3.12-3.21 (s, 3H); 7.05 (dd, J=7.34 Hz, 1H); 7.13 (dd, 1H); 7.23 (dd, 1 H); 7.35-7.47 (m, 3H); 7.91-8.04 (m, 3H); 8.15 (s, 1H); 8.58 (dd, J=8.07, 0.54 Hz, 1H); 11.67-11.89 (m, 1H).

### Example 2

### Synthesis of 1-(4-amino-6-methyl-1,3,5-triazin-2-yl)-5,6-difluoro-N-(1H-pyrazol-5-yl)-1H-benzo[d]imidazol-2-amine.TFA salt

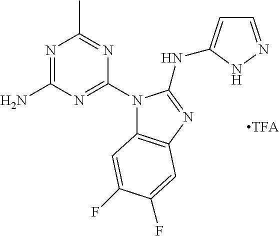

### Step 1

A sealable vial was charged with 1,2-diamino-4,5-difluorobenzene (66.4 mg, 461 μmol), THF (1 mL), then 1,1′-carbonyldimidazole (CDI) (112 mg, 691 μmol). The resulting reaction mixture was maintained at rt for 18 h. The solution was absorbed onto a 5 g silica loading cartridge and passed through a Redi-Sep® pre-packed silica gel column (12 g) using a gradient of 1% MeOH in CH₂Cl₂ to 10% MeOH in CH₂Cl₂ to afford 5,6-difluoro-1H-benzo[d]imidazol-2(3H)-one as a colorless solid, which was contaminated with CDI biproducts. The solid was transferred to a vial and phosphorus oxychloride (1.265 mL, 13.82 mmol) was added. The reaction mixture was stirred and heated at 90° C. for 18 h and then concentrated for purification by MPLC (Teledine Isco combiFlash Companion). The crude residue was taken up in minimal CH₂Cl₂ and absorbed onto a 5 g silica loading cartridge and passed through a Redi-Sep® pre-packed silica gel column (12 g) using a gradient of 2% EtOAc in hexanes to 90% EtOAc in hexanes to afford 2-chloro-5,6-difluoro-1H-benzo[d]imidazole (49.0 mg, 56.4% yield) as a colorless solid.

### Step 2

To a flask charged with 2-chloro-5,6-difluoro-1H-benzo[d]imidazole (49 mg, 260 μmol) was added 2,4-dichloro-6-methyl-1,3,5-triazine (43 mg, 260 μmol), 1,4-dioxane (15 mL, 0.4 M) followed by N,N-diisopropylethylamine (68 μl, 390 μmol). The reaction mixture was maintained at 35° C. for 18 h and then concentrated for purification by MPLC (Teledine Isco combiFlash Companion). The crude residue was taken up in minimal CH₂Cl₂ and absorbed onto a 25 g silica loading cartridge and passed through a Redi-Sep® pre-packed silica gel column (80 g) using a gradient of 1% MeOH in CH₂Cl₂ to 10% MeOH in CH₂Cl₂ to afford 2-chloro-1-(4-chloro-6-methyl-1,3,5-triazin-2-yl)-5,6-difluoro-1H-benzo[d]imidazole (29 mg, 35% yield) as a colorless solid.

### Step 3

To a flask charged with 2-chloro-1-(4-chloro-6-methyl-1,3,5-triazin-2-yl)-5,6-difluoro-1H-benzo[d]imidazole (29.0 mg, 92 μmol) was added CH₂Cl₂ (0.5 mL), followed by ammonia in MeOH, 7N (1311 μl, 9174 μmol). The reaction mixture was maintained at RT for 30 min, then concentrated. The residue was transferred to microwave reaction vessel and 5-aminopyrazole (7.6 mg, 92 μmol) was added, followed by 2-butanol (1.8 mL). The reaction mixture was heated at 130° C. in the microwave (Biotage Initiator) for 10 min. The crude reaction mixture was diluted with minimal MeOH/DMSO and purified by preparative HPLC (Gilson: 5-90% (0.1% TFA in CH₃CN) in H₂O over 15 min). Clean fractions were combined and concentrated to afford 1-(4-amino-6-methyl-1,3,5-triazin-2-yl)-5,6-difluoro-N-(1H-pyrazol-5-yl)-1H-benzo[d]imidazol-2-amine 2,2,2-trifluoroacetate (21 mg, 50% yield) as colorless solid. LCMS (formic acid modifier, ESI) m/z: 344.2 (M+1); 1H NMR (400 MHz, DMSO-d₆) δ ppm 2.46 (s, 3H); 6.77 (d, J=2.15 Hz, 1H); 7.43 (dd, J=10.95, 7.63 Hz, 1H); 7.59-7.65 (m, J=18.19 Hz, 1H); 7.70 (d, J=2.25 Hz, 1H); 8.03 (s, 1H); 8.21 (s, 1H); 8.62 (dd, J=11.88, 7.97 Hz, 1H); 11.69 (s, 1H).

### Example 3

### Synthesis of 1-(4-amino-6-methyl-1,3,5-triazin-2-yl)-N-(1H-pyrazol-5-yl)-1H-benzo[d]imidazol-2-amine

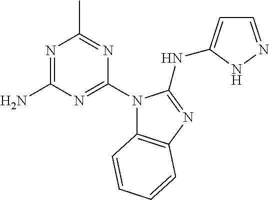

To a microwave vessel charged with 4-(2-chloro-1H-benzo[d]imidazol-1-yl)-6-methyl-1,3,5-triazin-2-amine (770 mg, 2954 μmol) and 5-aminopyrazole (295 mg, 3545 μmol) was added 2-butanol (10 mL). The reaction vessel was sealed and heated in the microwave (Biotage Initiator) at 135° C. for 10 min. After cooling, a white solid precipitated from the reaction. The solid was collected by vacuum filtration and then slurried in 1:1 isopropanol (IPA) (5 mL):saturated aqueous NaHCO₃ (5 mL) and stirred for 18 h at RT under positive nitrogen flow. The slurry was then heated at 60° C. for 2 h (until gas evolution ceased). The reaction mixture was cooled and the suspended solid was collected by vacuum filtration, washing with water (5 mL) and IPA (10 mL). The solid was further dried under reduced pressure to give 1-(4-amino-6-methyl-1,3,5-triazin-2-yl)-N-(1H-pyrazol-5-yl)-1H-benzo[d]imidazol-2-amine (626.0 mg, 69.0% yield) as a white powder. LCMS (formic acid modifier, ESI) m/z: 308.4 (M+1); 1H NMR (400 MHz, DMSO-d₆) δ ppm 2.46 (s, 3H); 6.82 (d, J=1.76 Hz, 1H); 7.06-7.13 (m, 1H); 7.18-7.24 (m, 1H); 7.38 (d, J=7.14 Hz, 1H); 7.68 (d, J=2.05 Hz, 1H); 7.95 (s, 1H); 8.07 (s, 1H); 8.50-8.66 (m, 1H); 11.61 (s, 1H); 12.37 (s, 1H).

### Example 4

### Synthesis of 3-(1-(2-chloro-6-methylpyrimidin-4-yl)-1H-benzo[d]imidazol-2-yloxy)phenol

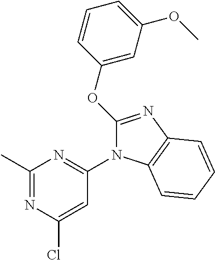

### Step 1

Cesium carbonate (2.11 g, 6.60 mmol) was added to a solution of 2-chloro-benzoimidazole (0.500 g, 3.32 mmol) and 3-methoxyphenol (3.32 g, 2.81 mL, 26.3 mmol) in isopropanol (10 mL). The reaction mixture was heated at 150° C. for 17 h and was then cooled to room temperature. The reaction mixture was partitioned between ethyl acetate and 2.0 N sodium hydroxide solution. The aqueous phase was separated and extracted with ethyl acetate. The combined organic phases were washed with brine, dried over anhydrous magnesium sulfate, filtered, and concentrated to afford an off-white solid. Trituration with ethyl acetate and filtering afforded 2-(3-methoxyphenoxy)-1H-benzo[d]imidazole as a white solid.

### Step 2

Sodium hydride (60% dispersion in mineral oil, 0.025 g, 1.04 mmol) was added to a solution of 2-(3-methoxyphenoxy)-1H-benzo[d]imidazole (0.0.250 g, 1.04 mmol) in N,N-dimethylformamide (10 mL). 4,6-Dichloro-2-methylpyrimidine (0.161 g, 0.989 mmol) was added and the reaction mixture was stirred at room temperature for 16 h. Saturated ammonium chloride solution was added and the reaction mixture was partitioned between dichloromethane and water. The aqueous phase was separated and extracted with dichloromethane. The combined organic phases were washed with brine, dried over anhydrous magnesium sulfate, filtered, and concentrated to afford a brown solid. This solid was purified via column chromatography on silica gel (eluting with 0-100% 90% DCM/10% MeOH/1% NH₄OH-DCM) to afford 1-(6-chloro-2-methylpyrimidin-4-yl)-2-(3-methoxyphenoxy)-1H-benzo[d]imidazole as a white solid.

### Example 5

### Synthesis of 4-(2-(3-methoxyphenoxy)-1H-benzo[d]imidazol-1-yl)-6-methylpyrimidin-2-amine

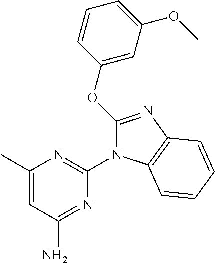

A resealable tube was charged with 1-(2-chloro-6-methylpyrimidin-4-yl)-2-(3-methoxyphenoxy)-1H-benzo[d]imidazole (0.100 g, 0.270 mmol) and ammonium hydroxide (0.48 g, 0.53 mL, 14.0 mmol). The tube was sealed and stirred at room temperature for 3 hours. The reaction mixture was concentrated to an oil and then diluted with MeOH (3 ml). The reaction mixture was purified by preparative HPLC (Gilson: 10-90% (0.1% TFA in MeCN) in water over 20 min). Clean fraction were combined and passed through a 2 g Isolute SCX-2 column. MeOH (10 mL) was passed through the column and discarded. The column was washed with NH₃ (2N solution MeOH, 20 mL). The filtrate was concentrated, triturated with diethyl ether, and filtered to afford 4-(2-(3-methoxyphenoxy)-1H-benzo[d]imidazol-1-yl)-6-methylpyrimidin-2-amine as an off white solid. MS (MH⁺) 348.2; Calculated 347.37 for C₁₉H₁₇N₅O₂; 1H NMR (400 MHz, DMSO-d₆) δ ppm 2.34 (s, 3H); 3.79 (s, 3H); 6.84-6.87 (m, 1H); 6.90-6.95 (m, 2H); 6.98 (d, J=0.29 Hz, 1H); 7.02-7.05 (m, 1H); 7.10 (d, J=2.25 Hz, 1H); 7.21-7.24 (m, 2H); 7.44-7.47 (m, 1H); 8.17-8.20 (m, 1H).

### Example 6

### Synthesis of 3-(1-(2-amino-6-methylpyrimidin-4-yl)-1H-benzo[d]imidazol-2-yloxy)phenol

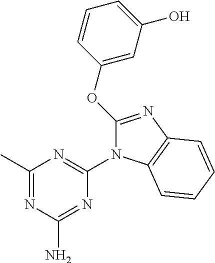

A solution of 4-(2-(3-methoxyphenoxy)-1H-benzo[d]imidazol-1-yl)-6-methyl-pyrimidin-2-amine (0.025 g, 0.072 mmol) in dichloromethane (2 mL) was cooled to 0° C. and boron tribromide (1.0 M in DCM, 0.018 g, 0.072 mL, 0.072 mmol) was added. The reaction mixture was stirred at 0° C. for 3 hours and allowed to warm to room temperature. The reaction mixture was concentrated to an oil and then diluted with MeOH (3 ml). The reaction mixture was purified by preparative HPLC (Gilson: 10-90% (0.1% TFA in MeCN) in water over 20 min). Clean fraction were combined and passed through a 2 g Isolute SCX-2 column. MeOH (10 mL) was passed through the column and discarded. The column was washed with NH₃ (2N solution MeOH, 20 mL). The filtrate was concentrated, triturated with diethyl ether, and filtered to afford 3-(1-(2-amino-6-methylpyrimidin-4-yl)-1H-benzo[d]imidazol-2-yloxy)phenol as an off white solid. MS (MH⁺) 334.2; Calculated 333.34 for C18H15N5O2; 1H NMR (400 MHz, DMSO-d₆) δ ppm 2.33 (s, 3H); 6.70-6.74 (m, 1H;) 6.83-6.88 (m, 2 H); 6.94 (d, J=0.29 Hz, 1H;) 6.96 (s, 2H); 7.20-7.25 (m, 2H); 7.44-7.47 (m, 1H); 8.16-8.20 (m, 1H); 9.86 (s, 1H).

### Example 7

### Synthesis of methyl 1-(4-amino-6-methyl-1,3,5-triazin-2-yl)-2-(3-methoxyphenylamino)-1H-benzo[d]imidazole-5-carboxylate

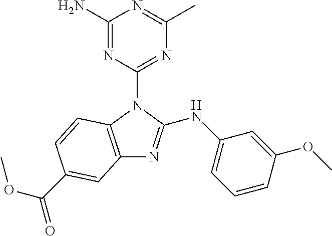

### Step 1

In a 50 mL round bottom flask, dissolved methyl 3,4-diaminobenzoate (1.00 g, 6.02 mmol) in THF (6.00 mL) and after adding 3-methoxyphenyl isothiocyanate (0.994 g, 6.02 mmol) the reaction mixture was stirred at 20° C. for 2 hours. HATU (2.75 g, 7.22 mmol) and N,N-diisopropylethylamine (2.09 mL, 12.0 mmol) were added and the stirring was continued. After 3 hours the crude product methyl 2-(3-methoxyphenylamino)-1H-benzo[d]imidazole-5-carboxylate was used in the next step without further purification.

### Step 2

In a 20 mL sealed tube, dissolved methyl 2-(3-methoxyphenylamino)-1H-benzo[d]imidazole-5-carboxylate (0.750 g, 2.52 mmol) in THF (4.00 mL). 2,4-Dichloro-6-methyl-1,3,5-triazine (0.414 g, 2.52 mmol) and N-ethyl-N-isopropylpropan-2-amine (0.879 mL, 5.05 mmol) were added and the reaction mixture was stirred at 20° C. for 24 hours. Ammonia, 2.0 M in methanol (1.26 mL, 2.52 mmol) was added and the reaction mixture was stirred at room temperature for 1 hour. The reaction mixture was concentrated down and purified using Gilson reverse phase chromatography. The eluent was extracted into dichloromethane, washed with sodium carbonate, H₂O, dried with Na₂SO₄, filtered through fritted funnel, concentrated to yield methyl 3-(4-amino-6-methyl-1,3,5-triazin-2-yl)-2-(3-methoxyphenylamino)-3H-benzo[d]imidazole-5-carboxylate as a light yellow solid. MS [M+H]=406.0; Calc'd 405.4 for C₂₀H₁₉N₂O₃.

### Example 8

### Synthesis of (1-(4-amino-6-methyl-1,3,5-triazin-2-yl)-2-(3-methoxyphenylamino)-1H-benzo[d]imidazol-5-yl)methanol

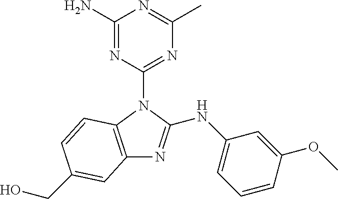

### Step 1

In a 20 mL sealed tube, dissolved methyl 2-(3-methoxyphenylamino)-1H-benzo[d]imidazole-5-carboxylate (0.300 g, 1.009 mmol) in THF (2.00 mL). The reaction mixture was cooled to 0° C. Lithium aluminum hydride (0.191 g, 5.045 mmol) was added and stirring was continued at 0° C. for 2 hours and then the reaction mixture was allowed to warm to room temperature. The reactin mixture was slowly added to a mixture of ice and saturated ammonium chloride. Gray solid crashed out which was filtered through a pad of celite. The product was extracted into ethyl acetate. The extracts were washed with water, dried with Na₂SO₄, filtered through fritted funnel, and concentrated down. Purification by silica gel chromatography using 0-100% CH₂Cl₂:MeOH(90:10)/CH₂Cl₂ gave (2-(3-methoxyphenylamino)-1H-benzo[d]imidazol-5-yl)methanol (0.130 g) as tan solid.

### Step 2

In a 20 mL sealed tube, (2-(3-methoxyphenylamino)-1H-benzo[d]imidazol-5-yl)methanol (0.130 g, 0.483 mmol) in THF (4.00 mL) was dissolved and 2,4-dichloro-6-methyl-1,3,5-triazine (87 mg, 531 mmol) was added. The reaction mixture was stirred at 20° C. for 24 hours. Ammonia, 2.0 M in methanol (0.241 mL, 0.483 mmol) was added and the reaction mixture was stirred at room temperature for 1 hour. The crude was purified by Gilson reverse phase chromatography. The eluent was extracted into dichloromethane, washed with sodium carbonate, H₂O, dried over Na₂SO₄, filtered through fritted funnel, concentrated down to yield a mixture of regioisomers which were separated by prep. HPLC to give (1-(4-amino-6-methyl-1,3,5-triazin-2-yl)-2-(3-methoxyphenylamino)-1H-benzo[d]imidazol-5-yl)methanol as a light yellow solid. MS [M+H]=378.0; Calc'd 377.4 for C₁₉H₁₉N₇O₂.

### Example 9

### Synthesis of 1-(4-methyl-6-(methylamino)-1,3,5-triazin-2-yl)-6-(2-methylpyridin-4-yl)-N-(1H-pyrazol-3-yl)-1H-benzo[d]imidazol-2-amine 2,2,2-trifluoroacetate and 1-(4-methyl-6-(methylamino)-1,3,5-triazin-2-yl)-5-(2-methylpyridin-4-yl)-N-(1H-pyrazol-3-yl)-1H-benzo[d]imidazol-2-amine 2,2,2-trifluoroacetate

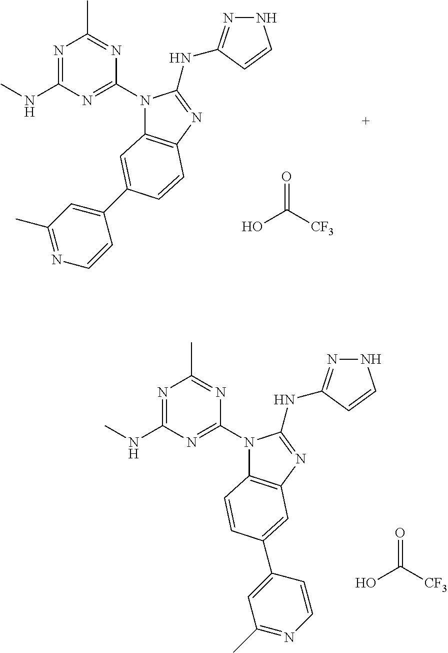

### Step 1

Followed procedure in Example 1, step 2 using dioxane as the solvent and 5-bromo-2-chloro-1H-benzo[d]imidazole to make 6-bromo-2-chloro-1-(4-chloro-6-methyl-1,3,5-triazin-2-yl)-1H-benzo[d]imidazole and 5-bromo-2-chloro-1-(4-chloro-6-methyl-1,3,5-triazin-2-yl)-1H-benzo[d]imidazole (2.86 g, 41% yield) as a 1:1 mixture of regioisomers.

### Step 2

To a solution of 6-bromo-2-chloro-1-(4-chloro-6-methyl-1,3,5-triazin-2-yl)-1H-benzo[d]imidazole and 5-bromo-2-chloro-1-(4-chloro-6-methyl-1,3,5-triazin-2-yl)-1H-benzo[d]imidazole as a 1:1 mixture of regioisomers (2.86 g, 8 mmol) in 1,4-dioxane (40 mL) was added methylamine, 2.0 M solution in THF (8 mL, 1.59 mmol) at room temperature and the resulting mixture was stirred overnight. The reaction mixture was concentrated to yield 4-(6-bromo-2-chloro-1H-benzo[d]imidazol-1-yl)-N,6-dimethyl-1,3,5-triazin-2-amine and 4-(5-bromo-2-chloro-1H-benzo[d]imidazol-1-yl)-N,6-dimethyl-1,3,5-triazin-2-amine (2.542 g, 90% yield).

### Step 3

To a microwave vessel charged with 4-(6-bromo-2-chloro-1H-benzo[d]imidazol-1-yl)-N,6-dimethyl-1,3,5-triazin-2-amine and 4-(5-bromo-2-chloro-1H-benzo[d]imidazol-1-yl)-N,6-dimethyl-1,3,5-triazin-2-amine as a 1:1 mixture of regioisomers (2.542 g, 7.2 mmol) and 3-aminopyrazole (0.597 mL, 7.2 mmol) was added 2-butanol (36 mL). The reaction vessel was sealed and heated in the microwave (Biotage Initiator) at 100° C. for 15 min. The solid was collected by vacuum filtration to yield pure 6-bromo-1-(4-methyl-6-(methylamino)-1,3,5-triazin-2-yl)-N-(1H-pyrazol-3-yl)-1H-benzo[d]imidazol-2-amine hydrochloride and 5-bromo-1-(4-methyl-6-(methylamino)-1,3,5-triazin-2-yl)-N-(1H-pyrazol-3-yl)-1H-benzo[d]imidazol-2-amine hydrochloride (3.00 g, 96% yield) as a yellow solid.

### Step 4

To a sealable vessel charged with 6-bromo-1-(4-methyl-6-(methylamino)-1,3,5-triazin-2-yl)-N-(1H-pyrazol-3-yl)-1H-benzo[d]imidazol-2-amine hydrochloride and 5-bromo-1-(4-methyl-6-(methylamino)-1,3,5-triazin-2-yl)-N-(1H-pyrazol-3-yl)-1H-benzo[d]imidazol-2-amine hydrochloride as a 1:1 mixture of regioisomers (200 mg, 4.58 mmol), 1,1-bis(diphenylphosphino)ferrocene]dichloropalladium (37.4 mg, 46 μmol), and 2-methylpyridin-4-ylboronic acid (157 mg, 1.15 mmol) was added 1,4-dioxane (2.3 mL) followed by sodium carbonate (194 mg, 1.83 mmol) as a 2 M solution in water. The reaction vessel was sealed and heated on a hot plate at 80° C. for 16 h. Crude material was filtered through Celite washing with methanol (20 mL). The filtrate was concentrated and was diluted with minimal MeOH/DMSO and purified by preparative HPLC (Gilson: 5-90% (0.1% TFA in CH₃CN) in H₂O over 15 min). Clean fractions were combined and concentrated to afford 1-(4-methyl-6-(methylamino)-1,3,5-triazin-2-yl)-6-(2-methylpyridin-4-yl)-N-(1H-pyrazol-3-yl)-1H-benzo[d]imidazol-2-amine 2,2,2-trifluoroacetate and 1-(4-methyl-6-(methylamino)-1,3,5-triazin-2-yl)-5-(2-methylpyridin-4-yl)-N-(1H-pyrazol-3-yl)-1H-benzo[d]imidazol-2-amine 2,2,2-trifluoroacetate (4.5 mg, 5% yield) as a brown solid. LCMS (formic acid modifier, ESI) m/z: 413.4 (M+1). ¹H NMR (400 MHz, DMSO-d₆) δ ppm 2.64-2.82 (m, 3H); 2.86-3.12 (m, 3H); 3.57 (s, 1H); 6.76-6.95 (m, 1H); 7.57 (d, J=7.92 Hz, 1H); 7.67-8.49 (m, 5H); 8.52-8.92 (m, 3H); 9.09 (br s, 1H); 11.58-12.21 (m, 1H).

### Example 10

### Synthesis of 4-(2-(1H-pyrazol-3-ylamino)-1-(4-methyl-6-(methylamino)-1,3,5-triazin-2-yl)-1H-benzo[d]imidazol-6-yl)-N-methylbenzamide 2,2,2-trifluoroacetate and 4-(2-(1H-pyrazol-3-ylamino)-1-(4-methyl-6-(methylamino)-1,3,5-triazin-2-yl)-1H-benzo[d]imidazol-5-yl)-N-methylbenzamide 2,2,2-trifluoroacetate

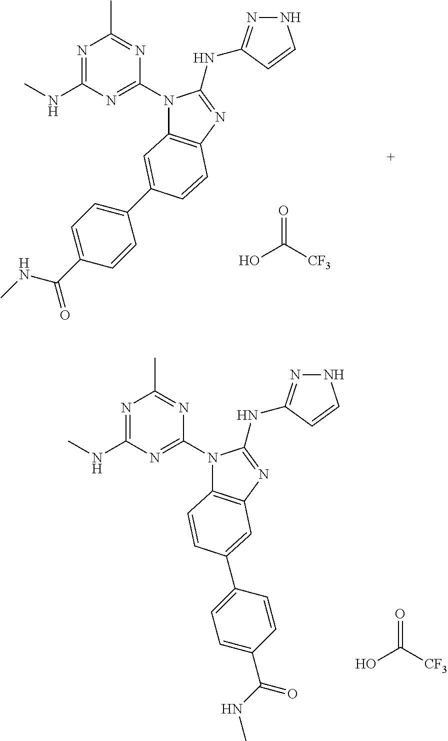

### Step 1

Following the procedure Example 9, Step 4 using 4-(methylcarbamoyl)phenylboronic acid instead of 2-methylpyridin-4-ylboronic acid provided 4-(2-(1H-pyrazol-3-ylamino)-1-(4-methyl-6-(methylamino)-1,3,5-triazin-2-yl)-1H-benzo[d]imidazol-6-yl)-N-methylbenzamide 2,2,2-trifluoroacetate and 4-(2-(1H-pyrazol-3-ylamino)-1-(4-methyl-6-(methylamino)-1,3,5-triazin-2-yl)-1H-benzo[d]imidazol-5-yl)-N-methylbenzamide 2,2,2-trifluoroacetate (15 mg, 5.7% yield). LCMS (trifluoroacetic acid modifier, ESI) m/z: 455.0 (M+1). ¹H NMR (400 MHz, DMSO-d₆) δ ppm 2.81 (br s, 3H); 2.91-3.16 (m, 4H); 6.74-6.90 (m, 1H); 7.52 (br. s., 1H); 7.62 (d, J=7.53 Hz, 1H); 7.69-7.88 (m, 4H); 7.89-8.02 (m, 2H); 8.31-8.74 (m, 3H); 8.79-9.06 (m, 1H); 11.76-12.14 (m, 1H).

### Example 11

### Synthesis of 1-(4-(cyclopropylmethylamino)-6-methyl-1,3,5-triazin-2-yl)-N-(1H-pyrazol-3-yl)-1H-benzo[d]imidazol-2-amine 2,2,2-trifluoroacetate

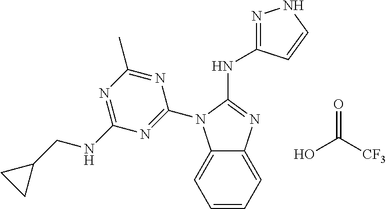

### Step 1

To a round-bottomed flask was added 2-chloro-1-(4-chloro-6-methyl-1,3,5-triazin-2-yl)-1H-benzo[d]imidazole (0.200 g, 7.14 mmol) and cyclopropane methylamine (0.073 mL, 8.57 mmol) in methanol (3.5 mL). The reaction mixture stirred for 15 min and solid was isolated by vacuum filtration to afford 4-(2-chloro-1H-benzo[d]imidazol-1-yl)-N-(cyclopropylmethyl)-6-methyl-1,3,5-triazin-2-amine (0.154 g, 68.5% yield) as a white solid.

### Step 2

To a microwave reaction vessel charged with 4-(2-chloro-1H-benzo[d]imidazol-1-yl)-N-(cyclopropylmethyl)-6-methyl-1,3,5-triazin-2-amine (0.154 g, 0.489 mmol) and 3-aminopyrazole (0.045 g, 0.538 mmol) was added 2-butanol (4.89 mL). The reaction vessel was sealed and heated in the microwave (Biotage Initiator) at 135° C. for 10 min. Purification was done by preparative HPLC (Gilson: 10-90% (0.1% TFA in CH₃CN) in H₂O over 15 min). Clean fractions were combined and concentrated to afford 1-(4-(cyclopropylmethylamino)-6-methyl-1,3,5-triazin-2-yl)-N-(1H-pyrazol-3-yl)-1H-benzo[d]imidazol-2-amine 2,2,2-trifluoroacetate (0.039 g, 16.8% yield) as a white solid. LCMS (formic acid modifier, ESI) m/z: 362.4 (M+1). ¹H NMR (400 MHz, DMSO-d₆) δ ppm 0.31 (d, J=5.58 Hz, 2H); 0.51 (d, J=8.22 Hz, 2H); 1.02-1.26 (m, 1H); 3.21-3.44 (m, 2H); 6.57-6.77 (m, 1H); 7.13-7.40 (m, 2H); 7.52 (dd, J=19.71, 7.38 Hz, 1H); 7.67-7.98 (m, 1H); 8.48 (d, J=7.63 Hz, 1H); 8.60-9.03 (m, 2H); 12.15 (br s, 1H).

### Example 12

### Synthesis of 2-(4-(2-(1H-pyrazol-3-ylamino)-1H-benzo[d]imidazol-1-yl)-6-methyl-1,3,5-triazin-2-ylamino)ethanol hydrochloride

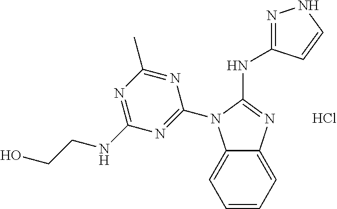

### Step 1

Following procedure in Example 11, Step 1 using ethanolamine instead of cyclopropanemethylamine provided 2-(4-(2-chloro-1H-benzo[d]imidazol-1-yl)-6-methyl-1,3,5-triazin-2-ylamino)ethanol hydrochloride (250 mg, 92% yield).

### Step 2

Following procedure in Example 11, Step 2 provided 2-(4-(2-(1H-pyrazol-3-ylamino)-1H-benzo[d]imidazol-1-yl)-6-methyl-1,3,5-triazin-2-ylamino)ethanol hydrochloride (20 mg, 7% yield). LCMS (trifluoroacetic acid modifier, ESI) m/z: 352.0 (M+1). ¹H NMR (400 MHz, DMSO-d₆) δ ppm 2.42-2.48 (m, 3H) 3.45-3.75 (m, 4H) 4.80 (br. s., 0.5H) 4.95 (br. s., 0.5H) 6.78-6.92 (m, 1H) 7.13 (d, J=7.43 Hz, 1H) 7.22 (t, J=6.11 Hz, 1H) 7.31-7.51 (m, 1H) 7.70 (br. s., 1H) 8.36-8.74 (m, 2H) 11.56 (br. s., 0.5H) 11.80 (br. s., 0.5H) 12.27-12.53 (m, 1H)

### Example 13

### Synthesis of 1-(4-methyl-6-(2-(piperazin-1-yl)ethylamino)-1,3,5-triazin-2-yl)-N-(1H-pyrazol-3-yl)-1H-benzo[d]imidazol-2-amine 2,2,2-trifluoroacetate

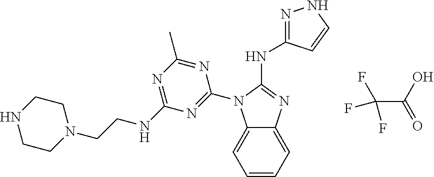

### Step 1

Following procedure in Example 11, Step 1 using 4-(2-amino-ethyl)-piperazine-1-carboxylic acid tert-butyl ester provided tert-butyl 4-(2-(4-(2-chloro-1H-benzo[d]imidazol-1-yl)-6-methyl-1,3,5-triazin-2-ylamino)ethyl)piperazine-1-carboxylate (250 mg, 99% yield).

### Step 2

Following procedure in Example 11, Step 2 provided tert-butyl 4-(2-(4-(2-(1H-pyrazol-3-ylamino)-1H-benzo[d]imidazol-1-yl)-6-methyl-1,3,5-triazin-2-ylamino)ethyl)piperazine-1-carboxylate 2,2,2-trifluoroacetate (20 mg, 6% yield).

### Step 3

Treatment of 1H-benzo[d]imidazol-1-yl)-6-methyl-1,3,5-triazin-2-ylamino)ethyl)piperazine-1-carboxylate 2,2,2-trifluoroacetate with TFA afforded 1-(4-methyl-6-(2-(piperazin-1-yl)ethylamino)-1,3,5-triazin-2-yl)-N-(1H-pyrazol-3-yl)-1H-benzo[d]imidazol-2-amine 2,2,2-trifluoroacetate (13 mg, 17% yield).

LCMS (trifluoroacetic acid modifier, ESI) m/z: 420.0 (M+1). ¹H NMR (400 MHz, DMSO-d₆) δ ppm 2.80-3.42 (m, 10H); 3.69 (br s, 2H); 6.67-6.91 (m, 1H); 7.05-7.34 (m, 2H); 7.44 (d, J=6.36 Hz, 1H); 7.73 (br s, 1H); 8.20-9.16 (m, 3H); 11.50-12.00 (m, 1H).

### Example 14

### Synthesis of 3-(1-(2-amino-6-methylpyrimidin-4-yl)-1H-benzo[d]imidazol-2-yloxy)phenol

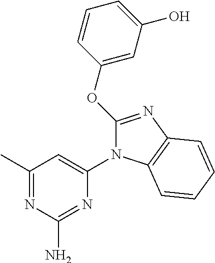

### Step 1

Cesium carbonate (2.11 g, 6.60 mmol) was added to a solution of 2-chloro-benzoimidazole (0.500 g, 3.32 mmol) and 2-methoxyphenol (3.32 g, 2.81 mL, 26.3 mmol) in isopropanol (10 mL). The reaction mixture was heated at 150° C. for 17 h and was then cooled to room temperature. The reaction mixture was partitioned between ethyl acetate and 2.0 N sodium hydroxide solution. The aqueous phase was separated and extracted with ethyl acetate. The combined organic phases were washed with brine, dried over anhydrous magnesium sulfate, filtered, and concentrated to afford an off-white solid. Trituration with ethyl acetate and filtering afforded 2-(3-methoxyphenoxy)-1H-benzo[d]imidazole as a white solid.

### Step 2

Sodium hydride (60% dispersion in mineral oil, 0.025 g, 1.04 mmol) was added to a solution of 2-(3-methoxyphenoxy)-1H-benzo[d]imidazole (0.0.250 g, 1.04 mmol) in N,N-dimethylformamide (10 mL). 4,6-Dichloro-2-methylpyrimidine (0.161 g, 0.989 mmol) was added and the mixture stirred at room temperature for 16 h. Saturated ammonium chloride solution was added and the mixture was partitioned between dichloromethane and water. The aqueous phase was separated and extracted with dichloromethane. The combined organic phases were washed with brine, dried over anhydrous magnesium sulfate, filtered, and concentrated to afford a brown solid. This solid was purified via column chromatography on silica gel (eluting with 0-100% 90% DCM/10% MeOH/1% NH₄OH-DCM) to afford 1-(6-chloro-2-methylpyrimidin-4-yl)-2-(3-methoxyphenoxy)-1H-benzo[d]imidazole as a white solid.

### Step 3

A resealable tube was charged with 1-(2-chloro-6-methylpyrimidin-4-yl)-2-(3-methoxyphenoxy)-1H-benzo[d]imidazole (0.100 g, 0.270 mmol) and ammonium hydroxide (0.48 g, 0.53 mL, 14.0 mmol). The tube was sealed and stirred at room temperature for 3 hours. The reaction mixture was concentrated to an oil and then diluted with MeOH (3 mL). The reaction mixture was purified by preparative HPLC (Gilson: 10-90% (0.1% TFA in MeCN) in water over 20 min). Clean fraction were combined and passed through a 2 g Isolute SCX-2 column. MeOH (10 mL) was passed through the column and discarded. The column was washed with NH₃ (2N solution MeOH, 20 mL). The filtrate was concentrated, triturated with diethyl ether, and filtered to afford 4-(2-(3-methoxyphenoxy)-1H-benzo[d]imidazol-1-yl)-6-methylpyrimidin-2-amine as an off white solid

### Step 4

A solution of 4-(2-(3-methoxyphenoxy)-1H-benzo[d]imidazol-1-yl)-6-methylpyrimidin-2-amine (0.025 g, 0.072 mmol) in dichloromethane (2 mL) was cooled to 0° C. and boron tribromide (1.0 M in DCM, 0.018 g, 0.072 mL, 0.072 mmol) was added. The reaction mixture was stirred at 0° C. for 3 h and allowed to warm to room temperature. The reaction mixture was concentrated to an oil and then diluted with MeOH (3 mL) and purified by preparative HPLC (Gilson: 10-90% (0.1% TFA in MeCN) in water over 20 min). Clean fraction were combined and passed through a 2 g Isolute SCX-2 column. MeOH (10 mL) was passed through the column and discarded. The column was washed with NH₃ (2N solution MeOH, 20 mL). The filtrate was concentrated, triturated with diethyl ether, and filtered to afford 3-(1-(2-amino-6-methylpyrimidin-4-yl)-1H-benzo[d]imidazol-2-yloxy)phenol as an off white solid. MS (MH⁺) 334.2; Calculated 333.34 for C₁₈H₁₅N₅O₂; 1H NMR (400 MHz, DMSO-d₆) δ ppm 2.33 (s, 3H); 6.70-6.74 (m, 1H;) 6.83-6.88 (m, 2H); 6.94 (d, J=0.29 Hz, 1H;) 6.96 (s, 2H); 7.20-7.25 (m, 2H); 7.44-7.47 (m, 1H); 8.16-8.20 (m, 1H); 9.86 (s, 1 H).

### BIOLOGICAL EXAMPLES

### Example 1

### In vitro assays

### mTOR LanthaScreen

The mTOR LanthaScreen is a TR-FRET assay measuring the phosphorylation of mTOR's substrate 4EBP1. 384 well compound plates were prepared containing 1 μl of compound per well starting at 5 mM and diluted 1:2 across the row, resulting in a 22 well serial dilution. 24 μl of assay buffer (Invitrogen, PV4794) with 2 mM DTT was added to the compound plate in rows 1-24 using the VELOCITY1 1™ VPREP™ 384 ST resulting in a DMSO concentration of 4%. The compound plate was mixed and 2.5 μl of serially diluted compound or controls was added to the assay plate (Costar, 3658).

The assay was conducted on the PerkinElmer® FlexDrop PLUS. A 5 μl mix of 800 nM GFP-4E-BP1 (Invitrogen, PV4759) and 20 μM ATP (Amgen) was added to rows 1-24. 2.5 μl of 0.6 μg/ml of mTOR Enzyme (Amgen) was added to rows 1-23. 2.5 μl of assay buffer was added to row 24 for the low control. The final concentration of the compounds was 50 μM serially diluted to 23.84 pM in 1% DMSO. The final high control had 1% DMSO and the low control was a no enzyme control with a concentration of 1% DMSO. The final concentrations in the assay reagents were 400 nM GFP-4E-BP1, 10 μM of ATP and 0.15 μg/ml of mTOR enzyme. The compound, enzyme, and substrate incubate for 90 minutes. At this point, 10 μl of stop solution was added (20 mM Tris, pH 7.5 (Invitrogen, 15567-027), 0.02% Sodium Azide (Teknova, S0208), 0.01% NP-40 (Roche, 11754599001), 20 mM EDTA (Invitrogen, 15575-038) and 4 nM of Tb-anti-p4E-BP1 (Invitrogen, PV4758)) for a final concentration of 2 nM of Tb-anti-p4E-BP1.

Sixty minutes later the plates were read on the PerkinElmer® EnVision™ 2103. Multilable Reader using the Excitation filter 340 nm and the Emission filters 520 nm and 495 nm. The ratio of 520 nm/495 nm was calculated and the POC data was analyzed to report the IC₅₀ IP for the phosphorylation of 4EBP1.

### p4EBP 1 AlphaScreen

The p4EBP 1 AlphaScreen assay determines whether there is phosphorylation of 4EBP 1 at Thr37/Thr46 by recruitment of a phosphospecific antibody. This assay was performed using U87 MG cells. The U87 growth media consists of MEM (Gibco, 51200-038) supplemented with 10% FBS (Gibco, 16140-071), 1× Non-Essential Amino Acids (Gibco, 11140-050) and 1× Penicillin/Streptomycin/Glutamine (Gibco, 10378-016). The cells were maintained weekly using 0.05% Trypsin (Gibco, 25300-054) and replated in 150 mm TC-Treated Culture Dishes (Corning, 430599).

The first day of the assay, the adherent cells were trypsinized, media was added to the loose cells and cells were mixed to a homogenous mixture. 0.5 ml of the homogenous mixture was counted on the Beckman Coulter® Vi-CELL™ XR. 50 frames of cells were counted and the number of viable cells was determined. The cells were then diluted to 0.25 million cells per ml, and centrifuged at 200 rcf for 5 minutes. The media was removed and the cells were reconstituted in fresh media for plating. The cells were plated at 20 μl per well on the PerkinElmer® FlexDrop PLUS in Low Volume 384 Well White Tissue Culture Plates (Corning, 3826) with a final cell density of 5K cells per well. The plates were incubated overnight at 37° Celsius, 5% CO₂.

On the second day, the compound plates were prepared, the cells were treated with compound and the p4EBP 1 reaction mix was added to the cell lysate. 384 well compound plates were prepared by Amgen's Sample Bank containing 1 μl of compound per well starting at 5 mM and diluted 1:2 across the row, resulting in a 22 well serial dilution. 39 μl of growth media was added to the compound plate in rows 1-22 using the PerkinElmer® FlexDrop PLUS resulting in a DMSO concentration of 2.5%. The control columns were added manually; 40 μl of 2.5% DMSO (Sigma, D4540-100 ml) in growth media was added to the plate for the high control and 40 μl of 50 μM of AMG2203766 with 2.5% DMSO was added to the plate as the low control. The cell plates and diluted compound plates were put onto the VELOCITY 1 1™ VPREP™ 384 ST where the compound plate was mixed and 5 μl of serially diluted compound or controls was added to the cell plate. The final concentration of the compounds was 25 μM serially diluted to 11.9 pM in 0.5% DMSO. The final high control had 0.5% DMSO and the low control concentration was 10 μM AMG2203766 in 0.5% DMSO. The cell plates were then incubated with compound for two hours at 37° Celsius, 5% CO₂. After two hours, the media in the cell plates was aspirated using the BioTek® ELx405HT plate washer removing the majority of media and compound without disturbing the adherent U87 cells. The following assay reagents are components of the SureFire Phospho-4EBP 1 (Thr37/Thr46) 50K Point Kit (TGR BioSciences, TGR4ES50K) and an IgG Detection Kit (PerkinElmer, 6760617R). 5 μl of 1× Lysis Buffer was added to each well using the PerkinElmer® FlexDrop PLUS. The plates were then incubated at room temperature on a shaker for ten minutes. The AlphaScreen reaction was prepared under low light conditions (subdued or green light) including p-4E-BP1 (Thr37/46) Reaction Buffer, Activation Buffer, Acceptor Beads and Donor Beads at a ratio of 60:10:1:1 respectively. The AlphaScreen reaction was added to the cell lysate at 6 μl per well using the PerkinElmer® FlexDrop PLUS. The plates were placed in a humid environment to reduce edge effects and incubated overnight at room temperature with restricted air flow in the dark.

On the final day of the experiment, the plates were read on the PerkinElmer® EnVision™ 2103 Multilable Reader using the standard AlphaScreen readout. The POC is calculated and the data is analyzed to report the IC₅₀ IP for p4EBP 1 at Thr37/Thr46.

### pAkt AlphaScreen

The pAkt AlphaScreen assay determines whether there is phosphorylation of Akt at Serine 473 by recruitment of a phosphospecific antibody. This assay was performed using U87 MG cells. The U87 growth media consists of MEM (Gibco, 51200-038) supplemented with 10% FBS (Gibco, 16140-071), 1× Non-Essential Amino Acids (Gibco, 11140-050) and 1× Penicillin/Streptomycin/Glutamine (Gibco, 10378-016). The cells were maintained weekly using 0.05% Trypsin (Gibco, 25300-054) and replated in 150 mm TC—Treated Culture Dishes (Corning, 430599).

The first day of the assay, the adherent cells were trypsinized, media was added to the loose cells and cells were mixed to a homogenous mixture. 0.5 ml of the homogenous mixture was counted on the Beckman Coulter® Vi-CELL™ XR. 50 frames of cells were counted and the number of viable cells was determined. The cells were then diluted to 0.25 million cells per ml, and centrifuged at 200 rcf for 5 minutes. The media was removed and the cells were reconstituted in fresh media for plating. The cells were plated at 20 μl per well on the PerkinElmer® FlexDrop PLUS in Low Volume 384 Well White Tissue Culture Plates (Corning, 3826) with a final cell density of 5K cells per well. The plates were incubated overnight at 37° Celsius, 5% CO₂.

On the second day, the compound plates were prepared, the cells were treated with compound and the pAkt reaction mix was added to the cell lysate. 384 well compound plates were prepared by Amgen's Sample Bank containing 1 μA of compound per well starting at 5 mM and diluted 1:2 across the row, resulting in a 22 well serial dilution. 39 μl of growth media was added to the compound plate in rows 1-22 using the PerkinElmer® FlexDrop PLUS resulting in a DMSO concentration of 2.5%. The control columns were added manually; 40 μl of 2.5% DMSO (Sigma, D4540-100 ml) in growth media was added to the plate for the high control and 40 μl of 50 μM of AMG2203766 with 2.5% DMSO was added to the plate as the low control. The cell plates and diluted compound plates were put onto the VELOCITY1 1™ VPREP™ 384 ST where the compound plate was mixed and 5 μl of serially diluted compound or controls was added to the cell plate. The final concentration of the compounds was 25 μM serially diluted to 11.9 pM in 0.5% DMSO. The final high control had 0.5% DMSO and the low control concentration was 10 μM AMG2203766 in 0.5% DMSO. The cell plates were then incubated with compound for two hours at 37° Celsius, 5% CO₂. After two hours, the media in the cell plates was aspirated using the BioTek® ELx405HT plate washer removing the majority of media and compound without disturbing the adherent U87 cells. The following assay reagents are components of the SureFire Akt (Ser 473) Phosphorylation 50K Point Kit (TGR BioSciences, TGRAS50K) and an IgG Detection Kit (PerkinElmer, 6760617R). 5 μl of 1× Lysis Buffer was added to each well using the PerkinElmer® FlexDrop PLUS. The plates were then incubated at room temperature on a shaker for ten minutes. The AlphaScreen reaction was prepared under low light conditions (subdued or green light) including p-Akt (Ser 473) Reaction Buffer, Dilution Buffer, Activation Buffer, Acceptor Beads and Donor Beads at a ratio of 40:20:10:1:1 respectively. The AlphaScreen reaction was added to the cell lysate at 6 μl per well using the PerkinElmer® FlexDrop PLUS. The plates were placed in a humid environment to reduce edge effects and incubated overnight at room temperature with restricted air flow in the dark.

On the final day of the experiment, the plates were read on the PerkinElmer® EnVision™ 2103 Multilable Reader using the standard AlphaScreen readout. The POC is calculated and the data is analyzed to report the IC₅₀ IP for pAkt at Serine 473.

TABLE 1

| | AA11016+++mTor_ | AA12047+++PI3Ka | AA11809+++pAKT_ |
|---|---|---|---|
| Cpd# | MA_IC₅₀_IP | _Alpha_IC₅₀_IP | U87_IC₅₀_IP |
| 1 | 0.000269 | 0.000646 | 0.00496 |
| 111 | 0.000765 | 0.05282 | 0.06992 |
| 2 | 0.001377 | | 0.22387 |
| 113 | 0.00166 | 0.058085 | 0.00668 |
| 115 | 0.004403 | 0.007312 | 0.131735 |
| 116 | 0.003207 | — | 0.029181 |
| 119 | 0.003569 | 0.053064 | 0.005891 |
| 122 | 0.007017 | 4.239355 | 0.041638 |
| 124 | 0.006647 | 0.094349 | 0.039437 |
| 126 | 0.007603 | 0.787953 | 0.028767 |
| 9 | 0.024348 | 0.485173 | 0.044624 |
| 16 | 0.062164 | 4.125903 | 1.426409 |
| 138 | 0.038717 | 1.394119 | 0.157543 |
| 141 | 0.055093 | 0.118705 | 0.202535 |
| 144 | 0.066588 | 7.255058 | 0.125215 |
| 24 | 0.075036 | 2.736503 | 0.444948 |
| 37 | 0.105293 | 1.335177 | 0.062791 |
| 151 | 0.123652 | 4.547887 | 0.08892 |
| 152 | 0.212761 | 4.854141 | 0.264003 |
| 156 | 0.183021 | 0.014555 | 0.054702 |
| 157 | 0.194491 | 31.80534 | 0.767003 |
| 159 | 0.241046 | 0.242973 | 0.491474 |
| 163 | 0.443711 | 17.19096 | 0.616867 |
| 170 | 1.170847 | 1.371565 | 1.683836 |
| 108 | 2.291394 | 0.469732 | 1.071766 |
| 60 | 7.818196 | | 2.356391 |
| 61 | 10.82164 | 3.832257 | 1.039572 |
| 62 | 11.08824 | | 2.014562 |
| 201 | — | 3.525334 | 0.652379 |
| 182 | — | 3.392344 | 1.181681 |
| 198 | — | 2.548212 | — |
| 90 | >50 | 6.455284 | 1.102809 |

### FORMULATION EXAMPLES

The following are representative pharmaceutical formulations containing a compound of Formula (I).

### Tablet Formulation

The following ingredients are mixed intimately and pressed into single scored tablets.

| | Quantity per |
|---|---|
| Ingredient | tablet mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule mg |
| compound of this invention | 200 |
| lactose spray dried | 148 |
| magnesium stearate | 2 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A compound of Formula (I): 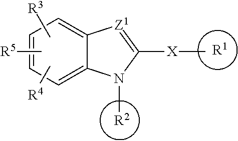
wherein:
Z¹ is —N— or —CH—;
X is —NR⁶— or —O— where R⁶ is hydrogen or alkyl;
R¹ is aryl, heteroaryl, cycloalkyl, fused cycloalkenyl, or heterocyclyl; each ring substituted with R^{a}, R^{b}, or R^{c} independently selected from hydrogen, alkyl, alkylthio, alkoxy, hydroxy, alkoxycarbonyl, carboxy, halo, haloalkyl, haloalkoxy, aminocarbonyl, aminosulfonyl, cycloalkyl, cycloalkylalkyl, acyl, cyano, aminoalkyl, hydroxyalkyl, optionally substituted heteroaryl, optionally substituted phenyl, amino, ureido, thioureido, monosubstituted, or disubstituted amino;
R² is:
(i) 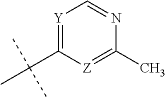
where Y and Z are independently —N═ or —C═; or
(ii) a five or six membered heterocyclyl ring;
each ring in (i) and (ii) is substituted with R^{d} and R^{e} where R^{d} and R^{e} are independently hydrogen, alkyl, alkoxy, halo, haloalkyl, haloalkoxy, amino, monosubstituted amino or disubstituted amino;
R³ and R⁴ are independently hydrogen, alkyl, halo, alkoxy, haloalkyl, hydroxyalkyl, alkoxyalkyl, cyano, carboxy, alkoxycarbonyl, aryl, heteroaryl, heterocyclyl, aralkyl, heteroaralkyl, heterocyclylalkyl, amino, monosubstituted amino, disubstituted amino, sulfonyl, acyl, hydroxyalkyloxy, alkoxyalkyloxy, aminoalkyl, aminoalkoxy, aryloxy, heteroaryloxy, heterocyclyloxy, aralkoxy, heteroaralkoxy, heterocyclylalkyloxy, aminosulfonyl, aminocarbonyl, or acylamino, where the aromatic or alicyclic ring in R³ and R⁴ is optionally substituted with R^{f}, R^{g} or R^{h} which are independently selected from alkyl, halo, haloalkyl, haloalkoxy, alkylthio, cyano, alkoxy, amino, monosubstituted amino, disubstituted amino, sulfonyl, acyl, carboxy, alkoxycarbonyl, hydroxyalkyl, alkoxyalkyl, aminoalkyl, hydroxyalkoxy, alkoxyalkoxy, aminoalkoxy, aminosulfonyl, aminocarbonyl, or acylamino; and
R⁵ is hydrogen, alkyl, halo, hydroxyl, alkoxy, haloalkyl, hydroxyalkyl, alkoxyalkyl, cyano, carboxy, alkoxycarbonyl, amino, alkylamino, or dialkylamino;
or a pharmaceutically acceptable salt thereof;
provided that:
(i) when Z¹ is —N═, R² is piperidin-4-yl, 4-methylpiperidin-1-yl, or 1-methylpiperidin-4-yl; X is —NH—, R³ is hydrogen, and R¹ is phenyl substituted at the 4-position with ethyl or —COR where R is methylamino, methoxy, methyl, or amino; 3,4,5-trimethyloxyphenyl, or 3,5-dimethoxyphenyl, then R⁴ is not —CON(CH₂CH₂CH(CH₃)₂)₂; or —CON(i-Bu)₂;
(ii) when Z¹ is —N═, R² is 6-chloro-5-methylpyrimidin-4-yl, 5-methyl-6-[4-diethylaminobutylamino]-pyrimidin-4-yl, or 6-amino-5-methylpyrimidin-4-yl, X is —NH—, R³ and R⁴ are hydrogen, then R¹ is not 6-methyl-3-(3-trifluoromethylphenylcarbonylamino)phenyl; and
(iii) when Z¹ is —N═, R² is tetrahydropyran-2-yl, X is —NH—, R³ and R⁴ are hydrogen, then R¹ is not piperidin-4-yl or 1-ethoxycarbonylpiperidin-4-yl; and
(iv) the compound is not 1-(4-amino-6-methyl-1,3,5-triazin-2-yl)-N-3-pyrrolidinyl-1H-benzimidazole-2-amine and 1-(4-amino-6-methyl-1,3,5-triazin-2-yl)-N-1H-imidazol-2-yl-1H-benzimidazole-2-amine.

2. The compound of claim 1 wherein Z¹ is —N— and X is —NR⁶; or a pharmaceutically acceptable salt thereof.

3. The compound of claim 1 wherein Z¹ is —N— and X is —NH—; or a pharmaceutically acceptable salt thereof.

4. The compound of claim 1 wherein Z¹ is —N— and X is —O—; or a pharmaceutically acceptable salt thereof.

5. The compound of claim 1 wherein R¹ is phenyl substituted with R^{a}, R^{b}, or R^{c}; or a pharmaceutically acceptable salt thereof.

6. The compound of claim 1 wherein R¹ is heteroaryl substituted R^{a}, R^{b}, or R^{c}; or a pharmaceutically acceptable salt thereof.

7. The compound of claim 1 wherein R¹ is pyrazolyl substituted R^{a}, R^{b}, or R^{c}; or a pharmaceutically acceptable salt thereof.

8. The compound of claim 1 wherein R¹ is phenyl substituted with R^{a}, R^{b} or R^{c} where R^{a} is hydrogen, R^{b} is hydrogen or hydroxy, and R^{c} is hydrogen, cyano, acyl, ureido, thioureido, alkoxycarbonyl, alkoxy, hydroxy, amino, cycloalkyl, carboxy, halo, aminocarbonyl, aminosulfonyl, alkyl, or monosubstituted amino (—NRR′ where R is hydrogen and R′ is hydrogen acyl, or sulfonyl) and R³, R⁴ and R⁵ are hydrogen; or a pharmaceutically acceptable salt thereof.

9. The compound of claim 1 wherein R¹ is 3,5-dihydroxyphenyl; 3-(—NHCONHCH₃)phenyl; 3-(—NHCONHCH₂CH₃)-phenyl; 3-(3-hydroxyphenyl-NHCONH—)phenyl; 3-hydroxy-5-methoxycarbonyl-phenyl; 3-hydroxy-4-methoxyphenyl; 3-(3-methoxyphenyl-NHCONH—)phenyl; 3-(4-methoxycarbonylphenyl-NHCONH—)phenyl; 4-carboxy-2-hydroxyphenyl; 4-hydroxyphenyl; 3-carboxyphenyl; 4-fluoro-3-hydroxyphenyl; 3-methoxyphenyl; 3-CONH₂phenyl; 3-hydroxy-4-methylphenyl; 4-(4-fluorophenylsulfonyl)-aminophenyl; 4-(4-methylphenylsulfonyl)-aminophenyl; 3-SO₂NH₂phenyl; phenyl; 4-methylsulfonylaminophenyl; 3-hydroxy-4-methoxycarbonylphenyl; 4-ethylcarbonylaminophenyl; 3-methylphenyl; 3-chlorophenyl; 3-fluorophenyl; 3-cyanophenyl; 4-acetylaminophenyl; 3-hydroxyphenyl; 3-methoxycarbonylphenyl; 3-hydroxy-4-acetylphenyl; 4-[4-methoxyphenylNHCSNH—]phenyl; 4-[3-methoxyphenylNHCSNH—]phenyl; 2,6-dichlorophenylsulfonyl-aminophenyl; 4-[ethylNHCSNH—]phenyl; 3-[3,5-dichlorophenylNHCSNH-]phenyl; 4-[2-trifluoromethylphenyl-sulfonylamino]]phenyl; 4-[—NHCONHCH₃]-phenyl; 4-[—NHCONHCH₂CH₃]phenyl; 4-[—NHCONHpyridin-4-yl]phenyl; 4-methylcarbonyl-aminophenyl; 4-cyclopropylcarbonylamino-phenyl; 3-F-4-hydroxyphenyl; 4-(ethylamino-carbonyl)phenyl; 4-(pyridin-4-ylaminocarbonyl)-phenyl; 3-methylcarbonylamino-phenyl; 4-(2,3-dihydro-1-benzofuran-5-ylcarbonylamino)phenyl; 4-aminophenyl; 4-(3-dimethylamino-phenyl-carbonylamino)phenyl; 4-(cyclopentylcarbonylamino)-phenyl; 4-(tert-butylcarbonylamino)-phenyl; 4-(2.1.3-benzothiadiazol-4-ylcarbonylamino)phenyl; 4-(2-isopropylaminocarbonyl)-phenyl; 4-(3,5-diClphenylNHCSNH—)phenyl; 4-(4-tert-butylphenylcarbonyl-amino)phenyl; 4-(cyclohexylcarbonyl-amino)phenyl; 4-(3-CF₃phenylcarbonyl-amino)phenyl; 4-(4-OCF₃phenylcarbonyl-amino)phenyl; or 4-(4-fluorophenylsulfonyl-amino)phenyl; or a pharmaceutically acceptable salt thereof.

10. The compound of claim 1 wherein R¹ is heteroaryl substituted with R^{a}, R^{b} or R^{c} where R^{a} and R^{b} are hydrogen, and R^{c} is hydrogen, cyano, acyl, ureido, thioureido, alkoxycarbonyl, alkoxy, hydroxy, amino, cycloalkyl, cycloalkylalkyl, optionally substituted heteroaryl, carboxy, halo, aminocarbonyl, alkyl, haloalkyl, or monosubstituted amino (—NRR′ where R is hydrogen and R′ is hydrogen acyl, or sulfonyl) and R³, R⁴ and R⁵ are hydrogen; or a pharmaceutically acceptable salt thereof.

11. The compound of claim 1 wherein R¹ is heteroaryl substituted with R^{a}, R^{b} or R^{c} where R^{a} and R^{b} are hydrogen, and R^{c} is hydrogen, cyano, acyl, alkoxycarbonyl, alkoxy, hydroxy, amino, cycloalkyl, cycloalkylalkyl, optionally substituted heteroaryl, halo, aminocarbonyl, alkyl, or haloalkyl and R³, R⁴ and R⁵ are hydrogen; or a pharmaceutically acceptable salt thereof.

12. The compound of claim 1 wherein R² is 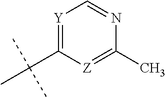
where Y and Z are independently —N═ or —C═ and is substituted with R^{d} where R^{d} is hydrogen, alkyl, alkoxy, halo, haloalkyl, haloalkoxy, amino, monosubstituted amino or disubstituted amino and R^{e} is hydrogen; or a pharmaceutically acceptable salt thereof.

13. The compound of claim 1 wherein R² is 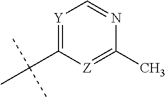
where Z is —N═ and is substituted with R^{d} where R^{d} is hydrogen, alkyl, alkoxy, halo, haloalkyl, haloalkoxy, amino, monosubstituted amino or disubstituted amino and R^{e} is hydrogen; or a pharmaceutically acceptable salt thereof.

14. The compound of claim 1 wherein R² is 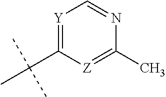
where Y and Z are —N═ and is substituted with R^{d} where R^{d} is hydrogen, alkyl, alkoxy, halo, haloalkyl, haloalkoxy, amino, monosubstituted amino or disubstituted amino and R^{e} is hydrogen; or a pharmaceutically acceptable salt thereof.

15. The compound of claim 1 wherein R² is 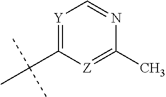
where Y and Z are —N═ and is substituted with R^{d} where R^{d} is monosubstituted amino and is at the 4-position of the triazin-2-yl ring and R^{e} is hydrogen; or a pharmaceutically acceptable salt thereof.

16. The compound of claim 1 wherein R² is 4-amino-6-methyl-1,3,5-triazin-2-yl; or a pharmaceutically acceptable salt thereof.

17. The compound of claim 1 selected from:
5-((1-(4-amino-6-methyl-1,3,5-triazin-2-yl)-1H-benzimidazol-2-yl)amino)-1,3-benzenediol;
1-(4-amino-6-methyl-1,3,5-triazin-2-yl)-2-((3-hydroxyphenyl)amino)-1H-benzimidazole-6-carboxylic acid;
3-((1-(4-amino-6-methyl-1,3,5-triazin-2-yl)-1H-benzimidazol-2-yl)amino)phenol;
1-(4-((1-(4-amino-6-methyl-1,3,5-triazin-2-yl)-1H-benzimidazol-2-yl)amino)phenyl)-3-methylurea;
1-(4-((1-(4-amino-6-methyl-1,3,5-triazin-2-yl)-1H-benzimidazol-2-yl)amino)phenyl)-3-ethylurea;
((4-(2-((3-hydroxyphenyl)amino)-1H-benzimidazol-1-yl)-6-methyl-1,3,5-triazin-2-yl)-amino)acetonitrile;
1-(4-((1-(4-amino-6-methyl-1,3,5-triazin-2-yl)-1H-benzimidazol-2-yl)amino)phenyl)-3-(3-hydroxyphenyl)urea;
methyl 3-((1-(4-amino-6-methyl-1,3,5-triazin-2-yl)-1H-benzimidazol-2-yl)amino)-5-hydroxybenzoate;
1-(4-amino-6-methyl-1,3,5-triazin-2-yl)-5,6-difluoro-N-1H-pyrazol-5-yl-1H-benzimidazol-2-amine;
1-(4-amino-6-methyl-1,3,5-triazin-2-yl)-N-1H-pyrazol-3-yl-1H-benzimidazol-2-amine;
Mixture of 1-(4-amino-6-methyl-1,3,5-triazin-2-yl)-5-fluoro-N-1H-pyrazol-5-yl-1H-benzimidazol-2-amine and 1-(4-amino-6-methyl-1,3,5-triazin-2-yl)-6-fluoro-N-1H-pyrazol-5-yl-1H-benzimidazol-2-amine;
5-((1-(4-amino-6-methyl-1,3,5-triazin-2-yl)-1H-benzimidazol-2-yl)amino)-2-methoxyphenol;
N-(1-(4-amino-6-methyl-1,3,5-triazin-2-yl)-1H-benzimidazol-2-yl)-1H-indazol-6-amine;
1-(4-amino-6-methyl-1,3,5-triazin-2-yl)-N-1H-indol-6-yl-1H-benzimidazol-2-amine;
1-(4-amino-6-methyl-1,3,5-triazin-2-yl)-N-(3-cyclopropyl-1H-pyrazol-5-yl)-1H-benzimidazol-2-amine;
1-(4-((1-(4-amino-6-methyl-1,3,5-triazin-2-yl)-1H-benzimidazol-2-yl)amino)phenyl)-3-(3-methoxyphenyl)urea;
methyl 4-(((4-((1-(4-amino-6-methyl-1,3,5-triazin-2-yl)-1H-benzimidazol-2-yl)amino)-phenyl)carbamoyl)amino)benzoate;
3-((1-(4-amino-6-methyl-1,3,5-triazin-2-yl)-5,6-dimethyl-1H-benzimidazol-2-yl)-amino)phenol;
N˜5˜-(1-(4-amino-6-methyl-1,3,5-triazin-2-yl)-1H-benzimidazol-2-yl)-2,5-pyridine-diamine;
N-(1-(4-amino-6-methyl-1,3,5-triazin-2-yl)-1H-benzimidazol-2-yl)-1H-indazol-3-amine;
4-((1-(4-amino-6-methyl-1,3,5-triazin-2-yl)-1H-benzimidazol-2-yl)amino)-2-hydroxybenzoic acid;
1-(4-amino-6-methyl-1,3,5-triazin-2-yl)-N-1H-indol-4-yl-1H-benzimidazol-2-amine;
N-(1-(4-amino-6-methyl-1,3,5-triazin-2-yl)-1H-benzimidazol-2-yl)-4-fluoro-1H-indazol-3-amine;
4-((1-(4-amino-6-methyl-1,3,5-triazin-2-yl)-1H-benzimidazol-2-yl)amino)phenol;
3-((1-(4-amino-6-methyl-1,3,5-triazin-2-yl)-1H-benzimidazol-2-yl)amino)benzoic acid;
mixture of 1-(4-amino-6-methyl-1,3,5-triazin-2-yl)-5-bromo-N-1H-pyrazol-3-yl-1H-benzimidazol-2-amine and 1-(4-amino-6-methyl-1,3,5-triazin-2-yl)-6-bromo-N-1H-pyrazol-3-yl-1H-benzimidazol-2-amine;
1-(4-amino-6-methyl-1,3,5-triazin-2-yl)-5,6-dimethyl-N-1H-pyrazol-5-yl-1H-benzimidazol-2-amine;
1-(4-amino-6-methyl-1,3,5-triazin-2-yl)-N-3-pyridinyl-1H-benzimidazol-2-amine;
1-(4-amino-6-methyl-1,3,5-triazin-2-yl)-N-3-isoxazolyl-1H-benzimidazol-2-amine;
1-(4-amino-6-methyl-1,3,5-triazin-2-yl)-N-1H-pyrazol-4-yl-1H-benzimidazol-2-amine;
5-((1-(4-amino-6-methyl-1,3,5-triazin-2-yl)-1H-benzimidazol-2-yl)amino)-2-fluorophenol;
methyl 1-(4-amino-6-methyl-1,3,5-triazin-2-yl)-2-((3-methoxyphenyl)amino)-1H-benzimidazole-5-carboxylate;
3-((1-(4-amino-6-methyl-1,3,5-triazin-2-yl)-1H-benzimidazol-2-yl)amino)benzamide;
5-((1-(4-amino-6-methyl-1,3,5-triazin-2-yl)-1H-benzimidazol-2-yl)amino)-2-methylphenol;
(1-(4-amino-6-methyl-1,3,5-triazin-2-yl)-2-((3-methoxyphenyl)amino)-1H-benzimidazol-5-yl)methanol;
N-(4-((1-(4-amino-6-methyl-1,3,5-triazin-2-yl)-1H-benzimidazol-2-yl)amino)phenyl)-4-fluorobenzenesulfonamide;
N-(1-(4-amino-6-methyl-1,3,5-triazin-2-yl)-1H-benzimidazol-2-yl)-2,6-pyridine-diamine;
6-((1-(4-amino-6-methyl-1,3,5-triazin-2-yl)-1H-benzimidazol-2-yl)amino)-2,3-dihydro-1H-inden-1-one;
1-(4-amino-6-methyl-1,3,5-triazin-2-yl)-N-(1-methyl-1H-pyrazol-3-yl)-1H-benzimidazol-2-amine;
1-(4-amino-6-methyl-1,3,5-triazin-2-yl)-N-(3-methoxyphenyl)-1H-benzimidazol-2-amine;
1-(4-amino-6-methyl-1,3,5-triazin-2-yl)-N-(3-cyclopropyl-1H-pyrazol-5-yl)-5,6-dimethyl-1H-benzimidazol-2-amine;
N-(4-((1-(4-amino-6-methyl-1,3,5-triazin-2-yl)-1H-benzimidazol-2-yl)amino)phenyl)-4-methylbenzenesulfonamide;
1-(4-amino-6-methyl-1,3,5-triazin-2-yl)-N-(3-methyl-5-isothiazolyl)-1H-benzimidazol-2-amine;
3-((1-(4-amino-6-methyl-1,3,5-triazin-2-yl)-1H-benzimidazol-2-yl)amino)-benzenesulfonamide;
1-(4-amino-6-methyl-1,3,5-triazin-2-yl)-N-phenyl-1H-benzimidazol-2-amine;
N-(4-((1-(4-amino-6-methyl-1,3,5-triazin-2-yl)-1H-benzimidazol-2-yl)amino)phenyl)-methanesulfonamide;
(1-(4-amino-6-methyl-1,3,5-triazin-2-yl)-2-(3-methoxyphenylamino)-1H-benzo[d]-imidazol-6-yl)methanol;
methyl 4-((1-(4-amino-6-methyl-1,3,5-triazin-2-yl)-1H-benzimidazol-2-yl)amino)-2-hydroxybenzoate;
1-(4-amino-6-methyl-1,3,5-triazin-2-yl)-N-(5-methyl-1H-pyrazol-3-yl)-1H-benzimidazol-2-amine;
1-(4-methyl-6-(methylamino)-1,3,5-triazin-2-yl)-N-1H-pyrazol-3-yl-1H-benzimidazol-2-amine;
1-(4-amino-6-methyl-1,3,5-triazin-2-yl)-N-(3-(2-furanyl)-1H-pyrazol-5-yl)-1H-benzimidazol-2-amine;
N-(4-((1-(4-amino-6-methyl-1,3,5-triazin-2-yl)-1H-benzimidazol-2-yl)amino)phenyl)-propanamide;
3-((1-(2-methyl-4-pyrimidinyl)-1H-benzimidazol-2-yl)amino)phenol;
1-(4-amino-6-methyl-1,3,5-triazin-2-yl)-N-(3-methylphenyl)-1H-benzimidazol-2-amine;
1-(4-amino-6-methyl-1,3,5-triazin-2-yl)-5,6-dimethyl-N-(3-methyl-1H-pyrazol-5-yl)-1H-benzimidazol-2-amine;
1-(4-amino-6-methyl-1,3,5-triazin-2-yl)-N-(3-(1-methylethyl)-1H-pyrazol-5-yl)-1H-benzimidazol-2-amine;
1-(4-amino-6-methyl-1,3,5-triazin-2-yl)-N-(3-chlorophenyl)-1H-benzimidazol-2-amine;
1-(4-amino-6-methyl-1,3,5-triazin-2-yl)-N-(3-fluorophenyl)-1H-benzimidazol-2-amine;
1-(4-amino-6-methyl-1,3,5-triazin-2-yl)-N-(3-(2-thiophenyl)-1H-pyrazol-5-yl)-1H-benzimidazol-2-amine;
1-(4-amino-6-methyl-1,3,5-triazin-2-yl)-N-((3RS)-tetrahydro-3-furanyl)-1H-benzimidazol-2-amine;
1-(4-amino-6-methyl-1,3,5-triazin-2-yl)-N-cyclopropyl-1H-benzimidazol-2-amine;
1-(4-amino-6-methyl-1,3,5-triazin-2-yl)-N-1H-1,2,4-triazol-3-yl-1H-benzimidazol-2-amine;
5-((1-(4-amino-6-methyl-1,3,5-triazin-2-yl)-1H-benzimidazol-2-yl)amino)-3-pyridinol;
1-(4-amino-6-methyl-1,3,5-triazin-2-yl)-N-(5-methyl-4H-1,2,4-triazol-3-yl)-1H-benzimidazol-2-amine;
1-(4-amino-6-methyl-1,3,5-triazin-2-yl)-N-(tetrahydro-2H-pyran-4-yl)-1H-benzimidazol-2-amine;
N-(1-(4-amino-6-methyl-1,3,5-triazin-2-yl)-1H-benzimidazol-2-yl)-1H-pyrazolo[3,4-b]-pyridin-3-amine;
N-(1-(4-amino-6-methyl-1,3,5-triazin-2-yl)-1H-benzimidazol-2-yl)-1-methyl-1H-indazol-3-amine;
5-((1-(4-amino-6-methyl-1,3,5-triazin-2-yl)-1H-benzimidazol-2-yl)amino)-2-pyridinol;
3-((1-(4-amino-6-methyl-1,3,5-triazin-2-yl)-1H-benzimidazol-2-yl)amino)benzonitrile;
N-(4-((1-(4-amino-6-methyl-1,3,5-triazin-2-yl)-1H-benzimidazol-2-yl)amino)-phenyl)acetamide;
1-(4-amino-6-methyl-1,3,5-triazin-2-yl)-N-(2,3-dihydro-1H-inden-2-yl)-1H-benzimidazol-2-amine;
5-((1-(4-amino-6-methyl-1,3,5-triazin-2-yl)-1H-benzimidazol-2-yl)amino)-1H-pyrazol-3-ol;
N-(1-(4-amino-6-methyl-1,3,5-triazin-2-yl)-1H-benzimidazol-2-yl)-1-(cyclopropyl-methyl)-1H-indazol-3-amine;
1-(4-amino-6-methyl-1,3,5-triazin-2-yl)-N-(2,3-dihydro-1H-inden-1-yl)-1H-benzimidazol-2-amine;
3-((1-(4-amino-6-methyl-1,3,5-triazin-2-yl)-5,6-dichloro-1H-benzimidazol-2-yl)-amino)phenol;
5-((1-(4-amino-6-methyl-1,3,5-triazin-2-yl)-1H-benzimidazol-2-yl)amino)-1H-pyrazole-4-carbonitrile;
1-(4-amino-6-methyl-1,3,5-triazin-2-yl)-N-(1-methyl-1H-pyrazol-5-yl)-1H-benzimidazol-2-amine;
ethyl 5-((1-(4-amino-6-methyl-1,3,5-triazin-2-yl)-1H-benzimidazol-2-yl)amino)-1H-pyrazole-4-carboxylate;
methyl 3-((1-(4-amino-6-methyl-1,3,5-triazin-2-yl)-1H-benzimidazol-2-yl)amino)-benzoate;
4-(2-((3-amino-1H-pyrazol-5-yl)oxy)-1H-benzimidazol-1-yl)-6-methyl-1,3,5-triazin-2-amine;
4-(2-(3-methoxyphenoxy)-1H-benzimidazol-1-yl)-6-methyl-1,3,5-triazin-2-amine;
1-(4-amino-6-methyl-1,3,5-triazin-2-yl)-N-1H-imidazol-2-yl-1H-benzimidazol-2-amine;
4-((1-(4-amino-6-methyl-1,3,5-triazin-2-yl)-1H-benzimidazol-2-yl)amino)-2,3-dihydro-1H-isoindol-1-one;
1-(4-amino-6-methyl-1,3,5-triazin-2-yl)-N-1,3-thiazol-2-yl-1H-benzimidazol-2-amine;
1-(4-((1-(4-amino-6-methyl-1,3,5-triazin-2-yl)-1H-benzimidazol-2-yl)amino)-2-hydroxy-phenyl)ethanone;
methyl 1-((1-(4-amino-6-methyl-1,3,5-triazin-2-yl)-1H-benzimidazol-2-yl)amino)-cyclopropanecarboxylate;
1-(4-amino-6-methyl-1,3,5-triazin-2-yl)-N-(3-tert-butyl-1H-pyrazol-5-yl)-1H-benzimidazol-2-amine;
N-(1-(4-amino-6-methyl-1,3,5-triazin-2-yl)-1H-benzimidazol-2-yl)-5-(trifluoromethyl)-1H-indazol-3-amine;
1-(4-amino-6-methyl-1,3,5-triazin-2-yl)-N-(1-(4-chlorophenyl)cyclopropyl)-1H-benzimidazol-2-amine;
methyl 1-(4-amino-6-methyl-1,3,5-triazin-2-yl)-2-((3-methoxyphenyl)amino)-1H-benzimidazole-6-carboxylate;
1-(4-amino-6-methyl-1,3,5-triazin-2-yl)-N-(3-cyclohexyl-1H-pyrazol-5-yl)-1H-benzimidazol-2-amine;
3-((1-(6-amino-2-methyl-4-pyrimidinyl)-1H-benzimidazol-2-yl)amino)phenol;
3-((1-(6-pyrimidin-5-ylamino-2-methyl-4-pyrimidinyl)-1H-benzimidazol-2-yl)-amino)phenol
1-(6-amino-2-methyl-4-pyrimidinyl)-N-1H-pyrazol-3-yl-1H-benzimidazol-2-amine;
3-((1-(6-pyrimidin-2-ylamino-2-methyl-4-pyrimidinyl)-1H-benzimidazol-2-yl)-amino)phenol
1-(6-amino-2-methyl-4-pyrimidinyl)-N-(3-cyclopropyl-1H-pyrazol-5-yl)-1H-benzimidazol-2-amine;
3-((1-(2-amino-6-methyl-4-pyrimidinyl)-1H-benzimidazol-2-yl)oxy)phenol;
4-(2-(3-methoxyphenoxy)-1H-benzimidazol-1-yl)-6-methyl-2-pyrimidinamine;
1-(4-amino-6-methyl-1,3,5-triazin-2-yl)-5,6-dichloro-N-1H-pyrazol-5-yl-1H-benzimidazol-2-amine;
1-(4-((1-(4-amino-6-methyl-1,3,5-triazin-2-yl)-1H-benzimidazol-2-yl)amino)phenyl)-3-(4-methoxyphenyl)thiourea;
1-(4-((1-(4-amino-6-methyl-1,3,5-triazin-2-yl)-1H-benzimidazol-2-yl)amino)phenyl)-3-(3-methoxyphenyl)thiourea;
1-(4-amino-6-methyl-1,3,5-triazin-2-yl)-4-methyl-N-1H-pyrazol-5-yl-1H-benz-imidazol-2-amine;
N-(4-((1-(4-amino-6-methyl-1,3,5-triazin-2-yl)-1H-benzimidazol-2-yl)amino)phenyl)-2,6-dichlorobenzenesulfonamide;
((4-methyl-6-(2-(1H-pyrazol-3-ylamino)-1H-benzimidazol-1-yl)-1,3,5-triazin-2-yl)-amino)acetonitrile;
N-3-isoxazolyl-1-(4-methyl-6-(methylamino)-1,3,5-triazin-2-yl)-1H-benzimidazol-2-amine;
((4-(2-(3-isoxazolylamino)-1H-benzimidazol-1-yl)-6-methyl-1,3,5-triazin-2-yl)amino)-acetonitrile;
1-(4-((1-(4-amino-6-methyl-1,3,5-triazin-2-yl)-1H-benzimidazol-2-yl)amino)phenyl)-3-ethylthiourea;
1-(4-((1-(4-amino-6-methyl-1,3,5-triazin-2-yl)-1H-benzimidazol-2-yl)amino)phenyl)-3-(3,5-dichlorophenyl)thiourea; and
N-(4-((1-(4-amino-6-methyl-1,3,5-triazin-2-yl)-1H-benzimidazol-2-yl)amino)phenyl)-2-(trifluoromethyl)benzene sulfonamide.
1-(4-((1-(4-((cyanomethyl)amino)-6-methyl-1,3,5-triazin-2-yl)-1H-benzimidazol-2-yl)-amino)phenyl)-3-methylurea;
1-(4-((1-(4-((cyanomethyl)amino)-6-methyl-1,3,5-triazin-2-yl)-1H-benzimidazol-2-yl)-amino)phenyl)-3-ethylurea;
1-(4-methyl-6-(methylamino)-1,3,5-triazin-2-yl)-N-1H-pyrazol-3-yl-6-(5-pyrimidinyl)-1H-benzimidazol-2-amine;
1-(4-((1-(4-amino-6-methyl-1,3,5-triazin-2-yl)-1H-benzimidazol-2-yl)amino)phenyl)-3-(4-pyridinyl)urea;
mixture of 1-(4-methyl-6-(methylamino)-1,3,5-triazin-2-yl)-5-(2-methyl-4-pyridinyl)-N-1H-pyrazol-3-yl-1H-benzimidazol-2-amine and 1-(4-methyl-6-(methylamino)-1,3,5-triazin-2-yl)-6-(2-methyl-4-pyridinyl)-N-1H-pyrazol-3-yl-1H-benzimidazol-2-amine;
mixture of 1-(4-amino-6-methyl-1,3,5-triazin-2-yl)-5-(1H-pyrazol-4-yl)-N-1H-pyrazol-5-yl-1H-benzimidazol-2-amine and 1-(4-amino-6-methyl-1,3,5-triazin-2-yl)-6-(1H-pyrazol-4-yl)-N-1H-pyrazol-5-yl-1H-benzimidazol-2-amine;
mixture of 1-(4-methyl-6-(methylamino)-1,3,5-triazin-2-yl)-N-1H-pyrazol-3-yl-5-(5-pyrimidinyl)-1H-benzimidazol-2-amine and 1-(4-methyl-6-(methylamino)-1,3,5-triazin-2-yl)-N-1H-pyrazol-3-yl-6-(5-pyrimidinyl)-1H-benzimidazol-2-amine;
N-(4-((1-(4-methyl-6-(methylamino)-1,3,5-triazin-2-yl)-1H-benzimidazol-2-yl)-amino)phenyl)acetamide;
mixture of 1-(4-amino-6-methyl-1,3,5-triazin-2-yl)-N-1H-pyrazol-5-yl-5-(4-pyridinyl)-1H-benzimidazol-2-amine; and 1-(4-amino-6-methyl-1,3,5-triazin-2-yl)-N-1H-pyrazol-5-yl-6-(4-pyridinyl)-1H-benzimidazol-2-amine;
1-(4-amino-6-methyl-1,3,5-triazin-2-yl)-N-1H-pyrazol-5-yl-6-(3-pyridinyl)-1H-benzimidazol-2-amine;
mixture of 1-(4-methyl-6-(methylamino)-1,3,5-triazin-2-yl)-N-1H-pyrazol-3-yl-5-(1,2,3,6-tetrahydro-4-pyridinyl)-1H-benzimidazol-2-amine; and 1-(4-methyl-6-(methylamino)-1,3,5-triazin-2-yl)-N-1H-pyrazol-3-yl-6-(1,2,3,6-tetrahydro-4-pyridinyl)-1H-benzimidazol-2-amine;
1-(4-amino-6-methyl-1,3,5-triazin-2-yl)-N-1H-pyrazol-5-yl-6-(4-pyridinyl)-1H-benzimidazol-2-amine;
mixture of (5-(1-(4-amino-6-methyl-1,3,5-triazin-2-yl)-2-(1H-pyrazol-5-ylamino)-1H-benzimidazol-6-yl)-2-methoxyphenyl)methanol; and (5-(1-(4-amino-6-methyl-1,3,5-triazin-2-yl)-2-(1H-pyrazol-5-ylamino)-1H-benzimidazol-5-yl)-2-methoxyphenyl)methanol;
mixture of N-methyl-4-(1-(4-methyl-6-(methylamino)-1,3,5-triazin-2-yl)-2-(1H-pyrazol-3-ylamino)-1H-benzimidazol-5-yl)benzamide; and N-methyl-4-(1-(4-methyl-6-(methylamino)-1,3,5-triazin-2-yl)-2-(1H-pyrazol-3-ylamino)-1H-benzimidazol-6-yl)benzamide;
mixture of 2-fluoro-N-methyl-4-(1-(4-methyl-6-(methylamino)-1,3,5-triazin-2-yl)-2-(1H-pyrazol-3-ylamino)-1H-benzimidazol-5-yl)benzamide; and 2-fluoro-N-methyl-4-(1-(4-methyl-6-(methylamino)-1,3,5-triazin-2-yl)-2-(1H-pyrazol-3-ylamino)-1H-benzimidazol-6-yl)benzamide;
N-(4-((1-(4-((cyanomethyl)amino)-6-methyl-1,3,5-triazin-2-yl)-1H-benzimidazol-2-yl)amino)phenyl)acetamide;
mixture of ((4-(5-(3-(hydroxymethyl)-4-methoxyphenyl)-2-(1H-pyrazol-3-ylamino)-1H-benzimidazol-1-yl)-6-methyl-1,3,5-triazin-2-yl)amino)acetonitrile; and ((4-(6-(3-(hydroxymethyl)-4-methoxyphenyl)-2-(1H-pyrazol-3-ylamino)-1H-benzimidazol-1-yl)-6-methyl-1,3,5-triazin-2-yl)amino)acetonitrile;
mixture of N-(4-(1-(4-methyl-6-(methylamino)-1,3,5-triazin-2-yl)-2-(1H-pyrazol-3-ylamino)-1H-benzimidazol-5-yl)phenyl)acetamide; and N-(4-(1-(4-methyl-6-(methylamino)-1,3,5-triazin-2-yl)-2-(1H-pyrazol-3-ylamino)-1H-benzimidazol-6-yl)phenyl)acetamide;
mixture of 1-(4-methyl-6-(methylamino)-1,3,5-triazin-2-yl)-N-1H-pyrazol-3-yl-5-(3-pyridinyl)-1H-benzimidazol-2-amine; and 1-(4-methyl-6-(methylamino)-1,3,5-triazin-2-yl)-N-1H-pyrazol-3-yl-6-(3-pyridinyl)-1H-benzimidazol-2-amine;
mixture of 1-(4-amino-6-methyl-1,3,5-triazin-2-yl)-2-(1H-pyrazol-5-ylamino)-1H-benzimidazol-5-ol; and -(4-amino-6-methyl-1,3,5-triazin-2-yl)-2-(1H-pyrazol-5-ylamino)-1H-benzimidazol-6-ol;
mixture of ((4-methyl-6-(2-(1H-pyrazol-3-ylamino)-5-(4-pyridinyl)-1H-benzimidazol-1-yl)-1,3,5-triazin-2-yl)amino)acetonitrile; and ((4-methyl-6-(2-(1H-pyrazol-3-ylamino)-6-(4-pyridinyl)-1H-benzimidazol-1-yl)-1,3,5-triazin-2-yl)amino)acetonitrile;
mixture of 1-(4-amino-6-methyl-1,3,5-triazin-2-yl)-5-methoxy-N-1H-pyrazol-5-yl-1H-benzimidazol-2-amine; and 1-(4-amino-6-methyl-1,3,5-triazin-2-yl)-6-methoxy-N-1H-pyrazol-5-yl-1H-benzimidazol-2-amine;
mixture of (2-methoxy-5-(1-(4-methyl-6-(methylamino)-1,3,5-triazin-2-yl)-2-(1H-pyrazol-3-ylamino)-1H-benzimidazol-5-yl)phenyl)methanol; and (2-methoxy-5-(1-(4-methyl-6-(methylamino)-1,3,5-triazin-2-yl)-2-(1H-pyrazol-3-ylamino)-1H-benzimidazol-6-yl)phenyl)methanol;
N˜5˜-(1-(4-amino-6-methyl-1,3,5-triazin-2-yl)-1H-benzimidazol-2-yl)-2,5-pyrimidinediamine;
((4-(5,6-dimethyl-2-(1H-pyrazol-3-ylamino)-1H-benzimidazol-1-yl)-6-methyl-1,3,5-triazin-2-yl)amino)acetonitrile;
4-((1-(4-amino-6-methyl-1,3,5-triazin-2-yl)-1H-benzimidazol-2-yl)amino)-N-methylbenzamide;
5,6-dimethyl-1-(4-methyl-6-(methylamino)-1,3,5-triazin-2-yl)-N-1H-pyrazol-3-yl-1H-benzimidazol-2-amine;
mixture of 1-(4-methyl-6-(methylamino)-1,3,5-triazin-2-yl)-N-1H-pyrazol-3-yl-5-(4-pyridinyl)-1H-benzimidazol-2-amine; and 1-(4-methyl-6-(methylamino)-1,3,5-triazin-2-yl)-N-1H-pyrazol-3-yl-6-(4-pyridinyl)-1H-benzimidazol-2-amine;
mixture of (5-(1-(4-amino-6-methyl-1,3,5-triazin-2-yl)-2-(1H-pyrazol-5-ylamino)-1H-benzimidazol-5-yl)-2-methoxyphenyl)methanol;
N-(4-((1-(4-amino-6-methyl-1,3,5-triazin-2-yl)-1H-benzimidazol-2-yl)amino)phenyl)cyclopropanecarboxamide;
5-(4-methoxyphenyl)-1-(4-methyl-6-(methylamino)-1,3,5-triazin-2-yl)-N-1H-pyrazol-3-yl-1H-benzimidazol-2-amine;
3-((4-methyl-6-(2-(1H-pyrazol-3-ylamino)-1H-benzimidazol-1-yl)-1,3,5-triazin-2-yl)amino)propanenitrile;
2-((4-methyl-6-(2-(1H-pyrazol-3-ylamino)-1H-benzimidazol-1-yl)-1,3,5-triazin-2-yl)amino)ethanol;
5,6-difluoro-1-(4-methyl-6-(methylamino)-1,3,5-triazin-2-yl)-N-1H-pyrazol-5-yl-1H-benzimidazol-2-amine;
1-(4-amino-6-methyl-1,3,5-triazin-2-yl)-N-1H-pyrazol-5-yl-5-(4-pyridinyl)-1H-benzimidazol-2-amine;
mixture of 5-(4-fluorophenyl)-1-(4-methyl-6-(methylamino)-1,3,5-triazin-2-yl)-N-1H-pyrazol-3-yl-1H-benzimidazol-2-amine; and 6-(4-fluorophenyl)-1-(4-methyl-6-(methylamino)-1,3,5-triazin-2-yl)-N-1H-pyrazol-3-yl-1H-benzimidazol-2-amine;
4-((1-(4-amino-6-methyl-1,3,5-triazin-2-yl)-1H-benzimidazol-2-yl)amino)-2-fluorophenol;
5-((1-(4-amino-6-methyl-1,3,5-triazin-2-yl)-1H-benzimidazol-2-yl)amino)-1,3-dihydro-2H-benzimidazol-2-one;
1-(4-methyl-6-(methylamino)-1,3,5-triazin-2-yl)-N-1H-pyrazol-3-yl-5-(5-pyrimidinyl)-1H-benzimidazol-2-amine;
1-(4-((2-methoxyethyl)amino)-6-methyl-1,3,5-triazin-2-yl)-N-1H-pyrazol-3-yl-1H-benzimidazol-2-amine;
1-(4-(cyclopropylamino)-6-methyl-1,3,5-triazin-2-yl)-N-1H-pyrazol-5-yl-1H-benzimidazol-2-amine;
1-(4-amino-6-methyl-1,3,5-triazin-2-yl)-N-(6-methoxy-3-pyridinyl)-1H-benzimidazol-2-amine;
4-((1-(4-amino-6-methyl-1,3,5-triazin-2-yl)-1H-benzimidazol-2-yl)amino)-N-ethyl-benzamide;
1-(4-(benzylamino)-6-methyl-1,3,5-triazin-2-yl)-N-1H-pyrazol-3-yl-1H-benzimidazol-2-amine;
N-(1-(4-amino-6-methyl-1,3,5-triazin-2-yl)-1H-benzimidazol-2-yl)-1H-indazol-4-amine;
1-(4-methyl-6-((2-(1-piperazinyl)ethyl)amino)-1,3,5-triazin-2-yl)-N-1H-pyrazol-3-yl-1H-benzimidazol-2-amine;
1-(4-methyl-6-((2-(4-morpholinyl)ethyl)amino)-1,3,5-triazin-2-yl)-N-1H-pyrazol-3-yl-1H-benzimidazol-2-amine;
4-((1-(4-amino-6-methyl-1,3,5-triazin-2-yl)-1H-benzimidazol-2-yl)amino)-N-4-pyridinylbenzamide;
1-(4-(ethylamino)-6-methyl-1,3,5-triazin-2-yl)-N-1H-pyrazol-3-yl-1H-benzimidazol-2-amine;
N-(3-((1-(4-amino-6-methyl-1,3,5-triazin-2-yl)-1H-benzimidazol-2-yl)amino)-phenyl)acetamide;
1-(4-methyl-6-((tetrahydro-2H-pyran-4-ylmethyl)amino)-1,3,5-triazin-2-yl)-N-1H-pyrazol-3-yl-1H-benzimidazol-2-amine;
1-(4-methyl-6-(tetrahydro-2H-pyran-4-ylamino)-1,3,5-triazin-2-yl)-N-1H-pyrazol-3-yl-1H-benzimidazol-2-amine;
N-(4-((1-(4-amino-6-methyl-1,3,5-triazin-2-yl)-1H-benzimidazol-2-yl)amino)phenyl)-2,3-dihydro-1-benzofuran-5-carboxamide;
1-(4-methyl-6-((1-methylethyl)amino)-1,3,5-triazin-2-yl)-N-1H-pyrazol-3-yl-1H-benzimidazol-2-amine;
1-(4-((1H-imidazol-2-ylmethyl)amino)-6-methyl-1,3,5-triazin-2-yl)-N-1H-pyrazol-3-yl-1H-benzimidazol-2-amine;
N-(1-(4-amino-6-methyl-1,3,5-triazin-2-yl)-1H-benzimidazol-2-yl)-1,4-benzene-diamine;
N-(4-((1-(4-amino-6-methyl-1,3,5-triazin-2-yl)-1H-benzimidazol-2-yl)amino)phenyl)-3-(dimethylamino)benz amide;
1-(4-amino-6-methyl-1,3,5-triazin-2-yl)-N-(1-methyl-1H-pyrazol-4-yl)-1H-benzimidazol-2-amine;
N-(4-((1-(4-amino-6-methyl-1,3,5-triazin-2-yl)-1H-benzimidazol-2-yl)amino)phenyl)-cyclopentanecarboxamide;
N-(4-((1-(4-amino-6-methyl-1,3,5-triazin-2-yl)-1H-benzimidazol-2-yl)amino)phenyl)-2,2-dimethylpropanamide;
1-(4-((cyclopropylmethyl)amino)-6-methyl-1,3,5-triazin-2-yl)-N-1H-pyrazol-3-yl-1H-benzimidazol-2-amine;
1-(4-amino-6-methyl-1,3,5-triazin-2-yl)-N-2-pyrimidinyl-1H-benzimidazol-2-amine;
1-(4-methyl-1,3,5-triazin-2-yl)-N-1H-pyrazol-3-yl-1H-benzimidazol-2-amine;
N-(4-((1-(4-amino-6-methyl-1,3,5-triazin-2-yl)-1H-benzimidazol-2-yl)amino)phenyl)-2,1,3-benzothiadiazole-4-carboxamide;
1-(4-methyl-6-(2-pyrimidinylamino)-1,3,5-triazin-2-yl)-N-1H-pyrazol-3-yl-1H-benz-imidazol-2-amine;
4-((1-(4-amino-6-methyl-1,3,5-triazin-2-yl)-1H-benzimidazol-2-yl)amino)-N-(1-methylethyl)benzamide;
1-(4-amino-6-methyl-1,3,5-triazin-2-yl)-N-(3-methylphenyl)-1H-benzimidazol-2-amine;
1-(4-amino-6-methyl-1,3,5-triazin-2-yl)-N-(1-(2,2,2-trifluoroethyl)-1H-pyrazol-3-yl)-1H-benzimidazol-2-amine;
1-(4-amino-6-methyl-1,3,5-triazin-2-yl)-N-4-pyrimidinyl-1H-benzimidazol-2-amine;
5-((1-(4-amino-6-methyl-1,3,5-triazin-2-yl)-1H-benzimidazol-2-yl)amino)-N-methyl-1H-pyrazole-3-carboxamide;
1-(4-((1-(4-((cyanomethyl)amino)-6-methyl-1,3,5-triazin-2-yl)-1H-benzimidazol-2-yl)amino)phenyl)-3-(3,5-dichlorophenyl)thiourea;
1-(4-amino-6-methyl-1,3,5-triazin-2-yl)-N-2-pyrazinyl-1H-benzimidazol-2-amine;
3-((1-(4-amino-6-methyl-1,3,5-triazin-2-yl)-1H-benzimidazol-2-yl)amino)benzonitrile;
1-(4-amino-6-methyl-1,3,5-triazin-2-yl)-N-4-pyridazinyl-1H-benzimidazol-2-amine;
4-((1-(4-amino-6-methyl-1,3,5-triazin-2-yl)-1H-benzimidazol-2-yl)amino)-2-pyrrolidinone;
5-((1-(4-amino-6-methyl-1,3,5-triazin-2-yl)-1H-benzimidazol-2-yl)amino)-N-cyclopropyl-1H-pyrazole-3-carboxamide;
1-(4-amino-6-methyl-1,3,5-triazin-2-yl)-N-cyclopentyl-1H-benzimidazol-2-amine
1-(4-amino-6-methyl-1,3,5-triazin-2-yl)-N-3-pyridazinyl-1H-benzimidazol-2-amine;
1-(4-amino-6-methyl-1,3,5-triazin-2-yl)-N-cyclohexyl-1H-benzimidazol-2-amine;
3-((1-(4-amino-6-methyl-1,3,5-triazin-2-yl)-1H-benzimidazol-2-yl)amino)-2-pyrrolidinone;
1-(4-amino-6-methyl-1,3,5-triazin-2-yl)-N-3-pyrrolidinyl-1H-benzimidazol-2-amine;
1-(4-(dimethylamino)-6-methyl-1,3,5-triazin-2-yl)-N-1H-pyrazol-5-yl-1H-benzimidazol-2-amine;
N-(4-((1-(4-amino-6-methyl-1,3,5-triazin-2-yl)-1H-benzimidazol-2-yl)amino)phenyl)-4-tert-butylbenzamide
N-(4-((1-(4-amino-6-methyl-1,3,5-triazin-2-yl)-1H-benzimidazol-2-yl)amino)phenyl)-cyclohexanecarboxamide;
N-(4-((1-(4-amino-6-methyl-1,3,5-triazin-2-yl)-1H-benzimidazol-2-yl)amino)phenyl)-3-(trifluoromethyl)benzamide;
N-(4-((1-(4-amino-6-methyl-1,3,5-triazin-2-yl)-1H-benzimidazol-2-yl)amino)phenyl)-4-(trifluoromethoxy)benzamide;
ethyl 5-((1-(4-amino-6-methyl-1,3,5-triazin-2-yl)-1H-benzimidazol-2-yl)amino)-1H-pyrazole-3-carboxylate;
N-(4-((1-(4-amino-6-methyl-1,3,5-triazin-2-yl)-1H-benzimidazol-2-yl)amino)phenyl)-4-fluorobenzenesulfonamide;
1-(4-amino-6-methyl-1,3,5-triazin-2-yl)-N-(1-(4-morpholinylcarbonyl)-1H-pyrazol-4-yl)-1H-benzimidazol-2-amine;
N-(1-(4-amino-6-methyl-1,3,5-triazin-2-yl)-1H-benzimidazol-2-yl)-1,3-benzoxazol-6-amine; and
4-((1-(4-amino-6-methyl-1,3,5-triazin-2-yl)-1H-benzimidazol-2-yl)amino)-N-4-pyridinyl-1H-pyrazole-1-carboxamide; or
a pharmaceutically acceptable salt thereof.

18. A pharmaceutical composition comprising a compound of claim 1 and a pharmaceutically acceptable excipient.

19. (canceled)

20. (canceled)

21. A method of treating cancer comprising administration of a therapeutically effective amount of a compound of claim 1; or a pharmaceutically acceptable salt thereof.
